(19) United States
(12) Patent Application Publication
(10) Pub. No.: US 2006/0247193 A1
(43) Pub. Date: Nov. 2, 2006

(54) Regulation of gene expression by dna interference

(75) Inventors: Kazunari Taira, Ibaraki (JP); Hiroaki Kawasaki, Tokyo (JP)

Correspondence Address: FOLEY AND LARDNER LLP;SUITE 500, 3000 K STREET NW, WASHINGTON, DC, 20007, US

(73) Assignee: NATIONAL INSTITUTE OF ADVANCED INDUSTRIAL SCIENCE AND TECHNOLOGY

(21) Appl. No.: 10/544,761

(22) PCT Filed: Feb. 10, 2004
(86) PCT No.: PCT/JP04/01433

§ 371 Date: Apr. 4, 2006

Related U.S. Application Data
(60) Provisional application No. 60/445,829, filed on Feb. 10, 2003.

(51) Int. Cl. A61K 48/00; C12N 15/09

(52) U.S. Cl. 514/44; 435/455

(57) ABSTRACT

The present invention provides products and methods for modulating expression of a target gene in a cell. One such method includes introducing into the cell a polynucleotide that forms a duplex region with an mRNA transcribed from said target gene, where the duplex region comprises a mammalian miRNA target region. Another such method includes introducing into the cell an siRNA that forms a duplex region with an miRNA, or precursor thereof, where an mRNA transcribed from the target gene comprises a miRNA target region. In certain preferred embodiments, the methods further include measuring expression of the target gene. The methods are particularly useful for modulating ontogenesis, function, differentiation and/or viability of a mammalian cell. As such, the invention also provides methods for controlling ontogenesis of mammal, function of mammalian cell, differentiation of mammalian cell or viability of mammalian cell in the post-transcriptional phase by introducing into the cell a miRNA or a siRNA silencing precursor to the miRNA. The invention additionally provides polynucleotides, including vectors, useful in the method of the instant invention. The provided polynucleotides include a plasmid vector comprising a promoter and a polynucleotide sequence expressing miRNA or precursor to the miRNA. Also included is a plasmid vector comprising a promoter and a nucleotide sequence expressing siRNA silencing precursor to miRNA. In certain preferred embodiments, the mRNA is capable of forming a duplex region with an mRNA transcribed from a mammalian target gene.

### RELATED APPLICATIONS

This application claims the benefit of U.S. Provisional Application No. 60/445,829, filed Feb. 10, 2003, the entirety of which is herein incorporated by reference.

### FIELD OF THE INVENTION

The invention relates to processes for modulating gene expression in mammalian cells as well as to products and compositions useful in such methods. The methods and compositions are useful, by way of example, for controlling ontogenesis, function, differentiation and/or viability of a mammalian cell.

### BACKGROUND OF THE INVENTION

Noncoding RNAs including rRNA, snRNA, snoRNA and tRNA have roles in a great variety of processes such as chromosome maintenance, gene imprinting, transcriptional regulation, pre-mRNA splicing and the control of mRNA translation¹. One class of the noncoding RNAs called microRNAs (mRNAs) is small RNAs that are known to regulate mRNA at a post-transcriptional level²⁻¹⁸. To date, a large number of miRNAs has been discovered in animals and plants^{2,3,6-16}. Among them, lin-4 and let-7 are identified from the genetic analysis of developmental timing in Caenorhabditis elegans, and are well characterized²⁻⁵. Both lin-4 and let-7 act as repressors of their respective target genes, such as lin-14, lin-28, and lin41. Repression by these mRNAs requires the presence of partially complementary sequences in the 3′-untranslated regions (3′-UTRs) of the target mRNAs. Although lin-14 and lin-28 are translationally repressed by lin-4, these mRNAs were detected in association with polyribosomes^{19,20}. Thus, lin-4 regulates expression of the target genes after translational initiation.

In general, mRNAs are first transcribed as a long RNA and then processed to a pre-mRNA of approximately ˜70 nts²¹. This pre-mRNA is transported to the cytoplasm and processed by RNase III Dicer to produce the mature mRNA²¹⁻²⁴. The mature mRNA is incorporated into ribonucleoprotein complexes (miRNPs) including eIF2C2, which functions in RNA interference (RNAi)-mediated gene silencing^{9,16,25}. This mRNA-miRNPs complex represses mRNA translation by partially base-pairing to the 3′-UTR of target mRNAs^{2-5,26,27}. However, Arabidopsis thagana miR-171 and miR-165/166 are perfectly complementary to the coding region of the Scarecrow-like (SCL) family of the putative transcription factor, PHAVOLUTA (PHV) and PHABULOSA (PHB) mRNA, respectively^{17,18}. These mRNAs can induce cleavage of the mRNAs similar to siRNA-mediated mRNA degradation. Thus, mRNAs have functions including repression of the mRNA translation and cleavage of mRNAs. In general, mRNAs including lin-4 and let-7 control the mRNA translation by partially base-pairing to the 3′-UIR region of target mRNA²⁻⁵. In Arabidopsis thaliana, miR-171 and miR-165/166 are perfectly complementary to coding region of Scarecrow-like (SCL) family mRNA, PHAVOLUTA (PHV) and PHABULOSA (PHB) mRNA, respectively^{17,18}. These mRNAs cleave their target mRNAs, resulting in siRNA-like gene silencing. It has been proposed that there is only a single pathway shared by both mRNAs and siRNAs and that this single pathway mediates both translational control and mRNA cleavage⁴³.

In C. elegans, let-7 and lin-4 are expressed sequentially during development^{2-5,19}. Thus, since mRNAs suppress the expression of the lin-41 and lin-14/28 genes that are necessary for normal development of C. elegans, it is likely that these mRNAs play important roles in development²⁻⁵. In plants, several genes that are targets of mRNAs, including genes in the SCL family, have been identified and their functions have been characterized^{17,18}. SCL family, a target of miR-171, controls a wide range of developmental processes, including radial patterning in roots and hormone signaling. In addition, miR-165/166 can regulate the expression of PHV and PHB genes that encode homeodomain-leucine zipper transcription factors implicated in the perception of radial position in the shoot tissues that give rise to leaves. Moreover, bantam microRNA simultaneously stimulates cell proliferation and prevents apoptosis during Drosophila development⁴⁴. Thus, a number of mRNAs have been identified as playing important roles in the development of animals and plants.

Although more than two hundred mRNAs have been found in mammals, the target mRNAs of these known mRNAs remain to be identified. In view of the well established need in the art for additional means to regulate gene expression in mammalian systems, identifying the mRNA target sequences for those known mammalian miRNAs would have great implications for controlling ontogenesis, function, differentiation and/or viability of a mammalian cell.

### SUMMARY OF THE INVENTION

The present invention provides products and methods for modulating expression of a target gene in a cell. One such method comprises introducing into the cell a polynucleotide that forms a duplex region with an mRNA transcribed from said target gene, wherein the duplex region comprises a mammalian mRNA target region. Another such method comprises introducing into the cell an siRNA that forms a duplex region with an mRNA, or precursor thereof wherein an miRNA transcribed from the target gene comprises a mRNA target region. In certain preferred embodiments, the methods further comprise measuring expression of the target gene. The methods are particularly useful for modulating ontogenesis, function, differentiation and/or viability of a mammalian cell. As such, the invention also provides methods for controlling ontogenesis of mammal, function of mammalian cell, differentiation of mammalian cell or viability of mammalian cell in the post-transcriptional phase by introducing into the cell a mRNA or a siRNA silencing precursor to the mRNA. The invention additionally provides polynucleotides, including mRNAs, siRNAs, and vectors, useful in the method of the instant invention. The provided vectors include a plasmid vector comprising a promoter and a polynucleotide sequence expressing mRNA or a precursor to the mRNA. Also included is a plasmid vector comprising a promoter and a nucleotide sequence expressing siRNA silencing precursor to mRNA. In certain preferred embodiments, the mRNA is capable of forming a duplex region with an mRNA transcribed from a mammalian target gene.

### BRIEF DESCRIPTION OF THE DRAWINGS

FIGS. 1a-1e. Hes1 (NM-005524) is a target of miR-23. a, The prediction of secondary structures between miR-23 and its target RNAs. A region sharing high homology to human and mouse miR-23 is located in the coding region, near the termination codon (box), of human Hairy HES1 (NML005524), mouse Hes1, and human Homolog HES1 (Y07572) mRNAs (top). b, Human Hairy HES1 (NM_{—}005524) mRNA has three target regions (motifs I, II and III) of miR-23 (bottom). Motif m has a K box sequence (black box) that is known, at least in the case of Drosophila, to be involved in post-transcriptional negative regulation. c, The level of Hes1 in NT2 cells in the presence or absence of RA (5 μM, for 3 weeks). Values are means with S.D. of results from three replicates in each case. d, The relative level of Hes1 mRNA in NT2 cells in the presence or absence of RA (5 μM, for 3 weeks). The relative level of Hes1 mRNA was determined by Northern blotting analysis. N; nuclear fraction, C; cytoplasmic fraction. e, The level of miR-23 in NT2 cells in the presence or absence of RA (5 μM, for 3 weeks) was determined by Northern blotting analysis.

FIGS. 2a-2h. Effects of synthetic miR-23 and siRNA-miR-23 targeted to a loop region of the precursor to miR-23 on expression of the gene for Hes1. a, Sequences of synthetic miR-23, double stranded miR-23 and mutant miR-23. Asterisks indicate nucleotides mutated relative to those in the sequence of miR-23. b, The level of HES1 in undifferentiated NT2 cells that had been treated with synthetic miR-23 (100 nM) or with synthetic mutant miR-23 (100 nM) in the absence of RA. Values are means with S.D. of results from three replicates in each case. c, The level of HES1 in undifferentiated NT2 cells that had been treated with synthetic single stranded miR-23 (100 nM) or with synthetic double stranded miR-23 (100 nM) in the absence of RA. d, The level of Hes1 mRNA in undifferentiated NT2 cells that had been treated with synthetic miR-23 or synthetic mutant miR-23 in the absence of RA. N; nuclear fraction, C; cytoplasmic fraction. e, Sequences of synthetic siRNA-miR-23 and synthetic mutant siRNA-miR-23. & The level of precursor and mature miR-23, as detected by Northern blotting analysis in NT2 cells in the presence of RA (5 μM, for 3 weeks). Actin mRNA was used as an endogenous control. g, The level of HES1 in NT2 cells in the presence of RA (5 μM). Values are means with S.D. of results from three replicates in each case. h, The level of Hes1 mRNA in differentiated NT2 cells in the presence of RA (5 μM). N; nuclear fraction, C; cytoplasmic fraction.

FIGS. 3a-3h. Target specificity of miR-23, as determined with plasmids that encoding a gene for luciferase fused to the sequences of three target motifs of miR-23 in Hairy HES1 mRNA and Homolog HES1 mRNA. a, Sequences of genes for Luc-TM23, Luc-mutant TM23 and Luc-mutant motif. The target site of miR-23 or mutant miR-23 is in a black box. Asterisks indicate nucleotides mutated relative to those in the target site of miR-23. b, The activity of luciferase, due to the reporter genes, in NT2 cells in the presence or absence of RA (5 μM). Values are means with S.D. of results from three replicates in each case. c, The activity of luciferase, due to the reporter genes, in undifferentiated NT2 cells in the presence or absence of synthetic miR-23 or mutant miR-23. d, The activitiy of luciferase, due to the reporter genes, in differentiated NT2 cells in the presence or absence of siRNA-miR-23. e, Sequences of genes for Luc-TS23 and mutant Luc-TS23 (Luc-mTS23). The target site of miR-23 or mutant miR-23 is in a blue box. Asterisks indicate nucleotides mutated relative to those in the target site of miR-23. f, The activitiy of luciferase, due to the reporter genes, in NT2 cells in the presence or absence of RA (5 μM). Values are means with S.D. of results from three replicates in each case. g, The activitiy of luciferase, due to the reporter genes, in undifferentiated NT2 cells in the presence or absence of synthetic miR-23 or mutant miR-23. h, The activitiy of luciferase, due to the reporter genes, in differentiated NT2 cells in the presence or absence of siRNA-miR-23.

FIGS. 4a-4c. The role of miR-23 during the RA-induced neuronal differentiation of NT2 cells a, Effects of siRNA-miR-23 on RA-induced neuronal differentiation. Left panel, wild-type NT2 cells after treatment with RA (5 μM, for 3 weeks); middle panel, NT2 cells after treatment with siRNA-miR-23 and RA; right panel, NT2 cells after treatment with siRNA-miR-23, synthetic miR-23 and RA. Nuclei of each NT2 cell were stained with 4-diamidino-2-phenylindole (DAPI). b, The level of MAP2 after RA-induced (5 μM RA) neuronal differentiation. c, The level of SSEA-3 after RA-induced (5 μM RA) neuronal differentiation.

FIG. 5. The effect of various mRNAs on expressions these target mRNAs. The levels of target proteins were analyzed by western blotting and calculated using NIH image program.

Table 1. Identification of target genes for various mRNAs.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, the term “siRNA” refers to a double stranded RNA molecule which binds to a target polyribonucleotide. In a preferred embodiment, binding of the siRNA to the target molecule inhibits the function of the target polyribonucleotide.

As used herein, the term “organism” refers to any living entity comprised of at least one cell. A living organism can be as simple as, for example, a single eukaryotic cell or complex multi-cellular animal, such as a mammal.

As used herein, the term “mammal” refers to members of the class Mammalia, including the primates. Particularly preferred members of the class Mammalia include human, cattle, goat, pig, sheep, rodent, hamster, mouse and rat.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operatively associated with a different gene than the one it is operatively associated with in nature.

As used herein, two polynucleotide sequences are said to be “substantially homologous” or to share “substantial homology” when they share about 70% identity. In a more preferred embodiment, polynucleotides sharing “substantial homology” are those having at least about 80% identity, more preferably at least about 90% identity, and still more preferably, at least about 95% identity. It is additionally preferred that such substantially homologous polynucleotides share a functional similarity. For example, in one particullary preferred embodiment, substantially homologous polynucleotides will hybridize under moderately or highly stringent hybridization conditions. In another preferred embodiment, substantially homologous polynucleotides function to encode polypeptides that share a biologically significant activity characteristic of the polypeptide.

Stringency of hybridization refers to conditions under which polynucleotide duplex is stable. As known to those of skill in the art, the stability of duplex is a function of salt concentration and temperature (See, for example, Sambrook et al., Molecular Cloning: A Laboratory Manual 2d Ed. (Cold Spring Harbor Laboratory, (1989); incorporated herein by reference). Stringency levels used to hybridize a given probe with target-DNA can be readily varied by those of skill in the art. The phrase “low stringency hybridization” refers to conditions equivalent to hybridization in 10% formamide, 5×. Denhart's solution, 6×SSPE, 0.2% SDS at 42 degree C., followed by washing in 1×SSPE, 0.2% SDS, at 50 degrees C. Denhart's solution and SSPE are well known to those of skill in the art as are other suitable hybridization buffers. (See, e.g., Sambrook et al., Molecular Cloning, A Laboratory Manual, Cold Spring Harbor Laboratory Press, 1989)

As used herein, the term “moderately stringent hybridization” refers to conditions that permit target-DNA to bind a complementary nucleic acid that has about 70% identity, preferably about 75% identity, more preferably about 86% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42 degrees C., followed by washing in 0.2×SSPE, 0.2% SDS, at 65 degrees C. Additional examples of typical “moderately stringent conditions” include 0.015 M sodium chloride, 0.0015 M sodium citrate at 50-65 degrees C. or 0.015 M sodium chloride, 0.0015 M sodium citrate, and 20% formamide at 37-50 degrees C. For the purposes of illustration, a “moderately stringent” condition of 50 degrees C. in 0.015 M sodium ion is expected to allow about a 20% mismatch.

The term “highly stringent hybridization” refers to conditions that permit hybridization of only those nucleic acid sequences that that share a high degree identity. High stringency conditions can be provided, for example, by hybridization in 50% formamide, 5× Denhart's solution, 5×SSPE, 0.2% SDS at 42.degrees C., followed by washing in 0.1×SSPE, and 0.1% SDS at 65 degrees C. Additional examples of “highly stringent conditions” for hybridization and washing include 0.015M sodium chloride, 0.0015M sodium citrate at 65-68 degrees C. or 0.015M sodium chloride, 0.0015M sodium citrate, and 50% formamide at 42.degrees.

The “percent identity” between the two sequences is a function of the number of identical positions shared by the sequences. The determination of percent identity between two sequences can be accomplished using any conventional mathematical algorithm, such as the BLAST algorithm by Karlin and Altschul (S. Karlin and S. F. Altschul, Proc. Natl. Acad. Sci. USA. 1990, 87: 2264-2268; S. Karlin and S. F. Altschul, Proc. Natl. Acad. Sci. USA. 1993, 90:5873-5877). The BLAST algorithm is incorporated into the BLASTN program of Altschul et al. (S. F. Altschul et al., J. Mol. Biol. 1990, 215: 403). When a nucleotide sequence is analyzed according to BLASTN, suitable parameters include, for example, a score=100 and word length=12. To obtain gapped alignments for comparison purposes, Gapped BLAST can be utilized as described in Altschul et al. (1997) Nucleic Acids Res. 25:3389. When utilizing BLAST and Gapped BLAST, the default parameters of the respective programs are preferably used. However, one skilled in the art can readily adjust the parameters to suit a particular purpose. Specific procedures for such analysis are known in the art (See, for example, the BLAST website of the National Center for Biotechnology Information.)

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “vector” is a recombinant nucleic acid construct, such as plasmid, phage genome, virus genome, cosmid, or artificial chromosome to which another DNA segment may be attached. In a specific embodiment, the vector may bring about the replication of the attached segment, e.g., in the case of a cloning vector. A “replicon” is any genetic element (e.g., plasmid, chromosome, virus) that functions as an autonomous unit of DNA replication in vivo, i.e., it is capable of replication under its own control. Other preferred examples of vectors include expression vectors comprising expression control sequences.

“Expression control sequences”, e.g., transcriptional and translational control sequences, are regulatory sequences that flank a coding sequence, such as promoters, enhancers, suppressors, terminators, and the like, that provide for the expression of a coding sequence in a host cell. In eukaryotic cells, polyadenylation signals are control sequences. On mRNA, a ribosome binding site is one example of an expression control sequence.

The term “gene” as used herein refers to a portion of a DNA molecule that includes a polypeptide coding sequence operatively associated with one or more expression control sequences. In one embodiment, a gene can be a genomic or partial genomic sequence, in that it contains one or more introns. In another embodiment, a gene can be a cDNA molecule (i.e., the coding sequence lacking any introns).

The gene herein after referred to as “dbl proto-oncogene” (or alternatively as dbl) is well-known in the art. (For a non-limiting example, see GenBank Accession X12556, herein incorporated by reference.) As used herein, the term “dbl proto-oncogene” (as well as dbl) refers to a gene capable of transcribing an mRNA transcript having substantial homology with an mRNA transcribed from the gene for the dbl proto-oncogene set forth in SEQ ID No:291. In a preferred embodiment an mRNA transcribed from said gene comprises an mRNA target region having substantial homology to an mRNA target region set forth in SEQ ID No: 121.

The gene herein after referred to as “transforming growth factor beta 1” (or alternatively as TGFBI) is well-known in the art. (For a non-limiting example, see GenBank Accession NM_{—}000660, herein incorporated by reference.) As used herein, the term “transforming growth factor beta” (as well as TGFBI) refers to a gene capable of transcribing an mRNA transcript having substantial homology with an mRNA transcribed from the gene for the gene for the transforming growth factor beta set forth in SEQ ID No:292. In a preferred embodiment an mRNA transcribed from said gene comprises an mRNA target region having substantial homology to an mRNA target region set forth in SEQ ID No: 122.

The gene herein after referred to as “transforming growth factor alpha” (or alternatively as TGFA or TGF alpha) is well-known in the art. (For a non-limiting example, see GenBank Accession NM_{—}003236, herein incorporated by reference.) As used herein, the term “transforming growth factor alpha” (as well as TGFA or TGF alpha) refers to a gene capable of transcribing an mRNA transcript having substantial homology with an mRNA transcribed from the gene for the transforming growth factor alpha set forth in SEQ ID No:293. In a preferred embodiment an mRNA transcribed from said gene comprises an mRNA target region having substantial homology to an mRNA target region set forth in SEQ ID No: 123.

The gene herein after referred to as “v-myb myeloblastosis viral oncogene homolog” (or alternatively as V-myb or MYB) is well-known in the art. (For a non-limiting example, see GenBank Accession NM_{—}005375, herein incorporated by reference.) As used herein, the term “v-myb myeloblastosis viral oncogene homolog” (as well as V-myb or MYB) refers to a gene capable of transcribing an mRNA transcript having substantial homology with an mRNA transcribed from the gene for the v-myb myeloblastosis viral oncogene homolog set forth in SEQ ID No:294. In a preferred embodiment an mRNA transcribed from said gene comprises an mRNA target region having substantial homology to an mRNA target region set forth in one or more of SEQ ID Nos: 124 and 185.

The gene herein after referred to as “c-cbl proto-oncogene” (or alternatively as c-cbl) is well-known in the art. (For a non-limiting example, see GenBank Accession X57110, herein incorporated by reference.) As used herein, the term “c-cbl proto-oncogene” (as well as c-cbl) refers to a gene capable of transcribing an mRNA transcript having substantial homology with an mRNA transcribed from the gene for the c-cbl proto-oncogene set forth in SEQ ID No:295. In a preferred embodiment an mRNA transcribed from said gene comprises an mRNA target region having substantial homology to an mRNA target region set forth in SEQ ID No: 125.

The gene herein after referred to as “snoI” (or alternatively as SNO I) is well-known in the art. (For a non-limiting example, see GenBank Accession Z19588, herein incorporated by reference.) As used herein, the term “snorI” (as well as SNO I) refers to a gene capable of transcribing an mRNA transcript having substantial homology with an mRNA transcribed from the gene for the snoI set forth in SEQ ID No:296. In a preferred embodiment an mRNA transcribed from said gene comprises an mRNA target region having substantial homology to an mRNA target region set forth in SEQ ID No: 126.

The gene herein after referred to as “activin beta E subunit” (or alternatively as Activin beta) is well-known in the art. (For a non-limiting example, see GenBank Accession AF412024, herein incorporated by reference.) As used herein, the term “activin beta E subunit” (as well as Activin beta) refers to a gene capable of transcribing an mRNA transcript having substantial homology with an mRNA transcribed from the gene for the activin beta E subunit set forth in SEQ ID No:297. In a preferred embodiment an mRNA transcribed from said gene comprises an mRNA target region having substantial homology to an mRNA target region set forth in SEQ ID No: 127.

The gene herein after referred to as “myogenic factor 5” (or alternatively as Myf-5 or MYF5) is well-known in the art. (For a non-limiting example, see GenBank Accession NM_{—}005593, herein incorporated by reference.) As used herein, the term “myogenic factor 5” (as well as Myf-5 or MYF5) refers to a gene capable of transcribing an mRNA transcript having substantial homology with an mRNA transcribed from the gene for the myogenic factor 5 set forth in SEQ ID No:298. In a preferred embodiment an mRNA transcribed from said gene comprises an mRNA target region having substantial homology to an mRNA target region set forth in one or more of SEQ ID Nos:″128 and 267.

The gene herein after referred to as “fibroblast growth factor 9” (or alternatively as FGF9 or glia-activating factor) is well-known in the art. (For a non-limiting example, see GenBank Accession NM_{—}002010, herein incorporated by reference.) As used herein, the term “fibroblast growth factor 9” (as well as FGF9 and glia-activating factor) refers to a gene capable of transcribing an mRNA transcript having substantial homology with an mRNA transcribed from the gene for the fibroblast growth factor 9 set forth in SEQ ID No:299. In a preferred embodiment an m-RNA transcribed from said gene comprises an mRNA target region having substantial homology to an mRNA target region set forth in SEQ ID No: 129.

The gene herein after referred to as “RON encoding a tyrosine kinase” (or alternatively as RON) is well-known in the art. (For a non-limiting example, see GenBank Accession X70040, herein incorporated by reference.) As used herein, the term “RON encoding a tyrosine kinase” (as well as RON) refers to a gene capable of transcribing an mRNA transcript having substantial homology with an mRNA transcribed from the gene for the RON encoding a tyrosine kinase set forth in SEQ ID No:300. In a preferred embodiment an mRNA transcribed from said gene comprises an mRNA target region having substantial homology to an mRNA target region set forth in SEQ ID No: 130.

The gene herein after referred to as “E3 ubiquitin ligase SMURF1” (or alternatively as SMURF1) is well-known in the art. (For a non-limiting example, see GenBank Accession NM_{—}020429, herein incorporated by reference.) As used herein, the term “E3 ubiquitin ligase SMURF1” (as well as SMURF1) refers to a gene capable of transcribing an mRNA transcript having substantial homology with an mRNA transcribed from the gene for the E3 ubiquitin ligase SMURF1 set forth in SEQ ID No:301. In a preferred embodiment an mRNA transcribed from said gene comprises an mRNA target region having substantial homology to an mRNA target region set forth in SEQ ID No: 131.

The gene herein after referred to as “jagged 2” (or alternatively as JAG2) is well-known in the art. (For a non-limiting example, see GenBank Accession NM_{—}002226, herein incorporated by reference.) As used herein, the term “jagged 2” (as well as JAG2) refers to a gene capable of transcribing an mRNA transcript having substantial homology with an mRNA transcribed from the gene for the jagged 2 set forth in SEQ ID No:302. In a preferred embodiment an mRNA transcribed from said gene comprises an mRNA target region having substantial homology to an mRNA target region set forth in SEQ ID No: 132.

The gene herein after referred to as “jun-B encoding the JUN-B protein” (or alternatively as JunB) is well-known in the art. (For a non-limiting example, see GenBank Accession X51345, herein incorporated by reference.) As used herein, the term “jun-B encoding the JUN-B protein”. (as well as JunB) refers to a gene capable of transcribing an mRNA transcript having substantial homology with an mRNA transcribed from the gene for the jun-B encoding the JUN-B protein set forth in SEQ ID No:303. In a preferred embodiment an mRNA transcribed from said gene comprises an mRNA target region having substantial homology to an mRNA target region set forth in SEQ ID No: 133.

The gene herein after referred to as “methyl-CpG binding domain protein 4” (or alternatively as MBD4) is well-known in the art. (For a non-limiting example, see GenBank Accession NM_{—}003925, herein incorporated by reference.) As used herein, the term “methyl-CpG binding domain protein 4” (as well as MBD4) refers to a gene capable of transcribing an mRNA transcript having substantial homology with an mRNA transcribed from the gene for the methyl-CpG binding domain protein 4 set forth in SEQ ID No:304. In a preferred embodiment an mRNA transcribed from said gene comprises an mRNA target region having substantial homology to an mRNA target region set forth in SEQ ID No: 134.

The gene herein after referred to as “ZIP kinase” (or alternatively as ZIP Kinase) is well-known in the art. (For a non-limiting example, see GenBank Accession AB022341, herein incorporated by reference.) As used herein, the term “ZIP kinase” (as well as ZIP Kinase) refers to a gene capable of transcribing an mRNA transcript having substantial homology with an m-RNA transcribed from the gene for the ZIP kinase set forth in SEQ ID No:305. In a preferred embodiment an mRNA transcribed from said gene comprises an mRNA target region having substantial homology to an miRNA target region set forth in SEQ ID No: 135.

The gene herein after referred to as “endomucin” (or alternatively as Endomucin or EMCN) is well-known in the art. (For a non-limiting example, see GenBank Accession NM_{—}016242, herein incorporated by reference.) As used herein, the term “endomucin” (as well as Endomucin or EMCN) refers to a gene capable of transcribing an mRNA transcript having substantial homology with an mRNA transcribed from the gene for the endomucin set forth in SEQ ID No:306. In a preferred embodiment an mRNA transcribed from said gene comprises an mRNA target region having substantial homology to an mRNA target region set forth in SEQ ID No: 136.

The gene herein after referred to as “ICE-protease activating factor” (or alternatively as IPAF) is well-known in the art. (For a non-limiting example, see GenBank Accession AY035391, herein incorporated by reference.) As used herein, the term “ICE-protease activating factor” (as well as IPAF) refers to a gene capable of transcribing an mRNA transcript having substantial homology with an mRNA transcribed from the gene for the ICE-protease activating factor set forth in SEQ ID No:307. In a preferred embodiment an mRNA transcribed from said gene comprises an mRNA target region having substantial homology to an mRNA target region set forth in SEQ ID No: 137.

The gene herein after referred to as “hairy and enhancer of split 1” (or alternatively as HES1) is well-known in the art. (For a non-limiting example, see GenBank Accession NM_{—}005524, herein incorporated by reference.) As used herein, the term “hairy and enhancer of split 1” (as well as Hes1) refers to a gene capable of transcribing an mRNA transcript having substantial homology with an mRNA transcribed from the gene for the hairy and enhancer of split 1 set forth in SEQ ID No:308. In a preferred embodiment an mRNA transcribed from said gene comprises an mRNA target region having substantial homology to an mRNA target region set forth in one or more of SEQ ID Nos: 5, 6, 7 and 171.

The gene herein after referred to as “transforming growth factor beta 3” (or alternatively as TGF-B3 or TGFB3) is well-known in the art. (For a non-limiting example, see GenBank Accession NM_{—}003239, herein incorporated by reference.) As used herein, the term “transforming growth factor beta 3” (as well as TGF-B3 or TGFB3) refers to a gene capable of transcribing an mRNA transcript having substantial homology with an mRNA transcribed from the gene for the transforming growth factor beta 3 set forth in SEQ ID No:309. In a preferred embodiment an mRNA transcribed from said gene comprises an mRNA target region having substantial homology to an mRNA target region set forth in SEQ ID No: 138.

The gene herein after referred to as “enaptin mRNA” (or alternatively as enaptin) is well-known in the art. (For a non-limiting example, see GenBank Accession AF535142, herein incorporated by reference.) As used herein, the term “enaptin mRNA” (as well as enaptin) refers to a gene capable of transcribing an mRNA transcript having substantial homology with an mRNA transcribed from the gene for the enaptin mRNA set forth in SEQ ID No:310. In a preferred embodiment an mRNA transcribed from said gene comprises an mRNA target region having substantial homology to an mRNA target region set forth in SEQ ID No: 139.

The gene herein after referred to as “AMP deaminase” (or alternatively as AMPD3) is well-known in the art. (For a non-limiting example, see GenBank Accession M84721, herein incorporated by reference.) As used herein, the term “AMP deaminase” (as well as AMPD3) refers to a gene capable of transcribing an mRNA transcript having substantial homology with an mRNA transcribed from the gene for the AMP deaminase set forth in SEQ ID No:311. In a preferred embodiment an mRNA transcribed from said gene comprises an mRNA target region having substantial homology to an mRNA target region set forth in SEQ ID No: 140.

The gene herein after referred to as “interleukin 1 alpha” (or alternatively as IL1A) is well-known in the art. (For a non-limiting example, see GenBank Accession AF536338, herein incorporated by reference.) As used herein, the term “interleukin 1 alpha” (as well as IL1A) refers to a gene capable of transcribing an mRNA transcript having substantial homology with an mRNA transcribed from the gene for the interleukin 1 alpha set forth in SEQ ID No:312. In a preferred embodiment an mRNA transcribed from said gene comprises an mRNA target region having substantial homology to an mRNA target region set forth in SEQ ID No: 141.

The gene herein after referred to as “E2F transcription factor 6” (or alternatively as E2F6) is well-known in the art. (For a non-limiting example, see GenBank Accession NM_{—}001952, herein incorporated by reference.) As used herein, the term “E2F transcription factor 6” (as well as E2F6) refers to a gene capable of transcribing an mRNA transcript having substantial homology with an mRNA transcribed from the gene for the E2F transcription factor 6 set forth in SEQ ID No:313. In a preferred embodiment an mRNA transcribed from said gene comprises an mRNA target region having substantial homology to an mRNA target region set forth in SEQ ID No: 142.

The gene herein after referred to as “laminin alpha” (or alternatively as laminin alpha or LAMA) is well-known in the art. (For a non-limiting example, see GenBank Accession NM_{—}005559, herein incorporated by reference.) As used herein, the term “laminin alpha” (as well as laminin alpha or LAMA) refers to a gene capable of transcribing an mRNA transcript having substantial homology with an mRNA transcribed from the gene for the laminin alpha set forth in SEQ ID No: 314. In a preferred embodiment an mRNA transcribed from said gene comprises an mRNA target region having substantial homology to an mRNA target region set forth in SEQ ID No: 143.

The gene herein after referred to as “polymerase (DNA-directed) alpha” (or alternatively as DNA Pol alpha or POLA2) is well-known in the art. (For a non-limiting example, see GenBank Accession NM_{—}002689, herein incorporated by reference.) As used herein, the term “polymerase (DNA-directed) alpha” (as well as DNA Pol alpha or POLA2) refers to a gene capable of transcribing an mRNA transcript having substantial homology with an mRNA transcribed from the gene for the polymerase (DNA-directed) alpha set forth in SEQ ID No:315. In a preferred embodiment an mRNA transcribed from said gene comprises an mRNA target region having substantial homology to an mRNA target region set forth in SEQ ID No: 144.

The gene herein after referred to as “leukocyte tyrosine kinase” (or alternatively as LTK) is well-known in the art. (For a non-limiting example, see GenBank Accession NM_{—}002344, herein incorporated by reference.) As used herein, the term “leukocyte tyrosine kinase” (as well as LTK) refers to a gene capable of transcribing an mRNA transcript having substantial homology with an mRNA transcribed from the gene for the leukocyte tyrosine kinase set forth in SEQ ID No:316. In a preferred embodiment an mRNA transcribed from said gene comprises an mRNA target region having substantial homology to an mRNA target region set forth in SEQ ID No: 145.

The gene herein after referred to as “homeo box D1” (or alternatively as HOXD1) is well-known in the art. (For a non-limiting example, see GenBank Accession NM_{—}024501, herein incorporated by reference.) As used herein, the term “homeo box D1” (as well as HOXD1) refers to a gene capable of transcribing an mRNA transcript having substantial homology with an mRNA transcribed from the gene for the homeo box D1 set forth in SEQ ID No:317. In a preferred embodiment an mRNA transcribed from said gene comprises an mRNA target region having substantial homology to an mRNA target region set forth in SEQ ID No: 146.

The gene herein after referred to as “laminin gamma” (or alternatively as LAMB2 or laminin gamma) is well-known in the art. (For a non-limiting example, see GenBank Accession NM_{—}002293, herein incorporated by reference.) As used herein, the term “laminin gamma” (as well as LAMB2 or laminin gamma) refers to a gene capable of transcribing an mRNA transcript having substantial homology with an mRNA transcribed from the gene for the laminin gamma (formerly LAMB2) set forth in SEQ ID No:318. In a preferred embodiment an mRNA transcribed from said gene comprises an mRNA target region having substantial homology to an mRNA target region set forth in SEQ ID No: 147.

The gene herein after referred to as “tumor necrosis factor receptor superfamily member 1A” (or alternatively as TNFR1) is well-known in the art. (For a non-limiting example, see GenBank Accession BC010140, herein incorporated by reference.) As used herein, the term “tumor necrosis factor receptor superfamily member 1A” (as well as TNFR1) refers to a gene capable of transcribing an mRNA transcript having substantial homology with an mRNA transcribed from the gene for the tumor necrosis factor receptor superfamily member 1A set forth in SEQ ID No:319. In a preferred embodiment an mRNA transcribed from said gene comprises an mRNA target region having substantial homology to an mRNA target region set forth in one or more of SEQ ID Nos:″148 and 200.

The gene herein after referred to as “villin 2” (or alternatively as Villin2 or VIL2) is well-known in the art. (For a non-limiting example, see GenBank Accession NM_{—}003379, herein incorporated by reference.) As used herein, the term “villin 2” (as well as Villin2 or VIL2) refers to a gene capable of transcribing an mRNA transcript having substantial homology with an mRNA transcribed from the gene for the villin 2 set forth in SEQ ID No:320. In a preferred embodiment an mRNA transcribed from said gene comprises an mRNA target region having substantial homology to an mRNA target region set forth in SEQ ID No:149.

The gene herein after referred to as “frizzled homolog 5” (or alternatively as Frizzled homolog 5 or FZD5) is well-known in the art. (For a non-limiting example, see GenBank Accession NM_{—}003468, herein incorporated by reference.) As used herein, the term “frizzled homolog 6” (as well as Frizzled homolog 5 or FZD5) refers to a gene capable of transcribing an mRNA transcript having substantial homology with an mRNA transcribed from the gene for the frizzled homolog 5 set forth in SEQ ID No:321. In a preferred embodiment an mRNA transcribed from said gene comprises an mRNA target region having substantial homology to an mRNA target region set forth in SEQ ID No: 150.

The gene herein after referred to as “ATP-dependent chromatin remodelling protein” (or alternatively as ACF1) is well-known in the art. (For a non-limiting example, see GenBank Accession AF213467, herein incorporated by reference.) As used herein, the term “ATP-dependent chromatin remodelling protein” (as well as ACF1) refers to a gene capable of transcribing an mRNA transcript having substantial homology with an mRNA transcribed from the gene for the ATP-dependent chromatin remodelling protein set forth in SEQ ID No:322. In a preferred embodiment an mRNA transcribed from said gene comprises an mRNA target region having substantial homology to an mRNA target region set forth in SEQ ID No: 151.

The gene herein after referred to as “MSX2 mRNA for transcription factor” (or alternatively as MSX2) is well-known in the art. (For a non-limiting example, see GenBank Accession X69295, herein incorporated by reference.) As used herein, the term “MSX2 mRNA for transcription factor” (as well as MSX2) refers to a gene capable of transcribing an mRNA transcript having substantial homology with an mRNA transcribed from the gene for the MSX2 mRNA for transcription factor set forth in SEQ ID No:323. In a preferred embodiment an mRNA transcribed from said gene comprises an mRNA target region having substantial homology to an mRNA target region set forth in SEQ ID No:152.

The gene herein after referred to as “adipose differentiation-related protein” (or alternatively as ADFP) is well-known in the art. (For a non-limiting example, see GenBank Accession NM_{—}001122, herein incorporated by reference.) As used herein, the term “adipose differentiation-related protein” (as well as ADFP) refers to a gene capable of transcribing an mRNA transcript having substantial homology with an mRNA transcribed from the gene for the adipose differentiation-related protein set forth in SEQ ID No:324. In a preferred embodiment an mRNA transcribed from said gene comprises an mRNA target region having substantial homology to an mRNA target region set forth in SEQ ID No: 153.

The gene herein after referred to as “myogenic factor 4” (or alternatively as myogenin or Myf-4 or MYOG) is well-known in the art. For a non-limiting example, see GenBank Accession NM_{—}002479, herein incorporated by reference.) As used herein, the term “myogenic factor 4” (as well as myogenin or Myf-4 or MYOG) refers to a gene capable of transcribing an mRNA transcript having substantial homology with an mRNA transcribed from the gene for the myogenin (myogenic factor 4) set forth in SEQ ID No:325. In a preferred embodiment an mRNA transcribed from said gene comprises an mRNA target region having substantial homology to an mRNA target region set forth in SEQ ID No:154.

The gene herein after referred to as “SRY (Sex determining Region Y)-box 5” (or alternatively as Sox-6 or SOX5) is well-known in the art. (For a non-limiting example, see GenBank Accession NM_{—}006940, herein incorporated by reference.) As used herein, the term “SRY (Sex determining Region Y)-box 5” (as well as Sox-5 or SOX5) refers to a gene capable of transcribing an mRNA transcript having substantial homology with an mRNA transcribed from the gene for the SRY (Sex determining Region Y)-box 5 set forth in SEQ ID No:326. In a preferred embodiment an mRNA transcribed from said gene comprises an mRNA target region having substantial homology to an mRNA target region set forth in SEQ ID No: 155.

The gene herein after referred to as “Notch homolog 1” (or alternatively as Notch1) is well-known in the art. For a non-limiting example, see GenBank Accession NM_{—}017617, herein incorporated by reference.) As used herein, the term “Notch homolog 1” (as well as Notch1) refers to a gene capable of transcribing an mRNA transcript having substantial homology with an mRNA transcribed from the gene for the Notch homolog 1 set forth in SEQ ID No:327. In a preferred embodiment an mRNA transcribed from said gene comprises an mRNA target region having substantial homology to an mRNA target region set forth in SEQ ID No: 156.

The gene herein after referred to as “Human tyrosine kinase-type receptor” (or alternatively as ErbB2 or HER2) is well-known in the art. (For a non-limiting example, see GenBank Accession M11730, herein incorporated by reference.) As used herein, the term “Human tyrosine kinase-type receptor” (as well as ErbB2 or HER2) refers to a gene capable of transcribing an mRNA transcript having substantial homology with an mRNA transcribed from the gene for the Human tyrosine kinase-type receptor set forth in SEQ ID No:328. In a preferred embodiment an mRNA transcribed from said gene comprises an mRNA target region having substantial homology to an mRNA target region set forth in SEQ ID No: 157.

The gene herein after referred to as “polymerase (DNA directed) theta” (or alternatively as DNA Pol theta or POLQ) is well-known in the art. (For a non-limiting example, see GenBank Accession NM_{—}006596, herein incorporated by reference.) As used herein, the term “polymerase (DNA directed) theta” (as well as DNA Pol theta or POLQ) refers to a gene capable of transcribing an mRNA transcript having substantial homology with an mRNA transcribed from the gene for the polymerase (DNA directed) theta set forth in SEQ ID No:329. In a preferred embodiment an mRNA transcribed from said gene comprises an mRNA target region having substantial homology to an mRNA target region set forth in SEQ ID No:158.

The gene herein after referred to as “cAMP responsive element binding protein 3” (or alternatively as CREB3) is well-known in the art. (For a non-limiting example, see GenBank Accession NM_{—}006368, herein incorporated by reference.) As used herein, the term “cAMP responsive element binding protein 3” (as well as CREB3) refers to a gene capable of transcribing an mRNA transcript having substantial homology with an mRNA transcribed from the gene for the cAMP responsive element binding protein 3 set forth in SEQ ID No:330. In a preferred embodiment an mRNA transcribed from said gene comprises an mRNA target region having substantial homology to an mRNA target region set forth in one or more of SEQ ID Nos:″159 and 163.

The gene herein after referred to as “timeless homolog” (or alternatively as Timeless) is well-known in the art. (For a non-limiting example, see GenBank Accession BC050557, herein incorporated by reference.) As used herein, the term “timeless homolog” (as well as Timeless) refers to a gene capable of transcribing an mRNA transcript having substantial homology with an mRNA transcribed from the gene for the timeless homolog set forth in SEQ ID No:331. In a preferred embodiment an mRNA transcribed from said gene comprises an mRNA target region having substantial homology to an mRNA target region set forth in SEQ ID No:160.

The gene herein after referred to as “RAD52 homolog” (or alternatively as RAD52) is well-known in the art. (For a non-limiting example, see GenBank Accession NM_{—}002879, herein incorporated by reference.) As used herein, the term “RAD52 homolog” (as well as RAD52) refers to a gene capable of transcribing an mRNA transcript having substantial homology with an mRNA transcribed from the gene for the RAD52 homolog set forth in SEQ ID No:332. In a preferred embodiment an mRNA transcribed from said gene comprises an mRNA target region having substantial homology to an mRNA target region set forth in SEQ ID No: 161.

The gene herein after referred to as “toll-like receptor 4′ (or alternatively as TLR4) is well-known in the art. (For a non-limiting example, see GenBank Accession NM_{—}138554, herein incorporated by reference.) As used herein, the term “toll-like receptor 4” (as well as TLR4) refers to a gene capable of transcribing an mRNA transcript having substantial homology with an mRNA transcribed from the gene for the toll-like receptor 4 set forth in SEQ ID No:333. In a preferred embodiment an mRNA transcribed from said gene comprises an mRNA target region having substantial homology to an mRNA target region set forth in SEQ ID No: 162.

The gene herein after referred to as “SRY (Sex determining Region Y)-box 9” (or alternatively as SOX9) is well-known in the art. (For a non-limiting example, see GenBank Accession NM_{—}000346, herein incorporated by reference.) As used herein, the term “SRY (Sex determining Region Y)-box 9” (as well as SOX9) refers to a gene capable of transcribing an mRNA transcript having substantial homology with an mRNA transcribed from the gene for the SRY (Sex determining Region Y)-box 9 set forth in SEQ ID No:334. In a preferred embodiment an mRNA transcribed from said gene comprises an mRNA target region having substantial homology to an mRNA target region set forth in SEQ ID No: 164.

The gene herein after referred to as “homeo box A5” (or alternatively as HOXA5) is well-known in the art. (For a non-limiting example, see GenBank Accession NM_{—}019102, herein incorporated by reference.) As used herein, the term “homeo box A5” (as well as HOXA5) refers to a gene capable of transcribing an mRNA transcript having substantial homology with an mRNA transcribed from the gene for the homeo box A5 set forth in SEQ ID No:335. In a preferred embodiment an mRNA transcribed from said gene comprises an mRNA target region having substantial homology to an mRNA target region set forth in SEQ ID No: 165.

The gene herein after referred to as “cell division cycle 42 GTP binding protein” (or alternatively as CDC42) is well-known in the art. (For a non-limiting example, see GenBank Accession BC018266, herein incorporated by reference.) As used herein, the term “cell division cycle 42 GTP binding protein” (as well as CDC42) refers to a gene capable of transcribing an mRNA transcript having substantial homology with an mRNA transcribed from the gene for the cell division cycle 42 GTP binding protein set forth in SEQ ID No:336. In a preferred embodiment an mRNA transcribed from said gene comprises an mRNA target region having substantial homology to an mRNA target region set forth in SEQ ID No: 166.

The gene herein after referred to as “desmuslin” (or alternatively as DMN) is well-known in the art. (For a non-limiting example, see GenBank Accession NM_{—}145728, herein incorporated by reference.) As used herein, the term “desmuslin” (as well as DMN) refers to a gene capable of transcribing an mRNA transcript having substantial homology with an mRNA transcribed from the gene for the desmuslin set forth in SEQ ID No:337. In a preferred embodiment an mRNA transcribed from said gene comprises an mRNA target region having substantial homology to an mRNA target region set forth in SEQ ID No: 167.

The gene herein after referred to as “TFIIIC Box B-binding subunit” (or alternatively as TFIIIC Box B-binding subunit) is well-known in the art. (For a non-limiting example, see GenBank Accession U02619, herein incorporated by reference.) As used herein, the term “TFIIIC Box B-binding subunit” (as well as TFIIIC Box B-binding subunit) refers to a gene capable of transcribing an mRNA transcript having substantial homology with an mRNA transcribed from the gene for the TFIIIC Box B-binding subunit set forth in SEQ ID No:338. In a preferred embodiment an mRNA transcribed from said gene comprises an mRNA target region having substantial homology to an mRNA target region set forth in one or more of SEQ ID Nos:″168 and 169.

The gene herein after referred to as “profilin 2” (or alternatively as PFN2) is well-known in the art. (For a non-limiting example, see GenBank Accession NM_{—}053024, herein incorporated by reference.) As used herein, the term “profilin 2” (as well as PFN2) refers to a gene capable of transcribing an mRNA transcript having substantial homology with an mRNA transcribed from the gene for the profilin 2 set forth in SEQ ID No:339. In a preferred embodiment an mRNA transcribed from said gene comprises an mRNA target region having substantial homology to an mRNA target region set forth in SEQ ID No: 169.

The gene herein after referred to as “c-fms proto-oncogene” (or alternatively as c-fms) is well-known in the art. (For a non-limiting example, see GenBank Accession X03663, herein incorporated by reference.) As used herein, the term “c-fms proto-oncogene” (as well as c-fms) refers to a gene capable of transcribing an m-RNA transcript having substantial homology with an mRNA transcribed from the gene for the c-fms proto-oncogene set forth in SEQ ID No:340. In a preferred embodiment an mRNA transcribed from said gene comprises an mRNA target region having substantial homology to an mRNA target region set forth in SEQ ID No: 170.

The gene herein after referred to as “delta-like 1” (or alternatively as Deltalor DLL1) is well-known in the art. (For a non-limiting example, see GenBank Accession NM_{—}005618, herein incorporated by reference.) As used herein, the term “delta-like 1” (as well as Deltalor DLL1) refers to a gene capable of transcribing an mRNA transcript having substantial homology with an mRNA transcribed from the gene for the delta-like 1 set forth in SEQ ID No:341. In a preferred embodiment an mRNA transcribed from said gene comprises an mRNA target region having substantial homology to an mRNA target region set forth in SEQ ID No:172.

The gene herein after referred to as “fatty-acid-Coenzyme A ligase long-chain 5” (or alternatively as FACL5) is well-known in the art. (For a non-limiting example, see GenBank Accession NM_{—}016234, herein incorporated by reference.) As used herein, the term fatty-acid-Coenzyme A ligase long-chain 5” (as well as FACL5) refers to a gene capable of transcribing an mRNA transcript having substantial homology with an mRNA transcribed from the gene for the fatty-acid-Coenzyme A ligase long-chain 5 set forth in SEQ ID No:342. In a preferred embodiment an mRNA transcribed from said gene comprises an mRNA target region having substantial homology to an mRNA target region set forth in SEQ ID No:173.

The gene herein after referred to as “discs large homolog-associated protein 2” (or alternatively as DLGAP2) is well-known in the art. (For a non-limiting example, see GenBank Accession NM_{—}004745, herein incorporated by reference.) As used herein, the term “discs large homolog-associated protein 2” (as well as DLGAP2) refers to a gene capable of transcribing an mRNA transcript having substantial homology with an mRNA transcribed from the gene for the discs large homolog-associated protein 2 set forth in SEQ ID No:343. In a preferred embodiment an mRNA transcribed from said gene comprises an mRNA target region having substantial homology to an mRNA target region set forth in SEQ ID No: 174.

The gene herein after referred to as “TFIIH gene for transcription factor II H” (or alternatively as TFIIH) is well-known in the art. (For a non-limiting example, see GenBank Accession AB088103, herein incorporated by reference.) As used herein, the term “TFIIH gene for transcription factor II H” (as well as TFIIH) refers to a gene capable of transcribing an mRNA transcript having substantial homology with an mRNA transcribed from the gene for the TFIIH gene for transcription factor II H set forth in SEQ ID No:344. In a preferred embodiment an mRNA transcribed from said gene comprises an mRNA target region having substantial homology to an mRNA target region set forth in SEQ ID No: 176.

The gene herein after referred to as “RNA polymerase III subunit RPC” (or alternatively as RPC2) is well-known in the art. (For a non-limiting example, see GenBank Accession NM_{—}018082, herein incorporated by reference.) As used herein, the term “RNA polymerase III subunit RPC” (as well as RPC2) refers to a gene capable of transcribing an mRNA transcript having substantial homology with an mRNA transcribed from the gene for the RNA polymerase III subunit RPC set forth in SEQ ID No:345. In a preferred embodiment an mRNA transcribed from said gene comprises an mRNA target region having substantial homology to an mRNA target region set forth in SEQ ID No: 177.

The gene herein after referred to as “RecQ protein-like 5” (or alternatively as RecQ5) is well-known in the art. (For a non-limiting example, see GenBank Accession NM_{—}004259, herein incorporated by reference.) As used herein, the term “RecQ protein-like 5” (as well as RecQ5) refers to a gene capable of transcribing an mRNA transcript having substantial homology with an mRNA transcribed from the gene for the RecQ protein-like 5 set forth in SEQ ID No:346. In a preferred embodiment an mRNA transcribed from said gene comprises an mRNA target region having substantial homology to an mRNA target region set forth in SEQ ID No: 178.

The gene herein after referred to as “METH2 protein” (or alternatively as METH2) is well-known in the art. (For a non-limiting example, see GenBank Accession AF060153, herein incorporated by reference.) As used herein, the term “METHE2 protein” (as well as METH2) refers to a gene capable of transcribing an mRNA transcript having substantial homology with an mRNA transcribed from the gene for the METH2 protein set forth in SEQ ID No:347. In a preferred embodiment an mRNA transcribed from said gene comprises an mRNA target region having substantial homology to an mRNA target region set forth in SEQ ID No: 179.

The gene herein after referred to as “MOST2 protein” (or alternatively as MOST2) is well-known in the art. (For a non-limiting example, see GenBank Accession NM_{—}020250, herein incorporated by reference.) As used herein, the term “MOST2 protein” (as well as MOST2) refers to a gene capable of transcribing an mRNA transcript having substantial homology with an mRNA transcribed from the gene for the MOST2 protein set forth in SEQ ID No:348. In a preferred embodiment an mRNA transcribed from said gene comprises an mRNA target region having substantial homology to an mRNA target region set forth in SEQ ID No: 180.

The gene herein after referred to as “SRY (Sex determining Region Y)-box 7” (or alternatively as SOX7) is well-known in the art. (For a non-limiting example, see GenBank Accession NM_{—}031439, herein incorporated by reference.) As used herein, the term “SRY (Sex determining Region Y)-box 7” (as well as SOX7) refers to a gene capable of transcribing an mRNA transcript having substantial homology with an mRNA transcribed from the gene for the SRY (Sex determining Region Y)-box 7 set forth in SEQ ID No:349. In a preferred embodiment an mRNA transcribed from said gene comprises an mRNA target region having substantial homology to an mRNA target region set forth in SEQ ID No: 181.

The gene herein after referred to as “integrin beta 1 subunit” (or alternatively as Integrin B1) is well-known in the art. (For a non-limiting example, see GenBank Accession X07979, herein incorporated by reference.) As used herein, the term “integrin beta 1 subunit” (as well as Integrin B1) refers to a gene capable of transcribing an mRNA transcript having substantial homology with an mRNA transcribed from the gene for the integrin beta 1 subunit set forth in SEQ ID No:350. In a preferred embodiment an mRNA transcribed from said gene comprises an mRNA target region having substantial homology to an mRNA target region set forth in SEQ ID No:182.

The gene herein after referred to as “desmin” (or alternatively as DES) is well-known in the art. (For a non-limiting example, see GenBank Accession NM_{—}001927, herein incorporated by reference.) As used herein, the term “desmin” (as well as DES) refers to a gene capable of transcribing an mRNA transcript having substantial homology with an mRNA transcribed from the gene for the desmin set forth in SEQ ID No:351. In a preferred embodiment an mRNA transcribed from said gene comprises an mRNA target region having substantial homology to an mRNA target region set forth in SEQ ID No:183.

The gene herein after referred to as “protection of telomeres 1” (or alternatively as POT1) is well-known in the art. (For a non-limiting example, see GenBank Accession NM_{—}015450, herein incorporated by reference.) As used herein, the term “protection of telomeres 1” (as well as POT1) refers to a gene capable of transcribing an mRNA transcript having substantial homology with an mRNA transcribed from the gene for the protection of telomeres 1 set forth in SEQ ID No:352. In a preferred embodiment an mRNA transcribed from said gene comprises an mRNA target region having substantial homology to an mRNA target region set forth in one or more of SEQ ID Nos:″184 and 195.

The gene herein after referred to as “H2.0-like homeo box 1” (or alternatively as HDX1) is well-known in the art. (For a non-limiting example, see GenBank Accession NM_{—}021958, herein incorporated by reference.) As used herein, the term “H2.0-like homeo box 1” (as well as HLX1) refers to a gene capable of transcribing an mRNA transcript having substantial homology with an mRNA transcribed from the gene for the H2.0-like homeo box 1 set forth in SEQ ID No:353. In a preferred embodiment an mRNA transcribed from said gene comprises an mRNA target region having substantial homology to an mRNA target region set forth in SEQ ID No: 186.

The gene herein after referred to as “GABA transport protein” (or alternatively as GABA Transport protein) is well-known in the art. (For a non-limiting example, see GenBank Accession U76343, herein incorporated by reference.) As used herein, the term “GABA transport protein” (as well as GABA Transport protein) refers to a gene capable of transcribing an mRNA transcript having substantial homology with an mRNA transcribed from the gene for the GABA transport protein set forth in SEQ ID No:354. In a preferred embodiment an mRNA transcribed from said gene comprises an mRNA target region having substantial homology to an mRNA target region set forth in SEQ ID No: 187.

The gene herein after referred to as “v-myc myelocytomatosis viral related oncogene neuroblastoma derived” (or alternatively as V-myc or MYCN) is well-known in the art. (For a non-limiting example, see GenBank Accession NM_{—}005378, herein incorporated by reference.) As used herein, the term “v-myc myelocytomatosis viral related oncogene neuroblastoma derived” (as well as V-myc or MYCN) refers to a gene capable of transcribing an mRNA transcript having substantial homology with an mRNA transcribed from the gene for the v-myc myelocytomatosis viral related oncogene neuroblastoma derived set forth in SEQ ID No:355. In a preferred embodiment an mRNA transcribed from said gene comprises an mRNA target region having substantial homology to an mRNA target region set forth in SEQ ID No: 188.

The gene herein after referred to as “BAG-family molecular chaperone regulator-5” (or alternatively as BAG5) is well-known in the art. (For a non-limiting example, see GenBank Accession AF095195, herein incorporated by reference.) As used herein, the term “BAG-family molecular chaperone regulator-6” (as well as BAG5) refers to a gene capable of transcribing an mRNA transcript having substantial homology with an mRNA transcribed from the gene for the BAG-family molecular chaperone regulator-5 set forth in SEQ ID No:356. In a preferred embodiment an mRNA transcribed from said gene comprises an mRNA target region having substantial homology to an mRNA target region set forth in SEQ ID No: 189.

The gene herein after referred to as “Human placental bone morphogenic protein” (or alternatively as PLAB) is well-known in the art. (For a non-limiting example, see GenBank Accession U88323, herein incorporated by reference.) As used herein, the term “Human placental bone morphogenic protein” (as well as PLAB) refers to a gene capable of transcribing an mRNA transcript having substantial homology with an mRNA transcribed from the gene for the Human placental bone morphogenic protein set forth in SEQ ID No:357. In a preferred embodiment an mRNA transcribed from said gene comprises an mRNA target region having substantial homology to an mRNA target region set forth in SEQ ID No: 190.

The gene herein after referred to as “retinoblastoma-associated factor 600” (or alternatively as BAF600) is well-known in the art. (For a non-limiting example, see GenBank Accession AF348492, herein incorporated by reference.) As used herein, the term “retinoblastoma-associated factor 600” (as well as BAF600) refers to a gene capable of transcribing an mRNA transcript having substantial homology with an mRNA transcribed from the gene for the retinoblastoma-associated factor 600 set forth in SEQ ID No:358. In a preferred embodiment an mRNA transcribed from said gene comprises an mRNA target region having substantial homology to an mRNA target region set forth in SEQ ID No: 191.

The gene herein after referred to as “ALK-4” (or alternatively as ALK-4) is well-known in the art. (For a non-limiting example, see GenBank Accession Z22536, herein incorporated by reference.) As used herein, the term “ALK-4” (as well as ALK-4) refers to a gene capable of transcribing an mRNA transcript having substantial homology with an mRNA transcribed from the gene for the ALK-4 set forth in SEQ ID No:359. In a preferred embodiment an mRNA transcribed from said gene comprises an mRNA target region having substantial homology to an mRNA target region set forth in SEQ ID No: 192.

The gene herein after referred to as “tolloid-like 2” (or alternatively as TLL2, is well-known in the art. (For a non-limiting example, see GenBank Accession NM_{—}012465, herein incorporated by reference.) As used herein, the term “tolloid-like 2” (as well as TLL2) refers to a gene capable of transcribing an mRNA transcript having substantial homology with an mRNA transcribed from the gene for the tolloid-like 2 set forth in SEQ ID No:360. In a preferred embodiment an mRNA transcribed from said gene comprises an mRNA target region having substantial homology to an mRNA target region set forth in SEQ ID No: 193.

The gene herein after referred to as “RIGB” (or alternatively as RIGB) is well-known in the art. (For a non-limiting example, see GenBank Accession AF525085, herein incorporated by reference.) As used herein, the term “RIGB” (as well as RIGB) refers to a gene capable of transcribing an mRNA transcript having substantial homology with an mRNA transcribed from the gene for the RIGB set forth in SEQ ID No:361. In a preferred embodiment an mRNA transcribed from said gene comprises an mRNA target region having substantial homology to an mRNA target region set forth in SEQ ID No: 194.

The gene herein after referred to as “Human DNA repair helicase” (or alternatively as ERCC3) is well-known in the art. (For a non-limiting example, see GenBank Accession M31899, herein incorporated by reference.) As used herein, the term “Human DNA repair helicase” (as well as ERCC3) refers to a gene capable of transcribing an mRNA transcript having substantial homology with an mRNA transcribed from the gene for the Human DNA repair helicase set forth in SEQ ID No:362. In a preferred embodiment an mRNA transcribed from said gene comprises an mRNA target region having substantial homology to an mRNA target region set forth in SEQ ID No: 196.

The gene herein after referred to as “T-box 22” (or alternatively as TBX22) is well-known in the art. (For a non-limiting example, see GenBank Accession NM_{—}016954, herein incorporated by reference.) As used herein, the term “T-box 22” (as well as TBX22) refers to a gene capable of transcribing an mRNA transcript having substantial homology with an mRNA transcribed from the gene for the T-box 22 set forth in SEQ ID No:363. In a preferred embodiment an mRNA transcribed from said gene comprises an mRNA target region having substantial homology to an mRNA target region set forth in SEQ ID No: 197.

The gene herein after referred to as “BRCA1 associated protein 1” (or alternatively as BAP1) is well-known in the art. (For a non-limiting example, see GenBank Accession AF046581, herein incorporated by reference.) As used herein, the term “BRCA1 associated protein 1” (as well as BAP1) refers to a gene capable of transcribing an mRNA transcript having substantial homology with an mRNA transcribed from the gene for the BRCA1 associated protein 1 set forth in SEQ ID No:364. In a preferred embodiment an mRNA transcribed from said gene comprises an mRNA target region having substantial homology to an mRNA target region set forth in SEQ ID No: 198.

The gene herein after referred to as “Sp3 transcription factor” (or alternatively as SP3(J)) is well-known in the art. (For a non-limiting example, see GenBank Accession NM_{—}003111, herein incorporated by reference.) As used herein, the term “Sp3 transcription factor” (as well as SP3(J)) refers to a gene capable of transcribing an mRNA transcript having substantial homology with an mRNA transcribed from the gene for the Sp3 transcription factor set forth in SEQ ID No:365. In a preferred embodiment an mRNA transcribed from said gene comprises an mRNA target region having substantial homology to an mRNA target region set forth in SEQ ID No: 199.

The gene herein after referred to as “TEF-1 gene” (or alternatively as TEF1(D)) is well-known in the art. (For a non-limiting example, see GenBank Accession X84839, herein incorporated by reference.) As used herein, the term “TEF-1 gene” (as well as TEF1(D)) refers to a gene capable of transcribing an mRNA transcript having substantial homology with an mRNA transcribed from the gene for the TEF-1 gene set forth in SEQ ID No:366. In a preferred embodiment an mRNA transcribed from said gene comprises an mRNA target region having substantial homology to an mRNA target region set forth in SEQ ID No: 201.

The gene herein after referred to as “forkhead box A3” (or alternatively as FOXA3) is well-known in the art. (For a non-limiting example, see GenBank Accession NM_{—}004497, herein incorporated by reference.) As used herein, the term “forkhead box A3” (as well as FOXA3) refers to a gene capable of transcribing an mRNA transcript having substantial homology with an mRNA transcribed from the gene for the forkhead boxA3 set forth in SEQ ID No:367. In a preferred embodiment an mRNA transcribed from said gene comprises an mRNA target region having substantial homology to an mRNA target region set forth in one or more of SEQ ID Nos:″202 and 210.

The gene herein after referred to as “ets family transcription factor ELF2A” (or alternatively as ELF2) is well-known in the art. (For a non-limiting example, see GenBank Accession AF256222, herein incorporated by reference.) As used herein, the term “ets family transcription factor ELF2A” (as well as ELF2) refers to a gene capable of transcribing an mRNA transcript having substantial homology with an mRNA transcribed from the gene for the ets family transcription factor ELF2A set forth in SEQ ID No:368. In a preferred embodiment an mRNA transcribed from said gene comprises an mRNA target region having substantial homology to an mRNA target region set forth in SEQ ID No: 203.

The gene herein after referred to as “microtubule-associated protein 1A” (or alternatively as MAP1A) is well-known in the art. (For a non-limiting example, see GenBank Accession NM_{—}002373, herein incorporated by reference.) As used herein, the term “microtubule-associated protein 1A” (as well as MAP1A) refers to a gene capable of transcribing an mRNA transcript having substantial homology with an mRNA transcribed from the gene for the microtubule-associated protein 1A set forth in SEQ ID No:369. In a preferred embodiment an mRNA transcribed from said gene comprises an mRNA target region having substantial homology to an mRNA target region set forth in SEQ ID No: 204.

The gene herein after referred to as “myosin 5B” (or alternatively as Myosin 5B) is well-known in the art. (For a non-limiting example, see GenBank Accession AY274809, herein incorporated by reference.) As used herein, the term “myosin 5B” (as well as Myosin 5B) refers to a gene capable of transcribing an mRNA transcript having substantial homology with an mRNA transcribed from the gene for the myosin 5B set forth in SEQ ID No:370. In a preferred embodiment an mRNA transcribed from said gene comprises an mRNA target region having substantial homology to an mRNA target region set forth in SEQ ID No: 205.

The gene herein after referred to as “NEDD4-like ubiquitin ligase 1” (or alternatively as NEDL1) is well-known in the art. (For a non-limiting example, see GenBank Accession AB048365, herein incorporated by reference.) As used herein, the term “NEDD4-like ubiquitin ligase 1” (as well as NEDL1) refers to a gene capable of transcribing an mRNA transcript having substantial homology with an mRNA transcribed from the gene for the NEDD4-like ubiquitin ligase 1 set forth in SEQ ID No:371. In a preferred embodiment an mRNA transcribed from said gene comprises an mRNA target region having substantial homology to an mRNA target region set forth in SEQ ID No: 206.

The gene herein after referred to as “Mint1 mRNA” (or alternatively as MINT1) is well-known in the art. (For a non-limiting example, see GenBank Accession AF029106, herein incorporated by reference.) As used herein, the term “Mint1 mRNA” (as well as MINT1) refers to a gene capable of transcribing an mRNA transcript having substantial homology with an mRNA transcribed from the gene for the Mint1 mRNA set forth in SEQ ID No:372. In a preferred embodiment an mRNA transcribed from said gene comprises an mRNA target region having substantial homology to an mRNA target region set forth in SEQ ID No: 207.

The gene herein after referred to as “PARX protein” (or alternatively as PARX) is well-known in the art. (For a non-limiting example, see GenBank Accession AF439781, herein incorporated by reference.) As used herein, the term “PARX protein” (as well as PARX) refers to a gene capable of transcribing an mRNA transcript having substantial homology with an mRNA transcribed from the gene for the PARX protein set forth in SEQ ID No:373. In a preferred embodiment an mRNA transcribed from said gene comprises an mRNA target region having substantial homology to an mRNA target region set forth in SEQ ID No: 208.

The gene herein after referred to as “epidermal growth factor receptor” (or alternatively as ERBB3) is well-known in the art. (or a non-limiting example, see GenBank Accession M29366, herein incorporated by reference.) As used herein, the term “epidermal growth factor receptor” (as well as ERBB3) refers to a gene capable of transcribing an mRNA transcript having substantial homology with an mRNA transcribed from the gene for the epidermal growth factor receptor set forth in SEQ ID No:374. In a preferred embodiment an mRNA transcribed from said gene comprises an mRNA target region having substantial homology to an mRNA target region set forth in SEQ ID No: 209.

The gene herein after referred to as “matrix metalloproteinase 3” (or alternatively as MMP3) is well-known in the art. (For a non-limiting example, see GenBank Accession AF405705, herein incorporated by reference.) As used herein, the term “matrix metalloproteinase 3” (as well as MMP3) refers to a gene capable of transcribing an mRNA transcript having substantial homology with an mRNA transcribed from the gene for the matrix metalloproteinase 3 (stromelysin 1; progelatinase) set forth in SEQ ID No:376. In a preferred embodiment an mRNA transcribed from said gene comprises an mRNA target region having substantial homology to an mRNA target region set forth in SEQ ID No: 211.

The gene herein after referred to as “VE-cadherin” (or alternatively as VE-CADHERIN) is well-known in the art. (For a non-limiting example, see GenBank Accession X79981, herein incorporated by reference.) As used herein, the term “VE-cadherin” (as well as VE-CADHERIN) refers to a gene capable of transcribing an m-RNA transcript having substantial homology with an mRNA transcribed from the gene for the VE-cadherin set forth in SEQ ID No:376. In a preferred embodiment an mRNA transcribed from said gene comprises an mRNA target region having substantial homology to an mRNA target region set forth in SEQ ID No: 212.

The gene herein after referred to as “microtubule-associated protein 2” (or alternatively as MAP2) is well-known in the art. (For a non-limiting example, see GenBank Accession NM_{—}002374, herein incorporated by reference.) As used herein, the term “microtubule-associated protein 2” (as well as MAP2) refers to a gene capable of transcribing an mRNA transcript having substantial homology with an mRNA transcribed from the gene for the microtubule-associated protein 2 set forth in SEQ ID No:377. In a preferred embodiment an mRNA transcribed from said gene comprises an mRNA target region having substantial homology to an mRNA target region set forth in SEQ ID No: 213.

The gene herein after referred to as “TAF7 RNA polymerase II TATA box binding protein (TBP)-associated factor” (or alternatively as TAFII55 or TAF7) is well-known in the art. (For a non-limiting example, see GenBank Accession NM_{—}005642, herein incorporated by reference.) As used herein, the term “TAF7 RNA polymerase II TATA box binding protein (TBP)-associated factor” (as well as TAFII55 or TAF7) refers to a gene capable of transcribing an mRNA transcript having substantial homology with an mRNA transcribed from the gene for the TAF7 RNA polymerase II TATA box binding protein (TBP)-associated factor set forth in SEQ ID No:378. In a preferred embodiment an mRNA transcribed from said gene comprises an mRNA target region having substantial homology to an mRNA target region set forth in SEQ ID No: 214.

The gene herein after referred to as “mitochondrial elongation factor G2” (or alternatively as EFG2) is well-known in the art. (or a non-limiting example, see GenBank Accession NM_{—}032380, herein incorporated by reference.) As used herein, the term “mitochondrial elongation factor G2” (as well as EFG2) refers to a gene capable of transcribing an mRNA transcript having substantial homology with an mRNA transcribed from the gene for the mitochondrial elongation factor G2 set forth in SEQ ID No:379. In a preferred embodiment an mRNA transcribed from said gene comprises an mRNA target region having substantial homology to an mRNA target region set forth in SEQ ID No: 215.

The gene herein after referred to as “eyes absent homolog” (or alternatively as Eab1) is well-known in the art. (For a non-limiting example, see GenBank Accession U71207, herein incorporated by reference.) As used herein, the term “eyes absent homolog” (as well as Eab1) refers to a gene capable of transcribing an mRNA transcript having substantial homology with an mRNA transcribed from the gene for the eyes absent homolog set forth in SEQ ID No:380. In a preferred embodiment an mRNA transcribed from said gene comprises an mRNA target region having substantial homology to an mRNA target region set forth in SEQ ID No: 216.

The gene herein after referred to as “paired box gene 3” (or alternatively as PAX3) is well-known in the art. (For a non-limiting example, see GenBank Accession NM_{—}181457, herein incorporated by reference.) As used herein, the term “paired box gene 3” (as well as PAX3) refers to a gene capable of transcribing an mRNA transcript having substantial homology with an mRNA transcribed from the gene for the paired box gene 3 set forth in SEQ ID No:381. In a preferred embodiment an mRNA transcribed from said gene comprises an mRNA target region having substantial homology to an mRNA target region set forth in SEQ ID No: 217.

The gene herein after referred to as “synaptotagmin I” (or alternatively as Synaptotagmin1(D) 3UTR) is well-known in the art. (For a non-limiting example, see GenBank Accession U19921, herein incorporated by reference.) As used herein, the term “synaptotagmin I” (as well as Synaptotagmin1(D) 3UTR) refers to a gene capable of transcribing an mRNA transcript having substantial homology with an mRNA transcribed from the gene for the synaptotagmin I set forth in SEQ ID No:382. In a preferred embodiment an mRNA transcribed from said gene comprises an mRNA target region having substantial homology to an mRNA target region set forth in SEQ ID No: 218.

The gene herein after referred to as “histone deacetylase 5′ (or alternatively as HDAC5) is well-known in the art. (For a non-limiting example, see GenBank Accession NM_{—}005474, herein incorporated by reference.) As used herein, the term “histone deacetylase 5” (as well as HDAC5) refers to a gene capable of transcribing an mRNA transcript having substantial homology with an mRNA transcribed from the gene for the histone deacetylase 5 set forth in SEQ ID No:383. In a preferred embodiment an mRNA transcribed from said gene comprises an mRNA target region having substantial homology to an mRNA target region set forth in SEQ ID No: 219.

The gene herein after referred to as “homolog of Drosophila headcase” (or alternatively as hHDC) is well-known in the art. (For a non-limiting example, see GenBank Accession AB033492, herein incorporated by reference.) As used herein, the term “homolog of Drosophila headcase” (as well as hHDC) refers to a gene capable of transcribing an mRNA transcript having substantial homology with an mRNA transcribed from the gene for the homolog of Drosophila headcase set forth in SEQ ID No:384. In a preferred embodiment an mRNA transcribed from said gene comprises an mRNA target region having substantial homology to an mRNA target region set forth in SEQ ID No: 220.

The gene herein after referred to as “homeo box B8” (or alternatively as HOXB8) is well-known in the art. (For a non-limiting example, see GenBank Accession NM_{—}024016, herein incorporated by reference.) As used herein, the term “homeo box B8” (as well as HOXB8) refers to a gene capable of transcribing an mRNA transcript having substantial homology with an mRNA transcribed from the gene for the homeo box B8 set forth in SEQ ID No:385. In a preferred embodiment an mRNA transcribed from said gene comprises an mRNA target region having substantial homology to an mRNA target region set forth in SEQ ID No: 221.

The gene herein after referred to as “fyn-related kinase” (or alternatively as FRK) is well-known in the art. (For a non-limiting example, see GenBank Accession NM_{—}002031, herein incorporated by reference.) As used herein, the term “fyn-related kinase” (as well as FRK) refers to a gene capable of transcribing an mRNA transcript having substantial homology with an mRNA transcribed from the gene for the fyn-related kinase set forth in SEQ ID No:386. In a preferred embodiment an mRNA transcribed from said gene comprises an mRNA target region having substantial homology to an mRNA target region set forth in SEQ ID No: 222.

The gene herein after referred to as “TGF-beta/activin signal transducer FAST-1p” (or alternatively as FAST1) is well-known in the art. (For a non-limiting example, see GenBank Accession AF076292, herein incorporated by reference.) As used herein, the term “TGF-beta/activin signal transducer FAST-1p” (as well as FAST1) refers to a gene capable of transcribing an mRNA transcript having substantial homology with an mRNA transcribed from the gene for the TGF-beta/activin signal transducer FAST-1p set forth in SEQ ID No:387. In a preferred embodiment an mRNA transcribed from said gene comprises an mRNA target region having substantial homology to an mRNA target region set forth in SEQ ID No: 223.

The gene herein after referred to as “La autoantigen” (or alternatively as La antigen) is well-known in the art. (For a non-limiting example, see GenBank Accession X97869, herein incorporated by reference.) As used herein, the term “La autoantigen” (as well as La antigen) refers to a gene capable of transcribing an mRNA transcript having substantial homology with an mRNA transcribed from the gene for the La autoantigen set forth in SEQ ID No:388. In a preferred embodiment an mRNA transcribed from said gene comprises an mRNA target region having substantial homology to an miRNA target region set forth in SEQ ID No: 224.

The gene herein after referred to as “mutL homolog 1” (or alternatively as MLH1) is well-known in the art. Nor a non-limiting example, see GenBank Accession NM_{—}000249, herein incorporated by reference.) As used herein, the term “mutL homolog 1” (as well as MLH1) refers to a gene capable of transcribing an mRNA transcript having substantial homology with an mRNA transcribed from the gene for the mutL homolog 1 set forth in SEQ ID No:389. In a preferred embodiment an mRNA transcribed from said gene comprises an mRNA target region having substantial homology to an mRNA target region set forth in SEQ ID No: 225.

The gene herein after referred to as “E74-like factor 3” (or alternatively as ELF3) is well-known in the art. (For a non-limiting example, see GenBank Accession AF517841, herein incorporated by reference.) As used herein, the term “E74-like factor 3” (as well as ELF3) refers to a gene capable of transcribing an mRNA transcript having substantial homology with an mRNA transcribed from the gene for the E74-like factor 3 set forth in SEQ ID No:390. In a preferred embodiment an mRNA transcribed from said gene comprises an mRNA target region having substantial homology to an mRNA target region set forth in SEQ ID No: 226.

The gene herein after referred to as “B-myb gene” (or alternatively as B-Myb) is well-known in the art. (For a non-limiting example, see GenBank Accession X13293, herein incorporated by reference.) As used herein, the term “B-myb gene” (as well as B-Myb) refers to a gene capable of transcribing an mRNA transcript having substantial homology with an mRNA transcribed from the gene for the B-myb gene set forth in SEQ ID No:391. In a preferred embodiment an mRNA transcribed from said gene comprises an mRNA target region having substantial homology to an mRNA target region set forth in one or more of SEQ ID Nos:″227 and 259.

The gene herein after referred to as “a-myb mRNA” (or alternatively as a-myb) is well-known in the art. (For a non-limiting example, see GenBank Accession X66087, herein incorporated by reference.) As used herein, the term “a-myb mRNA” (as well as a-myb) refers to a gene capable of transcribing an mRNA transcript having substantial homology with an mRNA transcribed from the gene for the a-myb mRNA set forth in SEQ ID No:392. In a preferred embodiment an mRNA transcribed from said gene comprises an mRNA target region having substantial homology to an mRNA target region set forth in SEQ ID No: 228.

The gene herein after referred to as “jagged 1” (or alternatively as JAG1) is well-known in the art. (For a non-limiting example, see GenBank Accession NM_{—}000214, herein incorporated by reference.) As used herein, the term “jagged 1” (as well as JAG1) refers to a gene capable of transcribing an mRNA transcript having substantial homology with an mRNA transcribed from the gene for the jagged 1 set forth in SEQ ID No:393. In a preferred embodiment an mRNA transcribed from said gene comprises an mRNA target region having substantial homology to an mRNA target region set forth in SEQ ID No: 229.

The gene herein after referred to as “homeobox protein SHOTb” (or alternatively as SHOTB) is well-known in the art. (For a non-limiting example, see GenBank Accession AJ002368, herein incorporated by reference.) As used herein, the term “homeobox protein SHOTb” (as well as SHOTb) refers to a gene capable of transcribing an mRNA transcript having substantial homology with an mRNA transcribed from the gene for the homeobox protein SHOTb set forth in SEQ ID No:394. In a preferred embodiment an mRNA transcribed from said gene comprises an mRNA target region having substantial homology to an mRNA target region set forth in SEQ ID No: 230.

The gene herein after referred to as “death-associated protein kinase 3” (or alternatively as DAPK3) is well-known in the art. (For a non-limiting example, see GenBank Accession NM_{—}001348, herein incorporated by reference.) As used herein, the term “death-associated protein kinase 3” (as well as DAPK3) refers to a gene capable of transcribing an mRNA transcript having substantial homology with an mRNA transcribed from the gene for the death-associated protein kinase 3 set forth in SEQ ID No:395. In a preferred embodiment an mRNA transcribed from said gene comprises an mRNA target region having substantial homology to an mRNA target region set forth in SEQ ID No: 231.

The gene herein after referred to as “RAD51 homolog” (or alternatively as RecA homolog or RAD51) is well-known in the art. (For a non-limiting example, see GenBank Accession NM_{—}002875, herein incorporated by reference.) As used herein, the term D51 homolog” (as well as RecA homolog or RAD51) refers to a gene capable of transcribing an mRNA transcript having substantial homology with an mRNA transcribed from the gene for the RAD51 homolog (RecA homolog or RAD51) set forth in SEQ ID No:396. In a preferred embodiment an mRNA transcribed from said gene comprises an mRNA target region having substantial homology to an mRNA target region set forth in SEQ ID No: 232.

The gene herein after referred to as “methyl-CpG binding endonuclease” (or alternatively as MED1) is well-known in the art. (For a non-limiting example, see GenBank Accession AF114784, herein incorporated by reference.) As used herein, the term “methyl-CpG binding endonuclease” (as well as MED1) refers to a gene capable of transcribing an mRNA transcript having substantial homology with an mRNA transcribed from the gene for the methyl-CpG binding endonuclease set forth in SEQ ID No:397. In a preferred embodiment an mRNA transcribed from said gene comprises an mRNA target region having substantial homology to an mRNA target region set forth in SEQ ID No: 233.

The gene herein after referred to as “HUS1 checkpoint homolog” (or alternatively as HUS1) is well-known in the art. (For a non-limiting example, see GenBank Accession NM_{—}004507, herein incorporated by reference.) As used herein, the term “HUS1 checkpoint homolog” (as well as HUS1) refers to a gene capable of transcribing an mRNA transcript having substantial homology with an mRNA transcribed from the gene for the HUS1 checkpoint homolog set forth in SEQ ID No:398. In a preferred embodiment an mRNA transcribed from said gene comprises an mRNA target region having substantial homology to an mRNA target region set forth in SEQ ID No: 234.

The gene herein after referred to as “Human homolog of ES1” (or alternatively as HES1 (Y07572)) is well-known in the art. (For a non-limiting example, see GenBank Accession Y07572, herein incorporated by reference.) As used herein, the term “Human homolog of ES1” (as well as HES1 (Y07572)) refers to a gene capable of transcribing an mRNA transcript having substantial homology with an mRNA transcribed from the gene for Human homolog of ES1 set forth in SEQ ID No:399. In a preferred embodiment an mRNA transcribed from said gene comprises an mRNA target region having substantial homology to an mRNA target region set forth in SEQ ID No: 11.

The gene herein after referred to as “caldesmon 1” (or alternatively as CALDESMON or CALD1) is well-known in the art. (For a non-limiting example, see GenBank Accession NM_{—}033138, herein incorporated by reference.) As used herein, the term “caldesmon 1” (as well as CALDESMON or CALD1) refers to a gene capable of transcribing an mRNA transcript having substantial homology with an mRNA transcribed from the gene for the caldesmon 1 set forth in SEQ ID No:400. In a preferred embodiment an mRNA transcribed from said gene comprises an mRNA target region having substantial homology to an mRNA target region set forth in SEQ ID No: 235.

The gene herein after referred to as “VENT-like homeobox 2” (or alternatively as VENTX2) is well-known in the art. (For a non-limiting example, see GenBank Accession NM_{—}014468, herein incorporated by reference.) As used herein, the term “VENT-like homeobox 2” (as well as VENTX2) refers to a gene capable of transcribing an mRNA transcript having substantial homology with an mRNA transcribed from the gene for the VENT-like homeobox 2 set forth in SEQ ID No:401. In a preferred embodiment an mRNA transcribed from said gene comprises an mRNA target region having substantial homology to an mRNA target region set forth in SEQ ID No: 236.

The gene herein after referred to as “early growth response 2 protein” (or alternatively as EGR2) is well-known in the art. (For a non-limiting example, see GenBank Accession J04076, herein incorporated by reference.) As used herein, the term “early growth response 2 protein” (as well as EGR2) refers to a gene capable of transcribing an mRNA transcript having substantial homology with an mRNA transcribed from the gene for the early growth response 2 protein set forth in SEQ ID No:402. In a preferred embodiment an mRNA transcribed from said gene comprises an mRNA target region having substantial homology to an mRNA target region set forth in SEQ ID No: 237.

The gene herein after referred to as “Notch3” (or alternatively as NOTCH3) is well-known in the art. (For a non-limiting example, see GenBank Accession U97669, herein incorporated by reference.) As used herein, the term “Notch3” (as well as NOTCH3) refers to a gene capable of transcribing an mRNA transcript having substantial homology with an mRNA transcribed from the gene for the Notch3 set forth in SEQ ID No:403. In a preferred embodiment an mRNA transcribed from said gene comprises an mRNA target region having substantial homology to an mRNA target region set forth in SEQ ID No: 238.

The gene herein after referred to as “lin-28 homolog” (or alternatively as Lin28) is well-known in the art. (For a non-limiting example, see GenBank Accession NM_{—}024674, herein incorporated by reference.) As used herein, the term “lin-28 homolog” (as well as Lin28) refers to a gene capable of transcribing an mRNA transcript having substantial homology with an mRNA transcribed from the gene for the lin-28 homolog set forth in SEQ ID No:404. In a preferred embodiment an mRNA transcribed from said gene comprises an mRNA target region having substantial homology to an mRNA target region set forth in SEQ ID No: 239.

The gene herein after referred to as “PML-3” (or alternatively as PML3) is well-known in the art. (For a non-limiting example, see GenBank Accession M79464, herein incorporated by reference.) As used herein, the term “PML-3” (as well as PML3) refers to a gene capable of transcribing an mRNA transcript having substantial homology with an mRNA transcribed from the gene for the PML-3 set forth in SEQ ID No:405. In a preferred embodiment an mRNA transcribed from said gene comprises an mRNA target region having substantial homology to an mRNA target region set forth in SEQ ID No: 240.

The gene herein after referred to as “c-myc binding protein” (or alternatively as MYCBP) is well-known in the art. (For a non-limiting example, see GenBank Accession NM_{—}012333, herein incorporated by reference.) As used herein, the term “c-myc binding protein” (as well as MYCBP) refers to a gene capable of transcribing an mRNA transcript having substantial homology with an mRNA transcribed from the gene for the c-myc binding protein set forth in SEQ ID No:406. In a preferred embodiment an mRNA transcribed from said gene comprises an mRNA target region having substantial homology to an mRNA target region set forth in SEQ ID No: 241.

The gene herein after referred to as “transducer of ERBB2 1” (or alternatively as TOB1) is well-known in the art. (For a non-limiting example, see GenBank Accession NM_{—}005749, herein incorporated by reference.) As used herein, the term “transducer of ERBB2 1” (as well as TOB1) refers to a gene capable of transcribing an mRNA transcript having substantial homology with an mRNA transcribed from the gene for the transducer of ERBB2 1 set forth in SEQ ID No:407. In a preferred embodiment an mRNA transcribed from said gene comprises an mRNA target region having substantial homology to an mRNA target region set forth in SEQ ID No: 242.

The gene herein after referred to as “neuron navigator 3” (or alternatively as NAV3) is well-known in the art. (For a non-limiting example, see GenBank Accession NM_{—}014903, herein incorporated by reference.) As used herein, the term “neuron navigator 3” (as well as NAV3) refers to a gene capable of transcribing an mRNA transcript having substantial homology with an mRNA transcribed from the gene for the neuron navigator 3 set forth in SEQ ID No:408. In a preferred embodiment an mRNA transcribed from said gene comprises an mRNA target region having substantial homology to an mRNA target region set forth in SEQ ID No: 243.

The gene herein after referred to as “multiple asters 1” (or alternatively as MAST1) is well-known in the art. (For a non-limiting example, see GenBank Accession AF347693, herein incorporated by reference.) As used herein, the term “multiple asters 1” (as well as MAST1) refers to a gene capable of transcribing an mRNA transcript having substantial homology with an mRNA transcribed from the gene for the multiple asters 1 set forth in SEQ ID No:409. In a preferred embodiment an mRNA transcribed from said gene comprises an mRNA target region having substantial homology to an mRNA target region set forth in SEQ ID No: 244.

The gene herein after referred to as “headcase homolog” (or alternatively as HECA) is well-known in the art. (For a non-limiting example, see GenBank Accession NM_{—}016217, herein incorporated by reference.) As used herein, the term “headcase homolog” (as well as HECA) refers to a gene capable of transcribing an mRNA transcript having substantial homology with an mRNA transcribed from the gene for the headcase homolog set forth in SEQ ID No:410. In a preferred embodiment an mRNA transcribed from said gene comprises an mRNA target region having substantial homology to an mRNA target region set forth in SEQ ID No: 245.

The gene herein after referred to as “microtubule-associated protein 6” (or alternatively as MAP6) is well-known in the art. (For a non-limiting example, see GenBank Accession XM_{—}166256, herein incorporated by reference.) As used herein, the term “microtubule-associated protein 6” (as well as MAP6) refers to a gene capable of transcribing an mRNA transcript having substantial homology with an mRNA transcribed from the gene for the microtubule-associated protein 6 set forth in SEQ ID No:411. In a preferred embodiment an mRNA transcribed from said gene comprises an mRNA target region having substantial homology to an mRNA target region set forth in SEQ ID No: 246.

The gene herein after referred to as “methyl-CpG binding domain protein 1” (or alternatively as MBD1) is well-known in the art. (For a non-limiting example, see GenBank Accession NM_{—}015846, herein incorporated by reference.) As used herein, the term “methyl-CpG binding domain protein 1” (as well as MBD1) refers to a gene capable of transcribing an mRNA transcript having substantial homology with an mRNA transcribed from the gene for the methyl-CpG binding domain protein 1 set forth in SEQ ID No:412. In a preferred embodiment an mRNA transcribed from said gene comprises an mRNA target region having substantial homology to an mRNA target region set forth in SEQ ID No: 247.

The gene herein after referred to as “EphA5” (or alternatively as EPHA5) is well-known in the art. (For a non-limiting example, see GenBank Accession NM_{—}004439, herein incorporated by reference.) As used herein, the term “EphA5” (as well as EPHA5) refers to a gene capable of transcribing an mRNA transcript having substantial homology with an mRNA transcribed from the gene for the EphA5 set forth in SEQ ID No:413. In a preferred embodiment an mRNA transcribed from said gene comprises an mRNA target region having substantial homology to an mRNA target region set forth in SEQ ID No: 248.

The gene herein after referred to as “polymerase (RNA) III” (or alternatively as RPC32) is well-known in the art. (For a non-limiting example, see GenBank Accession NM_{—}006467, herein incorporated by reference.) As used herein, the term “polymerase (RNA) I” (as well as RPC32) refers to a gene capable of transcribing an mRNA transcript having substantial homology with an mRNA transcribed from the gene for the polymerase (RNA) m (DNA directed) set forth in SEQ ID No:414. In a preferred embodiment an mRNA transcribed from said gene comprises an mRNA target region having substantial homology to an mRNA target region set forth in SEQ ID No: 249.

The gene herein after referred to as “neuro-oncological ventral antigen 1” (or alternatively as NOVA1) is well-known in the art. (For a non-limiting example, see GenBank Accession NM_{—}002515, herein incorporated by reference.) As used herein, the term “neuro-oncological ventral antigen 1” (as well as NOVA1) refers to a gene capable of transcribing an mRNA transcript having substantial homology with an mRNA transcribed from the gene for the neuro-oncological ventral antigen 1 set forth in SEQ ID No:415. In a preferred embodiment an mRNA transcribed from said gene comprises an mRNA target region having substantial homology to an mRNA target region set forth in SEQ ID No: 250.

The gene herein after referred to as “activating transcription factor 1” (or alternatively as ATF1) is well-known in the art. (For a non-limiting example, see GenBank Accession NM_{—}005171, herein incorporated by reference.) As used herein, the term “activating transcription factor 1” (as well as ATF1) refers to a gene capable of transcribing an mRNA transcript having substantial homology with an mRNA transcribed from the gene for the activating transcription factor 1 set forth in SEQ ID No:416. In a preferred embodiment an mRNA transcribed from said gene comprises an mRNA target region having substantial homology to an mRNA target region set forth in SEQ ID No: 251.

The gene herein after referred to as “interphotoreceptor retinoid-binding protein” (or alternatively as IRBP) is well-known in the art. (For a non-limiting example, see GenBank Accession M22453, herein incorporated by reference.) As used herein, the term “interphotoreceptor retinoid-binding protein” (as well as IRBP) refers to a gene capable of transcribing an mRNA transcript having substantial homology with an mRNA transcribed from the gene for the interphotoreceptor retinoid-binding protein set forth in SEQ ID No:417. In a preferred embodiment an mRNA transcribed from said gene comprises an mRNA target region having substantial homology to an mRNA target region set forth in SEQ ID No: 252.

The gene herein after referred to as “E2F transcription factor 3” (or alternatively as E2F3) is well-known in the art. (For a non-limiting example, see GenBank Accession NM_{—}001949, herein incorporated by reference.) As used herein, the term “E2F transcription factor 3” (as well as E2F3) refers to a gene capable of transcribing an mRNA transcript having substantial homology with an mRNA transcribed from the gene for the E2F transcription factor 3 set forth in SEQ ID No:418. In a preferred embodiment an mRNA transcribed from said gene comprises an mRNA target region having substantial homology to an mRNA target region set forth in SEQ ID No: 253.

The gene herein after referred to as “mesoderm specific transcript homolog” (or alternatively as MEST) is well-known in the art. (For a non-limiting example, see GenBank Accession NM_{—}002402, herein incorporated by reference.) As used herein, the term “mesoderm specific transcript homolog” (as well as MEST) refers to a gene capable of transcribing an mRNA transcript having substantial homology with an mRNA transcribed from the gene for the mesoderm specific transcript homolog set forth in SEQ ID No:419. In a preferred embodiment an mRNA transcribed from said gene comprises an mRNA target region having substantial homology to an mRNA target region set forth in SEQ ID No: 254.

The gene herein after referred to as “bone morphogenetic protein 3” (or alternatively as BMP3) is well-known in the art. (For a non-limiting example, see GenBank Accession NM_{—}001201, herein incorporated by reference.) As used herein, the term “bone morphogenetic protein 3” (as well as BM3) refers to a gene capable of transcribing an mRNA transcript having substantial homology with an mRNA transcribed from the gene for the bone morphogenetic protein 3 (osteogenic) set forth in SEQ ID No:420. In a preferred embodiment an mRNA transcribed from said gene comprises an mRNA target region having substantial homology to an mRNA target region set forth in SEQ ID No: 255.

The gene herein after referred to as “EphA3” (or alternatively as EPHA3) is well-known in the art. (For a non-limiting example, see GenBank Accession NM_{—}005233, herein incorporated by reference.) As used herein, the term “EphA3” (as well as EPHA3) refers to a gene capable of transcribing an mRNA transcript having substantial homology with an mRNA transcribed from the gene for the EphA3 set forth in SEQ ID No:421. In a preferred embodiment an mRNA transcribed from said gene comprises an mRNA target region having substantial homology to an mRNA target region set forth in SEQ ID No: 256.

The gene herein after referred to as “methyl-CpG binding domain protein 6” (or alternatively as MBD5) is well-known in the art. (For a non-limiting example, see GenBank Accession NM_{—}018328, herein incorporated by reference.) As used herein, the term “methyl-CpG binding domain protein 6” (as well as MBD5) refers to a gene capable of transcribing an mRNA transcript having substantial homology with an mRNA transcribed from the gene for the methyl-CpG binding domain protein 5 set forth in SEQ ID No:422. In a preferred embodiment an mRNA transcribed from said gene comprises an mRNA target region having substantial homology to an mRNA target region set forth in SEQ ID No: 257.

The gene herein after referred to as “fibroblast growth factor 12” (or alternatively as FGF12) is well-known in the art. (For a non-limiting example, see GenBank Accession NM_{—}021032, herein incorporated by reference.) As used herein, the term “fibroblast growth factor 12” (as well as FGF12) refers to a gene capable of transcribing an mRNA transcript having substantial homology with an mRNA transcribed from the gene for the fibroblast growth factor 12 set forth in SEQ ID No:423. In a preferred embodiment an mRNA transcribed from said gene comprises an mRNA target region having substantial homology to an mRNA target region set forth in SEQ ID No: 258.

The gene herein after referred to as “RNA helicase A” (or alternatively as RNA helicase A) is well-known in the art. (or a non-limiting example, see GenBank Accession L13848, herein incorporated by reference.) As used herein, the term “RNA helicase A” (as well as RNA helicase A) refers to a gene capable of transcribing an mRNA transcript having substantial homology with an mRNA transcribed from the gene for the RNA helicase A set forth in SEQ ID No:424. In a preferred embodiment an mRNA transcribed from said gene comprises an mRNA target region having substantial homology to an mRNA target region set forth in SEQ ID No: 260.

The gene herein after referred to as “matrix metalloproteinase 26” (or alternatively as MMP26) is well-known in the art. (For a non-limiting example, see GenBank Accession NO₂₁₈₀₁, herein incorporated by reference.) As used herein, the term “matrix metalloproteinase 26” (as well as MMP26) refers to a gene capable of transcribing an mRNA transcript having substantial homology with an mRNA transcribed from the gene for the matrix metalloproteinase 26 set forth in SEQ ID No:425. In a preferred embodiment an mRNA transcribed from said gene comprises an mRNA target region having substantial homology to an mRNA target region set forth in SEQ ID No: 261.

The gene herein after referred to as “crossveinless-2” (or alternatively as Crossveinless-2) is well-known in the art. (For a non-limiting example, see GenBank Accession AY324650, herein incorporated by reference.) As used herein, the term “crossveinless-2” (as well as Crossveinless-2) refers to a gene capable of transcribing an mRNA transcript having substantial homology with an mRNA transcribed from the gene for the crossveinless-2 set forth in SEQ ID No:426. In a preferred embodiment an mRNA transcribed from said gene comprises an mRNA target region having substantial homology to an mRNA target region set forth in SEQ ID No: 262.

The gene herein after referred to as “cadherin 5 type 2 VE-cadherin” (or alternatively as CADHERIN5 or CDH5) is well-known in the art. (For a non-limiting example, see GenBank Accession NM_{—}001795, herein incorporated by reference.) As used herein, the term “cadherin 5 type 2 VE-cadherin” (as well as CADHERIN5 or CDH5) refers to a gene capable of transcribing an mRNA transcript having substantial homology with an mRNA transcribed from the gene for the cadherin 5 type 2 VE-cadherin (vascular epithelium) set forth in SEQ ID No:427. In a preferred embodiment an mRNA transcribed from said gene comprises an mRNA target region having substantial homology to an mRNA target region set forth in. SEQ ID No: 263.

The gene herein after referred to as “eukaryotic translation initiation factor 4A” (or alternatively as EIF4AI) is well-known in the art. (For a non-limiting example, see GenBank Accession NM_{—}001416, herein incorporated by reference.) As used herein, the term “eukaryotic translation initiation factor 4A” (as well as EIF4AI) refers to a gene capable of transcribing an mRNA transcript having substantial homology with an mRNA transcribed from the gene for the eukaryotic translation initiation factor 4A set forth in SEQ ID No:428. In a preferred embodiment an mRNA transcribed from said gene comprises an mRNA target region having substantial homology to an mRNA target region set forth in SEQ ID No: 264.

The gene herein after referred to as “TWEAK” (or alternatively as TWEAK) is well-known in the art. (For a non-limiting example, see GenBank Accession AF030099, herein incorporated by reference.) As used herein, the term “TWEAK” (as well as TWEAK) refers to a gene capable of transcribing an mRNA transcript having substantial homology with an mRNA transcribed from the gene for the TWEAK set forth in SEQ ID No:429. In a preferred embodiment an mRNA transcribed from said gene comprises an mRNA target region having substantial homology to an mRNA target region set forth in SEQ ID No: 265.

The gene herein after referred to as “fork head domain protein” (or alternatively as FKHR) is well-known in the art. (For a non-limiting example, see GenBank Accession U02310, herein incorporated by reference.) As used herein, the term “fork head domain protein” (as well as FKHR) refers to a gene capable of transcribing an mRNA transcript having substantial homology with an mRNA transcribed from the gene for the fork head domain protein set forth in SEQ ID No:430. In a preferred embodiment an mRNA transcribed from said gene comprises an mRNA target region having substantial homology to an mRNA target region set forth in SEQ ID No: 266.

The gene herein after referred to as “HOXB7” is well-known in the art. (For a non-limiting example, see GenBank Accession AJ414528, herein incorporated by reference.) As used herein, the term “HOXB7” refers to a gene capable of transcribing an mRNA transcript having substantial homology with an mRNA transcribed from the gene for the HOXB7 gene set forth in SEQ ID No:431. In a preferred embodiment an mRNA transcribed from said gene comprises an mRNA target region having substantial homology to an mRNA target region set forth in SEQ ID No: 268.

The gene herein after referred to as “Pax-3” is well-known in the art. (For a non-limiting example, see GenBank Accession AJ007392, herein incorporated by reference.) As used herein, the term “Pax-3” refers to a gene capable of transcribing an mRNA transcript having substantial homology with an mRNA transcribed from the gene for the Pax-3 set forth in SEQ ID No:432. In a preferred embodiment an mRNA transcribed from said gene comprises an mRNA target region having substantial homology to an mRNA target region set forth in SEQ ID No: 269.

The gene herein after referred to as “homeobox protein SHOTa” (or alternatively as SHOTa) is well-known in the art. (For a non-limiting example, see GenBank Accession AJ002367, herein incorporated by reference.) As used herein, the term “homeobox protein SHOTa” (as well as SHOTa) refers to a gene capable of transcribing an mRNA transcript having substantial homology with an mRNA transcribed from the gene for the homeobox protein SHOTa set forth in SEQ ID No:433. In a preferred embodiment an mRNA transcribed from said gene comprises an mRNA target region having substantial homology to an mRNA target region set forth in SEQ ID No: 270.

The gene herein after referred to as “inhibitor of growth family member 1” (or alternatively as ING1) is well-known in the art. (For a non-limiting example, see GenBank Accession NM_{—}198219, herein incorporated by reference.) As used herein, the term “inhibitor of growth family member 1” (as well as ING1) refers to a gene capable of transcribing an mRNA transcript having substantial homology with an mRNA transcribed from the gene for the inhibitor of growth family member 1 set forth in SEQ ID No:434. In a preferred embodiment an mRNA transcribed from said gene comprises an mRNA target region having substantial homology to an mRNA target region set forth in SEQ ID No: 271.

The gene herein after referred to as “v-ets erythroblastosis virus E26 oncogene like” (or alternatively as V-ETS or ERG) is well-known in the art. (For a non-limiting example, see GenBank Accession NM_{—}004449, herein incorporated by reference.) As used herein, the term “v-ets erythroblastosis virus E26 oncogene like” (as well as V-ETS or ERG) refers to a gene capable of transcribing an mRNA transcript having substantial homology with an mRNA transcribed from the gene for the v-ets erythroblastosis virus E26 oncogene like set forth in SEQ ID No:435. In a preferred embodiment an m-RNA transcribed from said gene comprises an mRNA target region having substantial homology to an mRNA target region set forth in SEQ ID No: 272.

The gene herein after referred to as “reticulon 4′ (or alternatively as RTN4) is well-known in the art. (For a non-limiting example, see GenBank Accession NM_{—}020532, herein incorporated by reference.) As used herein, the term “reticulon 4” (as well as RTN4) refers to a gene capable of transcribing an mRNA transcript having substantial homology with an mRNA transcribed from the gene for the reticulon 4 set forth in SEQ ID No:436. In a preferred embodiment an mRNA transcribed from said gene comprises an miRNA target region having substantial homology to an miRNA target region set forth in SEQ ID No: 273.

The gene herein after referred to as “NOD2 protein” (or alternatively as NOD2) is well-known in the art. (For a non-limiting example, see GenBank Accession AF178930, herein incorporated by reference.) As used herein, the term LNOD2 protein” (as well as NOD2) refers to a gene capable of transcribing an mRNA transcript having substantial homology with an mRNA transcribed from the gene for the NOD2 protein set forth in SEQ ID No:437. In a preferred embodiment an mRNA transcribed from said gene comprises an mRNA target region having substantial homology to an mRNA target region set forth in SEQ ID No: 274.

The gene herein after referred to as “interleukin 6 receptor” (or alternatively as IL6R) is well-known in the art. (For a non-limiting example, see GenBank Accession NM_{—}000565, herein incorporated by reference.) As used herein, the term “interleukin 6 receptor” (as well as IL6R) refers to a gene capable of transcribing an mRNA transcript having substantial homology with an mRNA transcribed from the gene for the interleukin 6 receptor set forth in SEQ ID No:438. In a preferred embodiment an mRNA transcribed from said gene comprises an mRNA target region having substantial homology to an mRNA target region set forth in SEQ ID No: 275.

The gene herein after referred to as “PML-2 mRNA” (or alternatively as PML2) is well-known in the art. (For a non-limiting example, see GenBank Accession M79463, herein incorporated by reference.) As used herein, the term “PML-2 mRNA” (as well as PML2) refers to a gene capable of transcribing an mRNA transcript having substantial homology with an mRNA transcribed from the gene for the PML-2 mRNA set forth in SEQ ID No:439. In a preferred embodiment an mRNA transcribed from said gene comprises an mRNA target region having substantial homology to an miRNA target region set forth in SEQ ID No: 276.

The gene herein after referred to as “discs large homolog 1” (or alternatively as DLG1) is well-known in the art. (For a non-limiting example, see GenBank Accession NM_{—}004087, herein incorporated by reference.) As used herein, the term “discs large homolog 1” (as well as DLG1) refers to a gene capable of transcribing an mRNA transcript having substantial homology with an mRNA transcribed from the gene for the discs large homolog 1 set forth in SEQ ID No:440. In a preferred embodiment an mRNA transcribed from said gene comprises an mRNA target region having substantial homology to an mRNA target region set forth in SEQ ID No: 277.

The gene herein after referred to as “Yes-associated protein 1” (or alternatively as YAP1) is well-known in the art. (For a non-limiting example, see GenBank Accession NM_{—}006106, herein incorporated by reference.) As used herein, the term “Yes-associated protein 1” (as well as YAP1) refers to a gene capable of transcribing an mRNA transcript having substantial homology with an mRNA transcribed from the gene for the Yes-associated protein 1 set forth in SEQ ID No:441. In a preferred embodiment an mRNA transcribed from said gene comprises an mRNA target region having substantial homology to an mRNA target region set forth in SEQ ID No: 278.

The gene herein after referred to as “CD 14 antigen” (or alternatively as CD 14) is well-known in the art. (For a non-limiting example, see GenBank Accession NM_{—}000591, herein incorporated by reference.) As used herein, the term “CD14 antigen” (as well as CD 14) refers to a gene capable of transcribing an mRNA transcript having substantial homology with an mRNA transcribed from the gene for the CD14 antigen set forth in SEQ ID No:442. In a preferred embodiment an mRNA transcribed from said gene comprises an mRNA target region having substantial homology to an mRNA target region set forth in SEQ ID No: 279.

The gene herein after referred to as “negative differentiation regulator” (or alternatively as NDR) is well-known in the art. (For a non-limiting example, see GenBank Accession AY255672, herein incorporated by reference.) As used herein, the term “negative differentiation regulator” (as well as NDR) refers to a gene capable of transcribing an mRNA transcript having substantial homology with an mRNA transcribed from the gene for the negative differentiation regulator set forth in SEQ ID No:443. In a preferred embodiment an mRNA transcribed from said gene comprises an mRNA target region having substantial homology to an mRNA target region set forth in SEQ ID No: 280.

The gene herein after referred to as “CREB binding protein” (or alternatively as CBP or CREBBP) is well-known in the art. (For a non-limiting example, see GenBank Accession NM_{—}004380, herein incorporated by reference.) As used herein, the term “CREB binding protein” (as well as CBP or CREBBP) refers to a gene capable of transcribing an mRNA transcript having substantial homology with an mRNA transcribed from the gene for the CREB binding protein (Rubinstein-Taybi syndrome) set forth in SEQ ID No:444. In a preferred embodiment an mRNA transcribed from said gene comprises an mRNA target region having substantial homology to an mRNA target region set forth in SEQ ID No: 281.

The gene herein after referred to as “v-ski sarcoma viral oncogene homolog” (or alternatively as V-ski or SKI) is well-known in the art. (For a non-limiting example, see GenBank Accession N_{—}003036, herein incorporated by reference.) As used herein, the term “v-ski sarcoma viral oncogene homolog” (as well as V-ski or SKI) refers to a gene capable of transcribing an mRNA transcript having substantial homology with an mRNA transcribed from the gene for the v-ski sarcoma viral oncogene homolog set forth in SEQ ID No:445. In a preferred embodiment an mRNA transcribed from said gene comprises an mRNA target region having substantial homology to an mRNA target region set forth in SEQ ID No: 282.

The gene herein after referred to as “sidekick homolog 1” (or alternatively as SDK1) is well-known in the art. (For a non-limiting example, see GenBank Accession NM_{—}152744, herein incorporated by reference.) As used herein, the term “sidekick homolog 1” (as well as SDK1) refers to a gene capable of transcribing an mRNA transcript having substantial homology with an mRNA transcribed from the gene for the sidekick homolog 1 set forth in SEQ ID No:446. In a preferred embodiment an mRNA transcribed from said gene comprises an mRNA target region having substantial homology to an mRNA target region set forth in SEQ ID No: 283.

The gene herein after referred to as “bone morphogenetic protein receptor type II” (or alternatively as BMPR2) is well-known in the art. (For a non-limiting example, see GenBank Accession NM_{—}001204, herein incorporated by reference.) As used herein, the term “bone morphogenetic protein receptor type II” (as well as BMPR2) refers to a gene capable of transcribing an mRNA transcript having substantial homology with an mRNA transcribed from the gene for the bone morphogenetic protein receptor type II set forth in SEQ ID No:447. In a preferred embodiment an mRNA transcribed from said gene comprises an mRNA target region having substantial homology to an mRNA target region set forth in SEQ ID No: 284.

The gene herein after referred to as “programmed cell death 10” (or alternatively as PDCD10) is well-known in the art. (For a non-limiting example, see GenBank Accession NM_{—}007217, herein incorporated by reference.) As used herein, the term “programmed cell death 10” (as well as PDCD10) refers to a gene capable of transcribing an mRNA transcript having substantial homology with an mRNA transcribed from the gene for the programmed cell death 10 set forth in SEQ ID No:448. In a preferred embodiment an mRNA transcribed from said gene comprises an mRNA target region having substantial homology to an mRNA target region set forth in SEQ ID No: 285.

The gene herein after referred to as “cyclin H” (or alternatively as CDK7 or CCNH) is well-known in the art. (For a non-limiting example, see GenBank Accession NM_{—}001239, herein incorporated by reference.) As used herein, the term “cyclin H” (as well as CDK7 or CCNH refers to a gene capable of transcribing an mRNA transcript having substantial homology with an mRNA transcribed from the gene for the cyclin H set forth in SEQ ID No:449. In a preferred embodiment an mRNA transcribed from said gene comprises an mRNA target region having substantial homology to an mRNA target region set forth in SEQ ID No: 286.

The gene herein after referred to as “nuclear protein double minute 1” (or alternatively as MDM1) is well-known in the art. (For a non-limiting example, see GenBank Accession NM_{—}017440, herein incorporated by reference.) As used herein, the term “nuclear protein double minute 1” (as well as MDM1) refers to a gene capable of transcribing an mRNA transcript having substantial homology with an mRNA transcribed from the gene for the nuclear protein double minute 1 set forth in SEQ ID No:450. In a preferred embodiment an mRNA transcribed from said gene comprises an mRNA target region having substantial homology to an mRNA target region set forth in SEQ ID No: 287.

The gene herein after referred to as “BCL2/adenovirus E1B 19 kDa interacting protein 2” (or alternatively as BNIP2) is well-known in the art. (For a non-limiting example, see GenBank Accession NM_{—}004330, herein incorporated by reference.) As used herein, the term “BCL2/adenovirus EIB 19 kDa interacting protein 2” (as well as BNIP2) refers to a gene capable of transcribing an mRNA transcript having substantial homology with an mRNA transcribed from the gene for the BCL2/adenovirus E1B 19 kDa interacting protein 2 set forth in SEQ ID No:451. In a preferred embodiment an mRNA transcribed from said gene comprises an mRNA target region having substantial homology to an mRNA target region set forth in SEQ ID No: 288.

The gene herein after referred to as “karyopherin (importin) beta 2” (or alternatively as Importin beta2) is well-known in the art. (For a non-limiting example, see GenBank Accession BC040340, herein incorporated by reference.) As used herein, the term “karyopherin (importin) beta 2” (as well as Importin beta2) refers to a gene capable of transcribing an mRNA transcript having substantial homology with an mRNA transcribed from the gene for the karyopherin (importin) beta 2 set forth in SEQ ID No:452. In a preferred embodiment an mRNA transcribed from said gene comprises an mRNA target region having substantial homology to an mRNA target region set forth in SEQ ID No: 289.

The gene herein after referred to as “v-ros UR2 sarcoma virus oncogene homolog 1” (or alternatively as V-ros or ROS1) is well-known in the art. (For a non-limiting example, see GenBank Accession NM_{—}002944, herein incorporated by reference.) As used herein, the term “v-ros UR2 sarcoma virus oncogene homolog 1” (as well as V-ros or ROS1) refers to a gene capable of transcribing an mRNA transcript having substantial homology with an mRNA transcribed from the gene for the v-ros UR2 sarcoma virus oncogene homolog 1 set forth in SEQ ID No:453. In a preferred embodiment an mRNA transcribed from said gene comprises an mRNA target region having substantial homology to an mRNA target region set forth in SEQ ID No: 290.

As disclosed herein, 100% sequence identity between the RNA and the target gene is not required to practice the present invention. Indeed, among the example of the instant invention, mRNAs having identities sharing as littles as about 50% identity with a corresponding mRNA target region hve been found to effectively modulate expression of a target gene. Thus, RNAs of the invention have the advantage of being able to tolerate sequence variations that might be expected due to genetic mutation, strain polymorphism, or evolutionary divergence.

The present invention provides products and methods for modulating expression of a target gene in a cell. One such method comprises introducing into the cell a polynucleotide that forms a duplex region with an mRNA transcribed from said target gene, wherein the duplex region comprises a mammalian mRNA target region. Another such method comprises introducing into the cell an siRNA that forms a duplex region with an mRNA, or precursor thereof, wherein an mRNA transcribed from the target gene comprises a mRNA target region. In certain preferred embodiments, the methods further comprise measuring expression of the target gene. The method are particularly useful for modulating ontogenesis, function, differentiation and/or viability of a mammalian cell. As such, the invention also provides methods for controlling ontogenesis of mammal, function of mammalian cell, differentiation of mammalian cell or viability of mammalian cell in the post-transcriptional phase by introducing into the cell a miRNA or a siRNA silencing precursor to the miRNA.

In one embodiment, the invention provides a method for modulating expression of a target gene in a cell, the method comprising introducing into the cell a polynucleotide that forms a duplex region with an mRNA transcribed from said target gene, wherein said duplex region comprises a mammalian miRNA target region. In a preferred embodiment, the mRNA target region comprises a sequence having at least about 70% identity to a polynucleotide selected from SEQ ID Nos: 5-11, 13, and 121-290. In more preferred embodiments, the miRNA target region comprises a sequence having at least about 80% identity, at least about 90% identity, or at least about 95% identity to a polynucleotide selected from SEQ ID Nos: 5-11, 13, and 121-290. In certain preferred embodiments, the inventive methods employ an mRNA or a precursor thereof, or a vector encoding said mRNA or a precursor thereof for use as a polynucleotide to be introduced into the cell. In certain preferred embodiments, the cell is a mammalian cell, and preferably a human cell. The cell may be an isolated cell or may be part of a culture, tissue, or whole multi-cellular organism. In a preferred embodiment, the mRNA comprises a sequence having at least about 70% identity to a polynucleotide selected from SEQ ID Nos: 1, 3, 12, and 14-120. In more preferred embodiments, the mRNA comprises a sequence having at least about 80% identity, at least about 90% identity, or at least about 95% identity to a polynucleotide selected from selected from SEQ ID Nos: 1, 3, 12, and 14-120.

Particularly preferred embodiments of mRNAs for use in the inventive method include miR-1, miR-2-1, miR-5, miR-7, miR-8, miR-11, miR-12, miR-13, miR-14, miR-16, miR-16, miR-17, miR-18, miR-19, miR-20, miR-21, miR-22, miR-23, miR-24, miR-25, miR-26, miR-27, miR-28, miR-29, miR-30, miR-31, miR-32, miR-33, miR-34, miR-92, miR-93, miR-94, miR-95, miR-96, miR-97, miR-98, miR-99, miR-100, miR101, miR-103, miR-104, miR-105, miR-106, miR-107, miR-109, miR-110, miR-111, miR-112, miR-113, miR-114, miR-116, miR-119, miR-122, miR-125, miR-126, miR-127, miR-129, miR-130, miR-132, miR-133, miR-134, miR-136, miR-138, miR-140, miR-141, miR-144, miR-145, miR-146, miR-147, miR-148, miR-149, miR-150, miR-151, miR-153, miR-154, miR-157, miR-158, miR-160, miR-162, miR-164, miR-172, miR-173, miR-174, miR-175, miR-176, miR-177, miR-178, miR-179, miR-180, miR-182, miR-183, miR-184, miR-185, miR-186, miR-187, miR-188, miR-189, miR-191, miR-192, miR-193, miR-195, miR-196, miR-197, miR-199, miR-201, miR-203, miR-205, and miR-224, or a precursor thereof.

Preferred embodiments of the inventive methods include those for modulating expression of a target gene in a cell, wherein the target gene is one or more of dbl proto-oncogene; transforming growth factor beta 1; transforming growth factor alpha; v-myb myeloblastosis viral oncogene homolog; c-cbl proto-oncogene; snoI; activin beta E subunit; myogenic factor 5; fibroblast growth factor 9; RON encoding a tyrosine kinase; E3 ubiquitin ligase SMURF1; jagged 2; jun-B encoding the JUN-B protein; methyl-CpG binding domain protein 4; ZIP kinase; endomucin; ICE-protease activating factor; hairy and enhancer of split 1; transforming growth factor beta 3; enaptin mRNA; AMP deaminase; interleukin 1 alpha; E2F transcription factor 6; laminin alpha; polymerase (DNA-directed) alpha; leukocyte tyrosine kinase; homeo box D1; laminin gamma; tumor necrosis factor receptor superfamily member 1A; villin 2; frizzled homolog 5; ATP-dependent chromatin remodelling protein; MSX2 mRNA for transcription factor; adipose differentiation-related protein; myogenic factor 4; SRY (Sex determining Region Y)-box 5; Notch homolog 1; Human tyrosine kinase-type receptor; polymerase (DNA directed) theta; cAMP responsive element binding protein 3; timeless homolog; RAD52 homolog; toll-like receptor 4; SRY (Sex determining Region Y)-box 9; homeo box A5; cell division cycle 42 GTP binding protein; desmuslin; TFIIIC Box B-binding subunit; profilin 2; c-fms proto-oncogene; delta-like 1; fatty-acid-Coenzyme A ligase long-chain 5; discs large homolog-associated protein 2; TFIIH gene for transcription factor II H; RNA polymerase III subunit RPC; RecQ protein-like 5; METH2 protein; MOST2 protein; SRY (Sex determining Region Y)-box 7; integrin beta 1 subunit; desmin; protection of telomeres 1; H2.0-like homeo box 1; GABA transport protein; v-myc myelocytomatosis viral related oncogene neuroblastoma derived; BAG-family molecular chaperone regulator-S; Human placental bone morphogenic protein; retinoblastoma-associated factor 600; ALK-4; tolloid-like 2; RIGB; Human DNA repair helicase; T-box 22; BRCA1 associated protein 1; Sp3 transcription factor; TEF-1 gene; forkhead box A3; ets family transcription factor ELF2A; microtubule-associated protein 1A; myosin 5B; NEDD4-like ubiquitin ligase 1; Mint1 mRNA; PARX protein; epidermal growth factor receptor; matrix metalloproteinase 3; VE-cadherin; microtubule-associated protein 2; TAF7 RNA polymerase II TATA box binding protein (TBP)-associated factor; mitochondrial elongation factor G2; eyes absent homolog; paired box gene 3; synaptotagmin I; histone deacetylase 5; homolog of Drosophila headcase; homeo box B8; fyn-related kinase; TGF-beta/activin signal transducer FAST-1p; La autoantigen; mutL homolog 1; E74-like factor 3; B-myb gene; a-myb mRNA; jagged 1; homeobox protein SHOTb; death-associated protein kinase 3; RAD51 homolog (RecA homolog); methyl-CpG binding endonuclease; HUS1 checkpoint homolog; HES1 protein; caldesmon 1; VENT-like homeobox 2; early growth response 2 protein; Notch3; lin-28 homolog; PML-3; c-myc binding protein; transducer of ERBB2 1; neuron navigator 3; multiple asters 1; headcase homolog; microtubule-associated protein 6; methyl-CpG binding domain protein 1; EphA6; polymerase (RNA) II (cDNA directed); neuro-oncological ventral antigen 1; activating transcription factor 1; interphotoreceptor retinoid-binding protein; E2F transcription factor 3; mesoderm specific transcript homolog; bone morphogenetic protein 3; EphA3; methyl-CpG binding domain protein 5; fibroblast growth factor 12; RNA helicase A; matrix metalloproteinase 26; crossveinless-2; cadherin 5 type 2 VE-cadherin; eukaryotic translation initiation factor 4A; TWEAK; fork head domain protein; HOXB7 gene; Pax-3; homeobox protein SHOTa; inhibitor of growth family member 1; v-ets erythroblastosis virus E26 oncogene like; reticulon 4; NOD2 protein; interleukin 6 receptor; PML-2 mRNA; discs large homolog 1; Yes-associated protein 1; CD14 antigen; negative differentiation regulator; CREB binding protein; v-ski sarcoma viral oncogene homolog; sidekick homolog 1; bone morphogenetic protein receptor type II; programmed cell death 10; cyclin H; nuclear protein double minute 1; BCL2/adenovirus E1B 19 kDa interacting protein 2; karyopherin beta 2; and v-ros UR2 sarcoma virus oncogene homolog 1.

In a preferred embodiment, the gene target of the method comprises a polynucleotide sequence that hybridizes under moderately stringent conditions with a polynucleotide sequence selected from SEQ ID Nos: 291-454.

It is particularly preferred that the mRNA transcribed from said target gene comprises a polynucleotide sequence having at least 70% identity to a polynucleotide selected from SEQ ID Nos: 5-11, 13, and 121-290. However, the method contemplates mRNA molecules having mRNA target sequences other than those set forth in 5-11, 13, and 121-290.

In another inventive method for modulating expression of a mammalian target gene in a cell contemplated by the instant invention, an siRNA that forms a duplex region with an mRNA, or precursor thereof is introduced into a cell comprising an mRNA transcribed from a target gene, where the target gene comprises an mRNA target region. In preferred embodiments of the inventive method, the siRNA forms a duplex region with an mRNA. The resulting duplex may result in, for example, inhibiting the mRNA from forming a second duplex region with mRNA transcribed from said target gene. It is particularly preferred that the siRNA forms a duplex region with an mRNA precursor, thereby inhibiting the mRNA precursor from converting to mRNA.

In certain embodiments, the mRNA or precursor thereof comprises a sequence having at least about 70% identity to a polynucleotide selected from SEQ ID Nos: 1, 3, 12, and 14-120. Among the especially preferred embodiments are those in which wherein the mRNA target region of the method comprises a sequence having at least about 70% identity to a polynucleotide selected from SEQ ID Nos: 5-11, 13, and 121-290.

Contemplated methods include those in which the siRNA target is one or more of miR-1, miR-2-1, miR-5, miR-7, miR-8, miR-11, miR-12, miR-13, miR-14, miR-15, miR-16, miR-17, miR-18, miR-19, miR-20, miR-21, miR-22, miR-23, miR-24, miR-25, miR-26, miR-27, miR-28, miR-29, miR-30, miR-31, miR-32, miR-33, miR-34, miR-92, miR-93, miR-94, miR-95, miR-96, miR-97, miR-98, miR-99, miR-100, miR101, miR-103, miR-104, miR-105, miR-106, miR-107, miR-109, miR-110, miR-111, miR-112, miR-113, miR-114, miR-116, miR-119, miR-122, miR-125, miR-126, miR-127, miR-129, miR-130, miR-132, miR-133, miR-134, miR-136, miR-138, miR-140, miR-141, miR-144, miR-145, miR-146, miR-147, miR-148, miR-149, miR-150, miR-161, miR-163, miR-154, miR-157, miR-158, miR-160, miR-162, miR-164, miR-172, miR-173, miR-174, miR-175, miR-176, miR-177, miR-178, miR-179, miR-180, miR-182, miR-183, miR-184, miR-185, miR-186, miR-187, miR-188, miR-189, miR-191, miR-192, miR-193, miR-195, miR-196, miR-197, miR-199, miR-201, miR-203, miR-205, and miR-224, or a precursor thereof. mRNA or precursor thereof comprising a sequence selected from the group consisting of SEQ ID Nos: 1, 3, 12, and 14-120 is especially preferred.

In certain embodiments of the inventive methods employing an siRNA that forms a duplex region with an mRNA, or precursor thereof, a preferred target gene is includes one or more of: dbl proto-oncogene; transforming growth factor beta 1; transforming growth factor alpha; v-myb myeloblastosis viral oncogene homolog; c-cbl proto-oncogene; snoI; activin beta E subunit; myogenic factor 5; fibroblast growth factor 9; RON encoding a tyrosine kinase; E3 ubiquitin ligase SMURF1; jagged 2; jun-B encoding the JUN-B protein; methyl-CpG binding domain protein 4; ZIP kinase; endomucin; ICE-protease activating factor; hairy and enhancer of split 1; transforming growth factor beta 3; enaptin mRNA; AMP deaminase; interleukin 1 alpha; E2F transcription factor 6; laminin alpha; polymerase (DNA-directed) alpha; leukocyte tyrosine kinase; homeo box D1; laminin gamma; tumor necrosis factor receptor superfamily member 1A; villin 2; frizzled homolog 5; ATP-dependent chromatin remodelling protein; MSX2 mRNA for transcription factor; adipose differentiation-related protein; myogenic factor 4; SRY (Sex determining Region Y)-box 5; Notch homolog 1; Human tyrosine kinase-type receptor; polymerase (DNA directed) theta; cAMP responsive element binding protein 3; timeless homolog; RAD52 homolog; toll-like receptor 4; SRY (Sex determining Region Y)-box 9; homeo box As; cell division cycle 42 GTP binding protein; desmuslin; TFIIIC Box B-binding subunit; profilin 2; c-fms proto-oncogene; delta-like 1; fatty-acid-Coenzyme A ligase long-chain 5; discs large homolog-associated protein 2; TFIIH gene for transcription factor II H; RNA polymerase III subunit RPC; RecQ protein-like 5; METH2 protein; MOST2 protein; SRY (Sex determining Region Y)-box 7; integrin beta 1 subunit; desmin; protection of telomeres 1; H2.0-like homeo box 1; GABA transport protein; v-myc myelocytomatosis viral related oncogene neuroblastoma derived; BAG-family molecular chaperone regulator-S; Human placental bone morphogenic protein; retinoblastoma-associated factor 600; ALK-4; tolloid-like 2; RIGB; Human DNA repair helicase; T-box 22; BRCA1 associated protein 1; Sp3 transcription factor; TEF-1 gene; forkhead box A3; ets family transcription factor ELF2A; microtubule-associated protein 1A; myosin 5B; NEDD4-like ubiquitin ligase 1; Mint1 mRNA; PARX protein; epidermal growth factor receptor; matrix metalloproteinase 3; VE-cadherin; microtubule-associated protein 2; TAF7 RNApolymerase II TATA box binding protein (TBP)-associated factor; mitochondrial elongation factor G2; eyes absent homolog; paired box gene 3; synaptotagmin I; histone deacetylase 5; homolog of Drosophila headcase; homeo box B8; fyn-related kinase; TGF-beta/activin signal transducer FAST-1p; La autoantigen; mutL homolog 1; E74-like factor 3; B-myb gene; a-myb mRNA; jagged 1; homeobox protein SHOTb; death-associated protein kinase 3; RAD51 homolog (RecA homolog); methyl-CpG binding endonuclease; HUS1 checkpoint homolog; HES1 protein; caldesmon 1; VENT-like homeobox 2; early growth response 2 protein; Notch3; lin-28 homolog; PML-3; c-myc binding protein; transducer of ERBB2 1; neuron navigator 3; multiple asters 1; headcase homolog; microtubule-associated protein 6; methyl-CpG binding domain protein 1; EphA5; polymerase (RNA) III (DNA directed); neuro-oncological ventral antigen 1; activating transcription factor 1; interphotoreceptor retinoid-binding-protein; E2F transcription factor 3; mesoderm specific transcript homolog; bone morphogenetic protein 3; EphA3; methyl-CpG binding domain protein 5; fibroblast growth factor 12; RNA helicase A; matrix metalloproteinase 26; crossveinless-2; cadherin 5 type 2 VE-cadherin; eukaryotic translation initiation factor 4A TWEAK; fork head domain protein; HOXB7 gene; Pax-3; homeobox protein SHOTa; inhibitor of growth family member 1; v-ets erythroblastosis virus E26 oncogene like; reticulon 4; NOD2 protein; interleukin 6 receptor; PML-2 mRNA; discs large homolog 1; Yes-associated protein 1; CD14 antigen; negative differentiation regulator; CREB binding protein; v-ski sarcoma viral oncogene homolog; sidekick homolog 1; bone morphogenetic protein receptor type II; programmed cell death 10; cyclin H; nuclear protein double minute 1; BCL2/adenovirus E1B 19 kDa interacting protein 2; karyopherin beta 2; and v-roe UR2 sarcoma virus oncogene homolog 1.

Preferred embodiments include those in which the target gene comprises a polynucleotide sequence that hybridizes under moderately stringent conditions with a polynucleotide sequence selected from SEQ ID Nos: 291-454. It is especially preferred that the mRNA transcribed from the target gene comprises a polynucleotide sequence having at least about 70% identity to a polynucleotide selected from SEQ ID Nos: 1, 3, 12, and 14-120.

The methods of the invention may additionally comprise measuring expression of said target gene.

As one of skill in the art would recognize, the inventive methods of the application may be employed to accomplish a variety of objectives. For example, the methods may be used to modulate ontogenesis, function, differentiation and/or viability of a mammalian cell. As such, the invention contemplates methods for controlling ontogenesis of mammal, function of mammalian cell, differentiation of mammalian cell or viability of mammalian cell in the post-transcriptional phase, the methods comprising introducing into the cell a mRNA or an siRNA silencing precursor to an endogenous or heterologous mRNA. By way of example, methods of the instant invention may be employed to control differentiation of nerve cell by regulating expression of hairy and enhancer of split 1.

Where an siRNA is introduced into the cell, one preferred embodiment contemplates the siRNA binding to a loop in stem-loop structure of an mRNA or precursor thereof. In such methods, it is preferred that siRNA has a sequence with at least about 70% identity to the sequence disclosed in SEQ ID No: 2. However, siRNAs prepared to to target other mRNA are also contemplated. Contemplated targets include, for example, miR-1, miR-2-1, miR-5, miR-7, miR-8, miR-11, miR-12, miR-13, miR-14, miR-15, miR-16, miR-17, miR-18, miR-19, miR-20, miR-21, miR-22, miR-23, miR-24, miR-25, miR-26, miR-27, miR-28, miR-29, miR-30, miR-31, miR-32, miR-33, miR-34, miR-92, miR-93, miR-94, miR-95, miR-96, miR-97, miR-98, miR-99, miR-100, miR101, miR-103, miR-104, miR-105, miR-106, miR-107, miR-109, miR-110, miR-111, miR-112, miR-113, miR-114, miR-116, miR-119, miR-122, miR-125, miR-126, miR-127, miR-129, miR-130, miR-132, miR 133, miR-134, miR-136, miR-138, miR-140, miR-141, miR-144, miR-145, miR-146, miR-147, miR-148, miR-149, miR-150, miR-151, miR-153, miR-154, miR-157, miR-158, miR-160, miR-162, miR-164, miR-172, miR-173, miR-174, miR-175, miR-176, miR-177, miR-178, miR-179, miR-180, miR-182, miR-183, miR-184, miR-185, miR-186, miR-187, miR-188, miR-189, miR-191, miR-192, miR-193, miR-195, miR-196, miR-197, miR-199, miR-201, miR-203, miR-205, and miR-224, and precursors thereof. Furthermore, it is preferred that siRNA has a sequence with at least about have substantial homology with a contemplated target sequence found in an mRNA or precursor thereof.

The invention further contemplates plasmid vectors comprising a promoter and a polynucleotide sequence expressing mRNA or a precursor to the mRNA. Also contemplated are plasmid vectors comprising a promoter and a nucleotide sequence expressing siRNA silencing precursor to mRNA. With respect to vectors encoding siRNA, it is especially preferred that such vectors encode mRNA that is capable of forming a duplex region with an mRNA transcribed from a mammalian target gene. Promoters selected from the group consisting of tRNA^{(val)} promoter, U6 promoter, H1 promoter and Pol II promoter, such as CMV and SV40, are especially preferred.

The invention contemplates methods employing the use of the contemplated vectors for controlling ontogenesis of mammal, function of mammalian cell, differentiation of mammalian cell or viability of mammalian, the methods comprising introducing into the cell a contemplated plasmid vector.

Furthermore, the invention contemplates methods for treating cancer, immune disease, nerve disorder or inflammatory disease, the methods comprising introducing into a cell an mRNA, a siRNA silencing precursor to the mRNA or the plasmid vector as described herein. A particularly preferred method comprises treating a nerve disorder selected from amyotrophic lateral sclerosis (ALS), Parkinson disease or Alzheimer disease.

The invention provides for methods useful in screening pharmaceuticals using an mRNA, an siRNA silencing precursor to the mRNA or the plasmid vector defined, the methods employing the vectors as described herein. It is particularly preferred that the target mRNA is derived from a recombinant gene having a sequence of the target region of the mRNA.

Especially preferred methods are those for gene function analysis using a mRNA, a siRNA silencing precursor to the mRNA or the plasmid vector defined as described herein. Other preferred methods include those for regulation of cell differentiation to muscle cell, bone cell or myocardial cell, where the gene to be regulated is a gene whose function is identified by the gene function analysis as described herein. Also contemplated are methods for preservation or maintenance of anaplastic cell, introducing into cell a substance suppressing expression of miR-23; methods for regulating ratio of gene expression, by producing recombinant of selected gene and target sequence of miR-23 of Hes1, and designing miR-23 sequence 50 to 90% complementary to the target sequence; methods for suppressing gene expression, the method comprising introducing into cell an siRNA inducing decomposition of mRNA and a mRNA, such as, for example, miR-23.

### EXAMPLES

The invention is further described by example. The examples, however, are provided for purposes of illustration to those skilled in the art, and are not intended to be limiting. Moreover, the examples are not to be construed as limiting the scope of the appended claims. Thus, the invention should in no way be construed as being limited to the following examples, but rather, should be construed to encompass any and all variations that become evident as a result of the teaching provided herein.

Hea1 is a Target of miR-23 in NT2 Cells

It has been reported that some of the Drosophila mRNAs that align to the K box motif (5′-UGUGAU-3′) mediate a negative post-transcriptional regulation of the Hairy/enhancer of split (HEEL gene family in Drosophila2830. A human miR-23 containing the antisense sequence to the K box motif has also been identified, although its target gene is unknown.

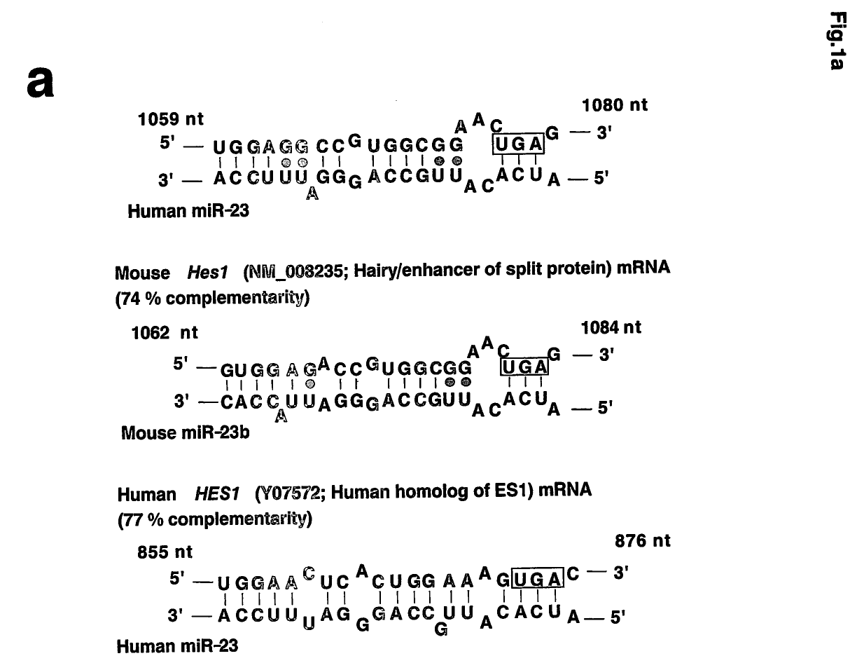

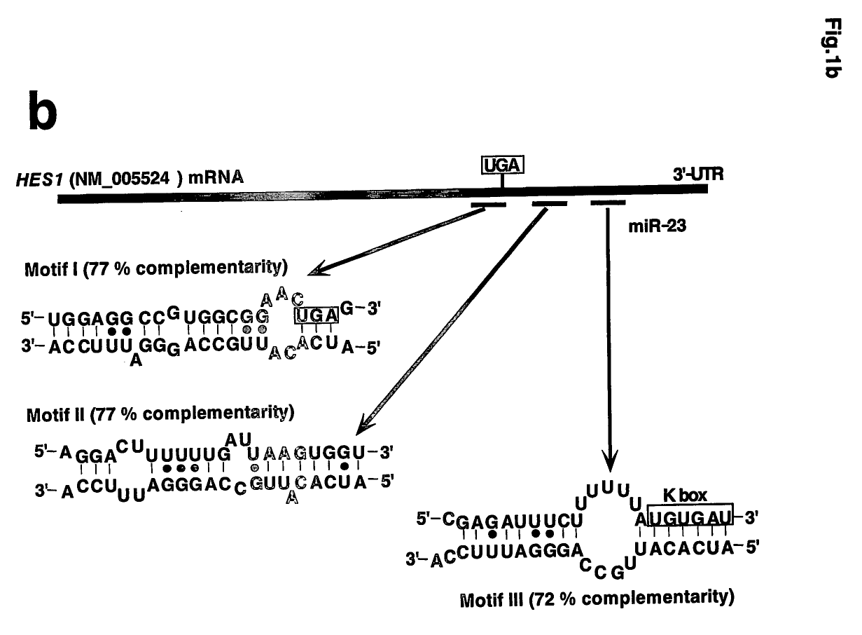

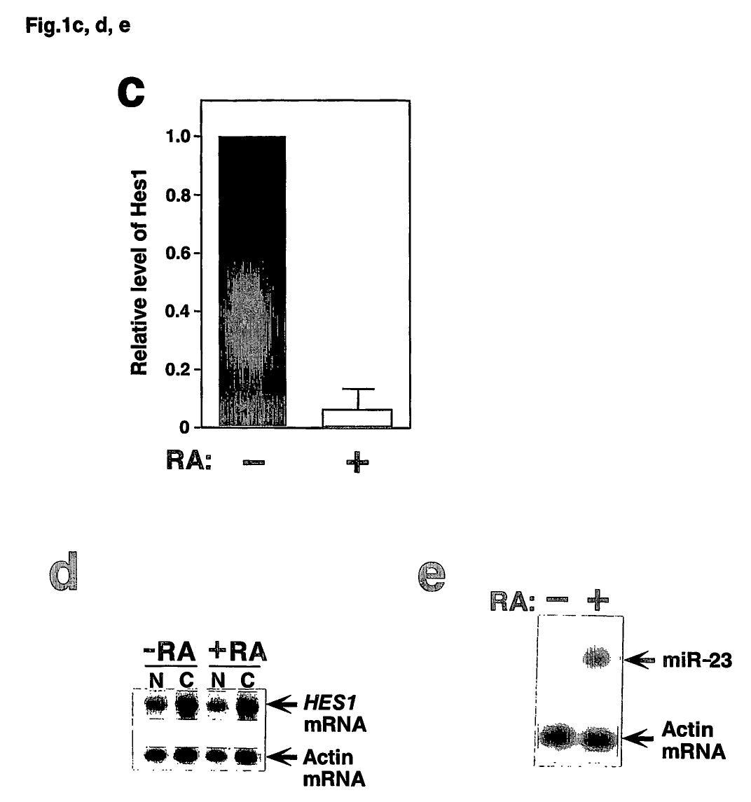

We initiated a study to investigate whether the human Hairy HES gene was the target of human miR-23. Hairy HES1 (Accession No. NM_{—}005524)³¹ is a basic helix-loop-helix (bHLM transcriptional repressor that is expressed in undifferentiated cells but not in differentiated cells³²⁻³⁴. It participates in the Notch signaling pathway in mammals and acts as an anti-differentiation factor. miR-23 aligned to a coding region of human HES1 (NM_{—}005524) mRNA near the termination codon and to mouse Hes1 mRNA (NM-008235) at nearly the same position as in human HES1 including the stop codon (FIG. 1a). A duplex of HES1 (NM_{—}005524) mRNA and miR-23 was also observed using a prediction program for mRNA secondary structure (Mulfold) suggesting that miR-23 may be conserved phylogenetically as a regulator of human and mouse Hes1. In addition, we also showed that miR-23 forms partial base-pairing with another mRNA similarly called HES1 Y07572 (human homolog of Escherichia coli and Zebrafish, Accession No. Y07572)₃₅ at nearly the same position as in human HES1 (NM_{—}005524) and mouse HES1 (NM_{—}008235) including the stop codon. A protein related to HES1 Y07572 with the same ElbB domain is involved in an early stage of the biosynthesis of isoprenoid compounds. Although Hairy HES1 (NM_{—}005524) has no similarity to Homolog HES1 Y07572) at the amino acid level, the target sequences for miR-23 in both genes have 70% similarity at the mRNA level). In addition, we found two other independent target sites of miR-23 in the 3′-untranslated region (UTR) of Hairy HES1 (NM_{—}005524) mRNA, which are designated motifs II and III (FIG. 1b). Moreover, we considered phylogenetic conservation between human and mouse Hairy Hes1 as a target of miR-23. The target of mouse Hairy Hes1 mRNA (nearly the same position as human Hes1 including the stop codon) exhibited significant complementarity (74%) to mouse miR-23b (FIG. 1a). A duplex of Hairy Hes1 and miR23 was observed using a prediction program of mRNA secondary structure (Mulfold). Thus, this observation suggests that the function of miR-23 is phylogenetically conserved as a regulator of human and mouse Hairy Hes1.

To confirm whether Hairy HES1 mRNA might be a target of miR-23, we used human NT2 cells, which are human embryonal carcinoma (EC) cells and differentiate into neural cells upon treatment with retinoic acid (RA)₃₆. We first examined the expression of Hairy HES1 during RA-induced differentiation by Western blotting and amplified ELISA assay. NT2 cells were treated with RA (5 μM) for 3 weeks. As shown in FIG. 1c, Hairy HES1 was easily detectable in undifferentiated NT2 cells. By contrast, Hairy HES1 was barely detectable in differentiated NT2 cells. However, as indicated by Northern blotting analysis, the level of Hairy HES1 mRNA in both nuclear (N) and cytoplasmic (C) fraction of cells remained unchanged during RA-induced differentiation (FIG. 1d). Moreover, in a sucrose sedimentation assay, an association between Hairy HES1 mRNA and polyribosomes was detected in both undifferentiated and differentiated NT2 cells (data not shown). Similar observations have been reported in the case of lin-4:lin-14 in C. elegans^{19,20}. These results suggest that the expression of Hairy HES1 might be regulated not at the cytoplasmic transport of Hairy HES1 mRNA but at the translation level during differentiation of NT2 cells. Next we examined the level of miR-23 during RA-induced differentiation by Northern blotting analysis. As shown in FIG. 1e, miR-23 was barely detectable in undifferentiated NT2 cells but was easily detected in differentiated NT2 cells. These results suggest that expression of miR-23 might be related to differentiation of NT2 cells.

Regulation of Expression of Hairy HES1 Gene by miR-23

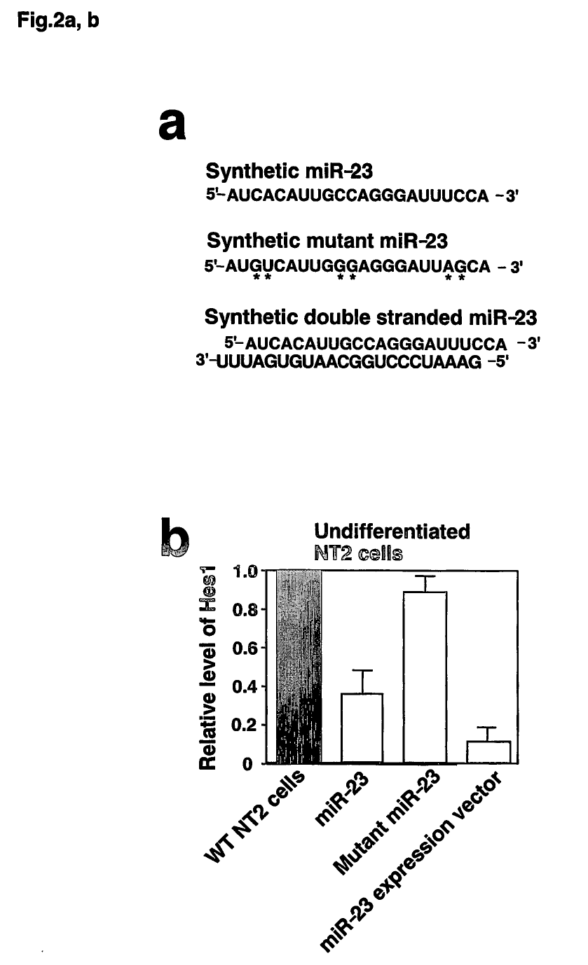

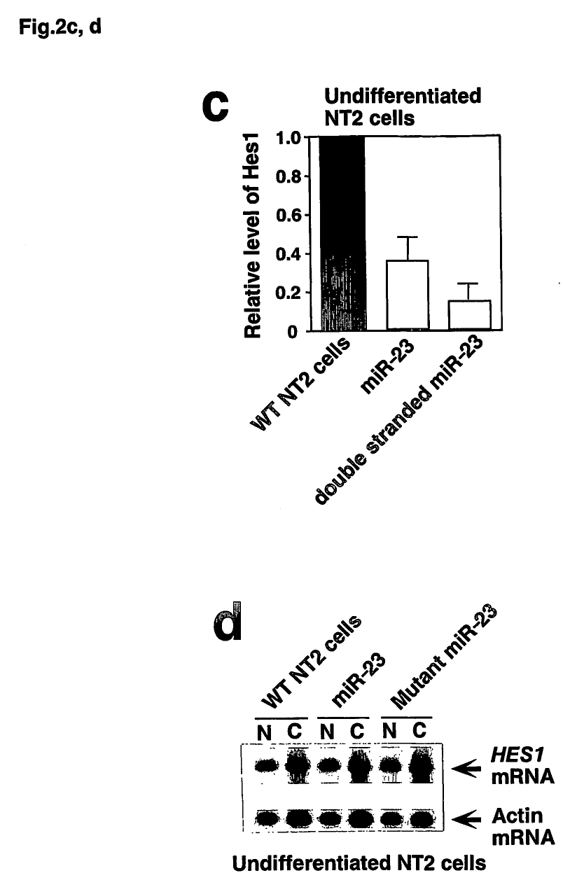

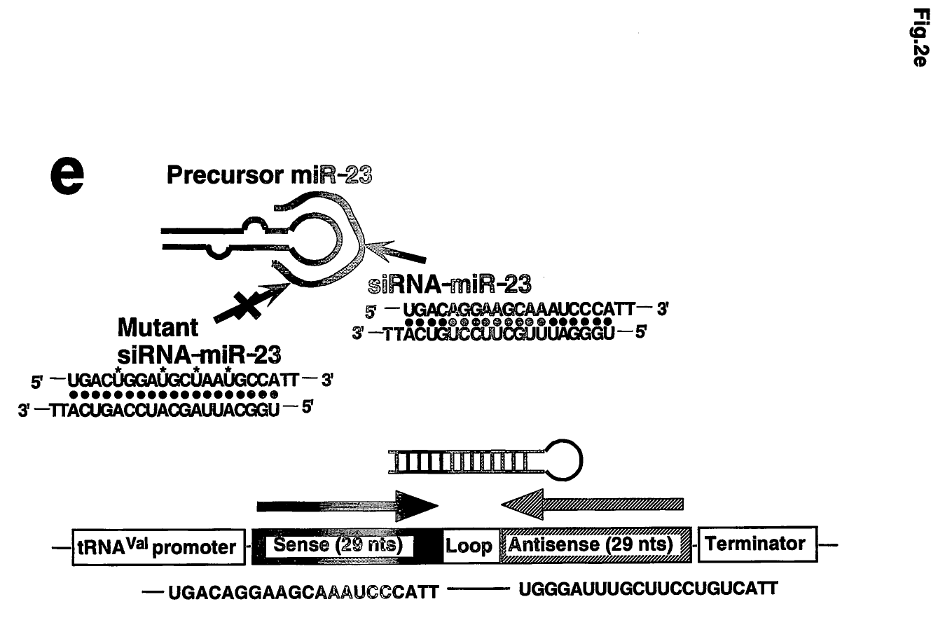

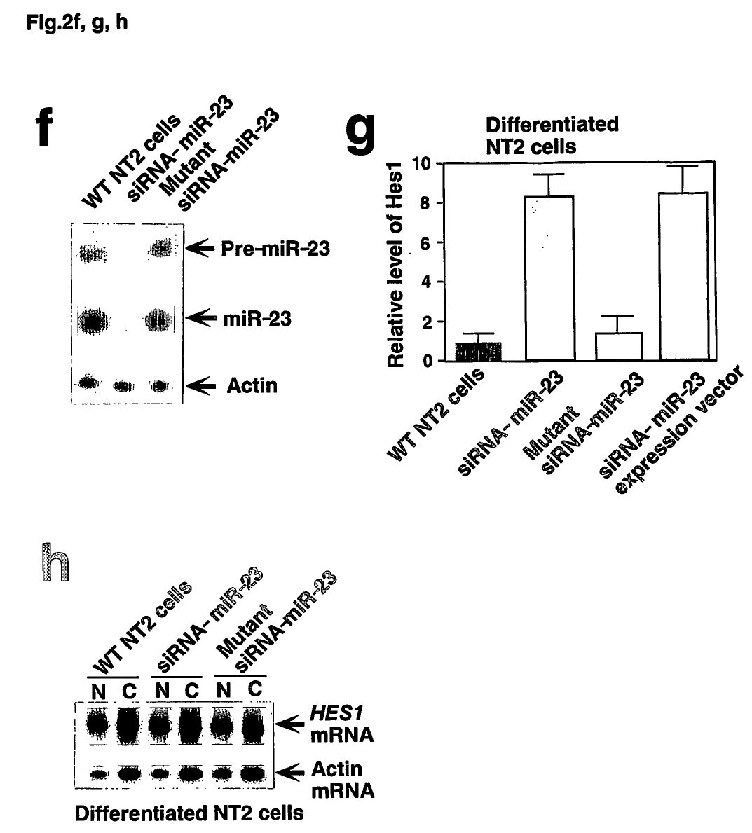

Next, to examine whether expression of the gene for Hes1 is regulated by miR-23, we introduced synthetic single stranded miR-23 or double stranded miR-23 (FIG. 2c) into undifferentiated NT2 cells. When synthetic miR-23 was introduced at 100 nM into undifferentiated NT2 cells, the intracellular level of Hes1 fell significantly (FIG. 2d). In addition, double stranded miR-23 has high efficiency compared with single stranded miR-23 in mammalian cells (FIG. 2c). By contrast, in the presence of synthetic mutant miR-23, the level of Hes1 in undifferentiated NT2 cells remained unchanged and similar to that in untreated wild-type (WT) NT2 cells (FIG. 2b). In addition, the level of Hes1 mRNA remained constant irrespective of the presence or absence of either synthetic miR-23 or mutant miR-23 (FIG. 2d). Similar results were obtained using pol III promoter (U6 and tRNA promoter)-dependent mRNA expression system (Fig. b). These results further suggest that synthetic miR-23 might suppress the expression of the gene for Hes1 at the translational level.

To examine the function of miR-23 in further detail, we tried to reduce the level of endogenous miR-23 using synthetic siRNA (siRNA-miR-23) targeted to a loop region of the precursor to miR-23 (FIG. 2e). siRNAs can induce the RNA interference-mediated (RNAi-mediated) sequence-specific silencing of gene expression in mammalian cells^{87,88}. RNAi refers to the sequence-specific silencing of gene expression that is induced by double-stranded RNAs (dsRNAs) in animals and plants^{89,40}. When 100 nM synthetic siRNA-miR-23 was introduced into differentiated NT2 cells, the intracellular level of precursor and mature miR-23 fell significantly (FIG. 20. By contrast, the level of Hes1 protein increased in the presence of siRNA-miR-23 (FIG. 2g). Importantly, mutant siRNA-miR-23 (FIG. 2e) did not affect expression of miR-23 or the level of Hes1 in NT2 cells. Moreover, the level of Hes1 mRNA was unaffected by synthetic siRNA-miR-23 (FIG. 2h). Similar results were obtained using pol m promoter (U6 and tRNA promoter)-dependent siRNA expression system that targeted to mRNAs (FIGS. 2f and 2g). These results indicate that the synthetic siRNA-miR-23 interfered with the function of miR-23 and, as a result, it promoted the accumulation of Hes1 protein. These results strengthen our hypothesis that miR23 regulates the expression of Hes1 at the post-transcriptional level.

Target Specificity of miR-23 in NT2 Cells

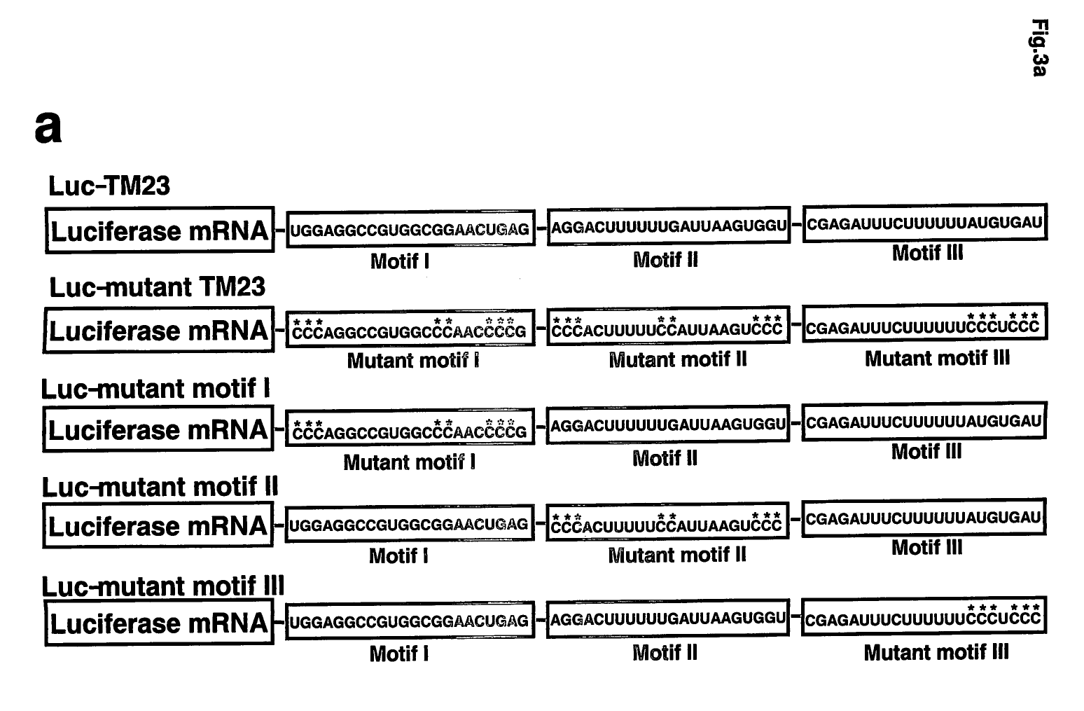

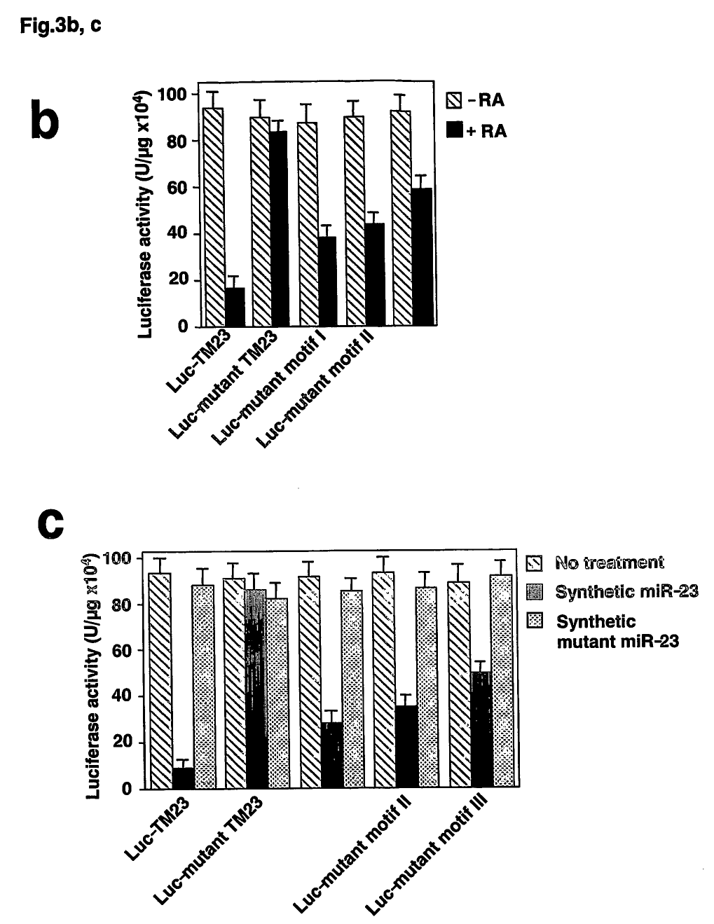

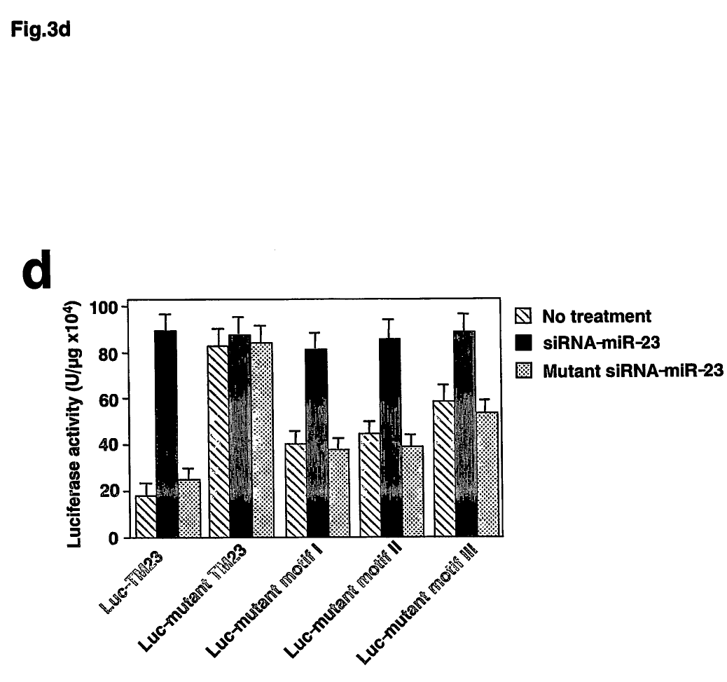

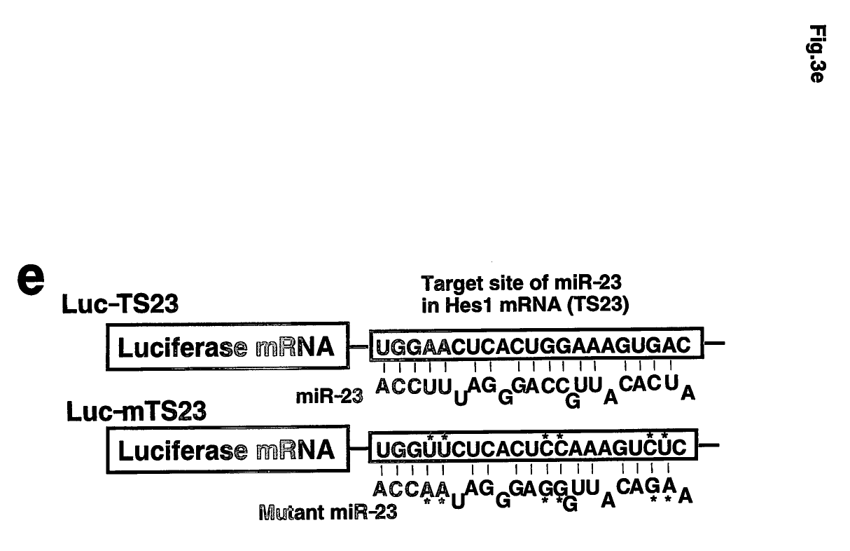

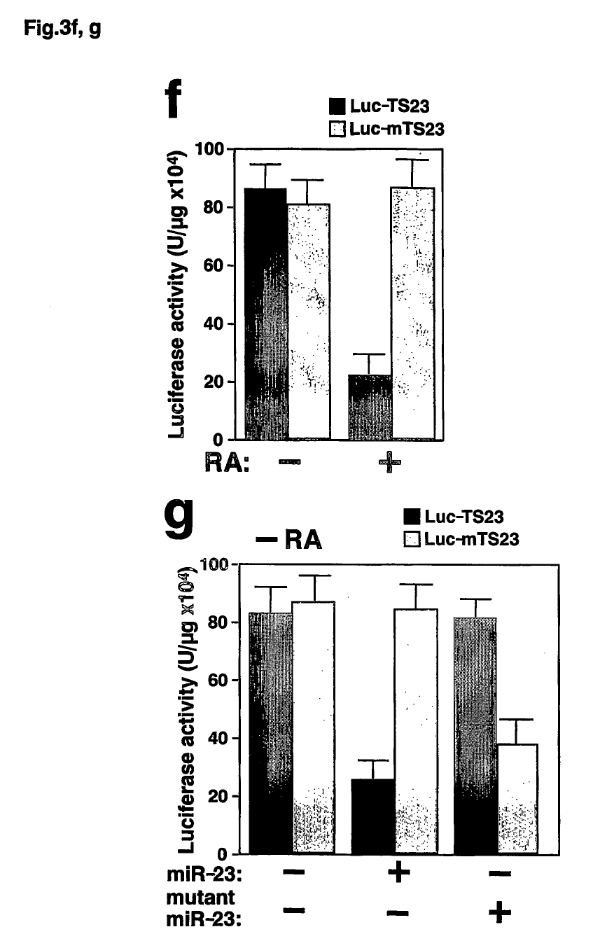

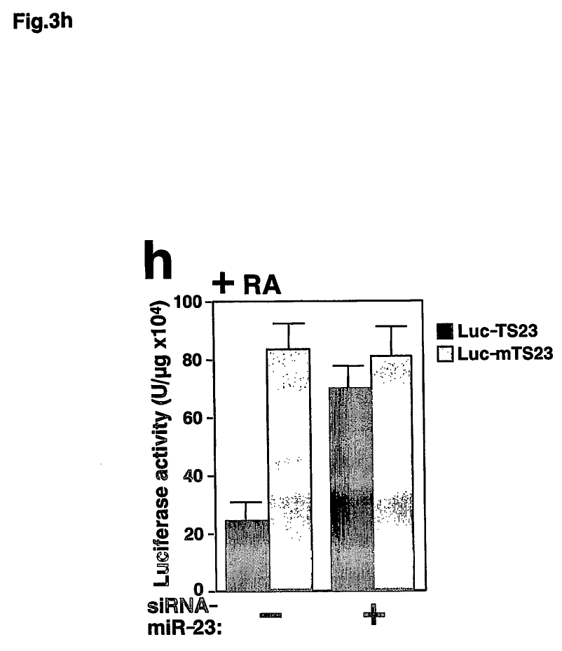

We thus examined whether the intact Hairy HES1 (NM-005524) 3′-UTR can confer regulation on a reporter gene in response to endogenous miR-23 in NT2 cells. To examine the target specificity of miR-23, we constructed plasmids for expression of a chimeric gene for luciferase that was fused 3′-UTR including the sequence of the three potential target motifs of miR-23 in Hairy HES1 mRNA (Luc-TM23; FIG. 3a). As a control, we designed the chimeric gene for luciferase that included the sequence of the mutated target motif(s) of miR-23 in Hairy HES1 mRNA (Luc-mutant TM23, Luc-mutant motif I, Luc-mutant motif II and Luc-mutant motif III; FIG. 3a). Then we transiently introduced each plasmid into NT2 cells. After incubation for 72 h, cells were harvested and lysed. Total proteins were used for the assays of luciferase activity using a luminometer (Lumat LB9501; Berthold, Bad Wildbad, Germany). As shown in FIG. 3b, we detected luciferase activity in undifferentiated NT2 cells (—RA) that expressed the gene for Luc-TM23. By contrast, the luciferase activity in differentiated cells (+RA) that expressed the gene for Luc-TM23 was significantly lower than that in undifferentiated cells (FIG. 3b). The luciferase activity in cells that expressed Luc-mutant TM23 did not changed during the differentiation. These results, under the conditions of natural endogenous level of miR-23, are in accord with the results with Hes 1. In addition, disruption of one target motif of miR-23 weakened the reduction level of the luciferase activity, suggesting the synergistic interaction of miR-23 with these three target sites.

Additionally, the luciferase activity of Luc-TM23 in undifferentiated NT2 cells that had been treated with synthetic miR-23 was significantly lower than that in untreated WT NT2 cells (rig. 3c). Mutant miR-23 did not affect the luciferase activity of the natural TM23-containing (the intact Hairy HES1 3′-UTR-containing) Luc-TM23 cells. By contrast, the luciferase activity in cells that expressed Luc-mutant TM23 remained the same in the presence or absence of synthetic miR-23 and in the presence of mutant miR-23. In addition, in the presence of synthetic miR-23, the luciferase activity in cells that expressed luciferase gene that has one mutant target motif of miR-23 was partially reduced.

Moreover, the luciferase activity in differentiated NT2 cells that had been treated with synthetic siRNA-miR-23 was higher than that in WT differentiated NT2 cells (FIG. 3d). These results suggest that the regulation of translation by the physiological level of miR-23 can be monitored by the attachment of its natural target UTR to the gene for luciferase and demonstrated that three independent target motifs in Hairy HES1 mRNA are certainly targets of miR-23.

Next, to examine the target specificity of miR-23, we constructed plasmids for expression of a chimeric gene for luciferase that was fused 3′-UTR sequence including the target sequence of miR-23 in Homolog HES1 mRNA (Luc-TS23; FIG. 3e). As a control we designed the chimeric gene for luciferase that was fused 3′-UTR sequence including the mutated target site of miR-23 in HES1 mRNA (Luc-mTS23; FIG. 3e). Then we introduced each plasmid into NT2 cells and obtained stable cell lines after selection with puromycin. As shown in FIG. 3b, we detected luciferase activity in undifferentiated NT2 cells that expressed the gene for Luc-TS23. By contrast, the luciferase activity in differentiated cells that expressed the gene for Luc-TS23 was lower than that in undifferentiated cells (FIG. 31). Additionally, the luciferase activity of Luc-TS23 in undifferentiated NT2 cells that had been treated with synthetic miR-23 was lower than that in untreated WT NT2 cells (FIG. 3g). Mutant miR-23 did not affect the luciferase activity of natural TS23-containing Luc-TS23 cells. To our surprise, a single-stranded mutant miR-23 RNA with appropriate compensatory mutations that restores its pairing to the mTS23 site (but retains the sites of mismatch, as shown at the mutant miR-23 region in FIG. 3h) rescued translational control of the mTS-luciferase (FIG. 3g), suggesting the possibility to create artificial microRNAs with slightly altered target sequences in the regulation of an arbitrarily chosen target gene. Moreover, the luciferase activity in differentiated NT2 cells that had been treated with synthetic siRNA-miR-23 was higher than that in WT differentiated NT2 cells (FIG. 3h). These results suggest that three TM23-I, -II and -III in Hairy HES1 (NM_{—}005524) mRNA and TS23 in Homolog HES1 (Y07572) mRNA are a target of miR-23 and that the natural near complementarity of TS23 to miR-23 is necessary for miR-23-mediated post-transcriptional silencing of gene expression.

The Role of miR-23 During RA-Induced Neuronal Differentiation

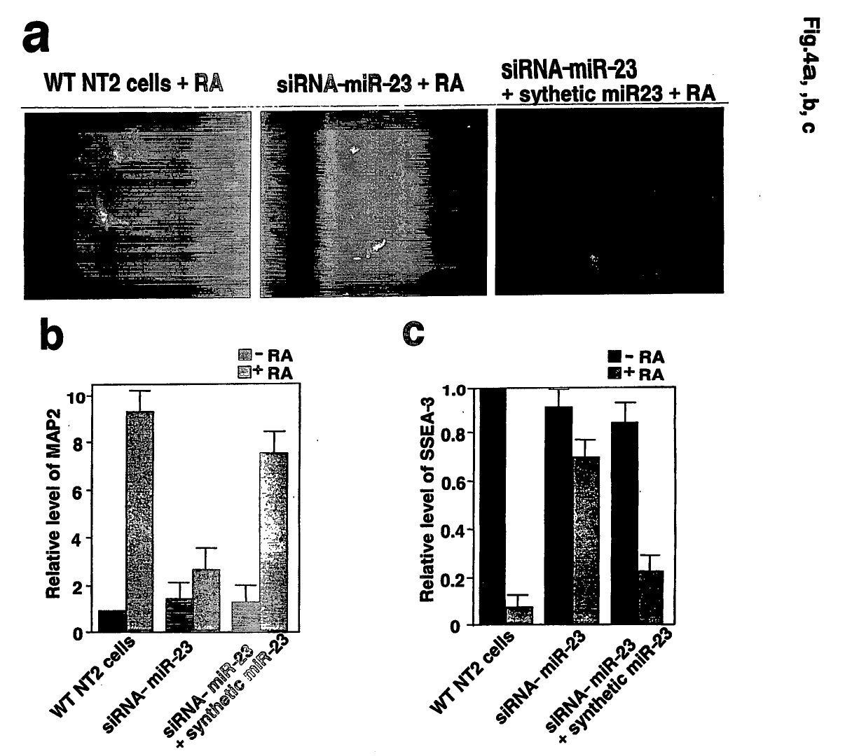

To examine the role of miR-23 during RA-induced neuronal differentiation of NT2 cells, we examined a phenotype of NT2 cells grown in the presence or absence of synthetic siRNA-miR-23 by immiuno-staining with SSEA-3- and MAP2-specific antibodies. SSEA-3 is expressed only in undifferentiated NT2 cells and MAP2 is expressed only in differentiated NT2 cells^{41,42}. Wild-type NT2 cells differentiate into neural cells upon treatment with RA (FIG. 4a; left panel). However, in the presence of siRNA-miR-23, NT2 cells did not differentiate into the neural cells after treatment with RA (FIG. 4a; middle panel). In addition, the level of MAP2, a differentiation marker, did not increase after the cells were treated with synthetic siRNA-miR-23, even though the level of MAP2 increased in WT differentiated NT2 cells (FIG. 4b). Accordingly, the level of SSEA-3, a marker of undifferentiated cells, did not decrease when NT2 cells were treated with synthetic siRNA-miR-23 and RA (FIG. 4c). However, the addition of synthetic miR-23 to cells that contained siRNA-miR-23 was able to reverse the effects of siRNA-miR-23, and these cells differentiated into neural cells upon treatment with RA (FIG. 4a; right panel), with an accompanying reduction in the level of SSEA-3 and induction of MAP2 expression. These results suggest that miR-23 plays a critical role during RA-induced neuronal differentiation.

Identification of Target Genes of Other mRNAs in Mammalian Cells

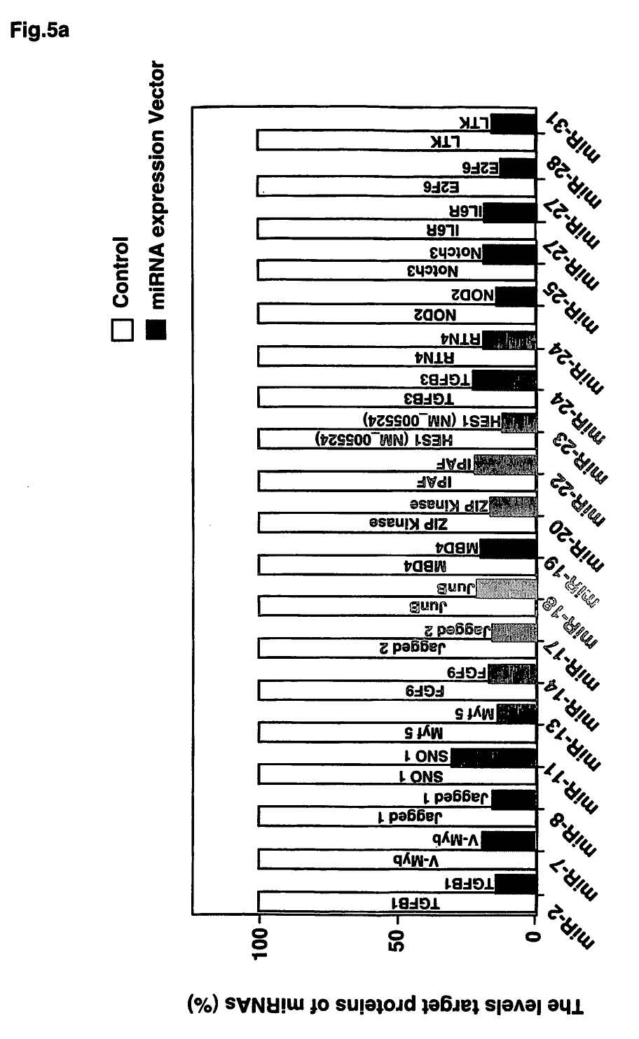

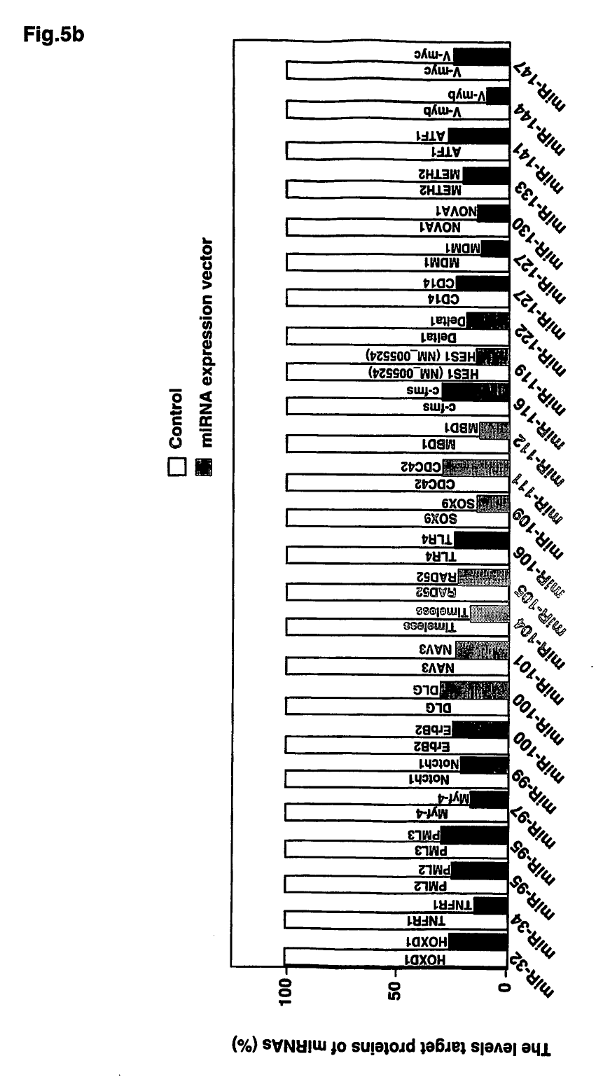

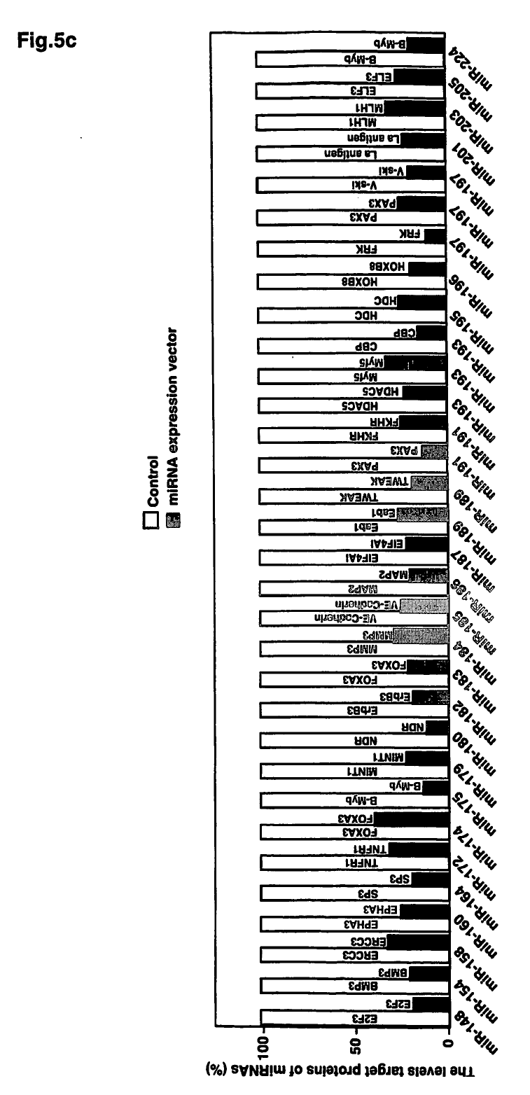

To understand function of mRNA in mammalian cell, we identified target genes of other mRNAs (more than 100 mRNAs) using BLAST search program (Table 1). Many differentiation (myeloid, myogenic, osteogenic and adipogenic)-associated factors are involved in these target genes. For example, expression of HOXB8 that is target of miR-196 is regulated in myeloid differentiation of HL60 cells. In addition, Myf-5 (target of miR-13) and Myf-4 (target of miR-97) are participate in myogenic differentiation. Moreover, TGF-beta (target of miR-13) and BMP3 (target of miR-154) associate with an osteogenic differentiation. Expression of these identified target genes were significantly reduced by synthetic and vector based mRNAs (FIG. 5). Since mRNA and siRNA (target to mRNAs) expression vector can regulate expression of above mRNAs, these expression vector have a high potential for regulation of differentiation and development of mammalian cells.

Culture and Transfection of Cells

Human NT2 cells were cultured in Dulbecco's modified Eagle's medium (DMEA) supplemented with 10% fetal bovine serum (FBS). Transfections were performed with the Effectin™ reagent (QIAGEN, Hilden, Germany) according to the manufacturer's protocol. Luc-TS23-expressing and Luc-mTS23-expressing NT2 cells were selected by incubation with puromycin for a week. Retinoic acid was used at 5 μM to be induced neuronal differentiation of NT2 cells for 3 weeks.

Preparation of mRNAs and siRNAs

Synthetic miR-23, mutant miR-23 and siRNAs directed against miR-23 were synthesized with a DNA/RNA synthesizer (model 394; PE Applied Biosystems, CA, USA). For generation of siRNAs, synthetic RNAs were annealed by a standard method³⁷. These siRNAs (100 nM) and synthetic miR-23 (2 μM were then introduced into NT2 cells using Oligofectamin™ (Invitrogen, Calif., USA) according to the manufacture's protocol.

Construction of Plasmids

For construction of the Luc-TS23 and Luc-mTS23 expression plasmids, we used the plasmid PRL-TK (Promega, Wis., USA). Five copies of the target site or of the mutant target site of miR-23 were inserted downstream of the gene for luciferase in pRL-TK. In the case of luciferase reporter genes bearing only one copy of the miR-23 target site, miR-23 barely affected translation of Luc-TS23, probably because of the strong SV40 promoter compared with the natural Hes1 promoter. The nucleotide sequence of each chimeric gene was confirmed by direct sequencing.

Preparation of the Nuclear Fraction and the Cytoplasmic Fraction of Cells

For the preparation of the cytoplasmic fraction, NT2 cells were washed twice with PBS and then resuspended in digitonin lysis buffer (50 mM HEPES/KOH, pH 7.5, 50 mM potassium acetate, 8 mM MgCl2, 2 mM EGTA and 50 μg/mL digitonin) on ice for 10 mins. The lysate was centrifuged at 1,000×g and the supernatant was collected as the cytoplasmic fraction. The pellets were resuspended in NP-40 lysis buffer (20 mM Tris-HCl, pH 7.5, 50 mM KCl, 10 mM NaCl, 1 mM EDTA and 0.5% NP-40) and held on ice for 10 mins and the resultant lysate was used as the nuclear fraction.

Northern Blotting Analysis

Cytoplasmic RNA and nuclear RNA were extracted and purified from the cytoplasmic fraction and the nuclear fraction, respectively, with ISOGEN™ reagent (Wako Co., Thyama, Japan). Thirty micrograms of total RNA per lane were loaded on a polyacrylamide gel (for detection of miR-23) or agarose gel (for detection of Hes1 mRNA). After electrophoresis, bands of RNA were transferred to a nylon membrane (Amersham Co., Buckinghamshire, UK). The synthetic DNA probe for Hes1 and synthetic RNA probe for miR-23 were labeled with ³²P by T4 polynucleotide kinase (Takara Shuzo Co., Kyoto, Japan). The level of actin was measured as an endogenous control.

Amplified Sandwich Enzyme-Linked Immunosorbent Assay (ELISA).

Amplified ELISA has been described elsewhere⁴⁵. NT2 cells, grown in the presence or absence of RA (5 μM, for 3 weeks), were harvested. Total protein was used in this assay. ELISA plates were coated with specific polyclonal antibodies against Hes1 (gift from Dr. Sudo at TORAY Co.), SSEA-3 (Santa Cruz) or MAP2 (UBI, VA, USA). After the plates had been washed three times, biotinylated second antibodies, followed by horseradish peroxidase-conjugated (HRP-conjugated) streptavidin, were added at room temperature. Absorbance was monitored at 490 nm with a microplate reader after addition of phenylenediamine (Sigma-Aldrich Co., MO, USA).

Western Blotting Analysis

Total proteins (each 20 μg) were resolved by SDS-PAGE (10% polyacrylamide gel) and transferred to a polyvinylene difluoride (PVDF) membrane (Funakoshi Co., Tokyo, Japan) by electroblotting. Immune complexes were visualized with ECL kit (Amersham Co., Buckinghamshire, UK) using specific polyclonal antibodies against HES1 (gift from Dr. Sudo at TORAY Co.). The relative levels of HES1 was normalized with the level of actin.

Assay of Luciferase Activity.

miR-23-siRNAs, synthetic miR-23 and mutant miR-23 were introduced into NT2 cells that expressed Luc-TS23 or Luc-mTS23 using Oligofectamin™ (Invitrogen) according to the manufacture protocol. After incubation for 72 h, cells were harvested and lysed. Total protein was assayed for luciferase activity using a luminometer (Lumat LB9501; Berthold, Bad Wildbad, Germany).

Immunostaining

Cells were fixed in paraformaldehyde in PBS for 1 h. Then cells were incubated with polyclonal antibody against a SBEA-3 (Santa Cruz) or against MAP2 (UBI) for 2 h. Fluoresceinisothiocyanate-conjugated (FITC-conjugated) or rhodamine-conjugated secondary antibodies were then added. Nuclei of NT2 cells were stained with 4-diamidino-2-phenylindole (DAPI; Sigma-Aldrich Co.).

### REFERENCES

All references (e.g., books, articles, applications, and patents) cited in this specification are indicative of the level of skill in the art and their disclosures are incorporated herein in their entirety.
1. Eddy, S. R. Non-coding RNA genes and the modern RNA world. Nature Reviews Genetics 2, 919-929 (2001).
2. Lee, R. C., Feinbaum, R. L., & Ambros, V. The C. elegans heterochronic gene lin-4 encodes small RNAs with antisense complementarity to lin-14. Cell 75, 843-854 (1993).
3. Reinhart, B. J., Slack, F. J., Basson, M., Pasquinelli, A. E., Bettinger, J. C., Rougvie, A. E., Horvitz, H. R., & Ruvkun, G. The 21-nucleotide let-7 RNA regulates developmental timing in C. elegans. Nature 403, 901-906 (2000).
4. Pasquinelli, A. E., Reinhart, B. J., Slack, F., Martindale, M. Q., Kuroda, M. I., Maller, B., Hayward, D. C., Ball, E. E., Degnan, B., Muller, P., Spring, J., Srinivasan, A., Fishman, M., Finnerty, J., Corbo, J., Levine, M., Leahy, P., Davidson, E., & Ruvkun, G. Conservation of the sequence and temporal expression of let-7 heterochronic regulatory RNA. Nature 408, 86-89 (2000).
5. Slack, F. J., Basson, M., Liu, Z., Ambros, V., Horvitz, H. R., & Ruvkun, G. The lin-41 RBCC gene acts in the C. elegans heterochronic pathway between the let-7 regulatory RNA end the LIN-29 transcription factor. Mol. Cell 5, 659-669 (2000).
6. Lau, N. C., Lim, L. P., Weinstein, E. G., & Bartel, D. P. An abundant class of tiny RNAs with probable regulatory roles in C. elegans. Science, 294, 858-862 (2001).
7. Lee, R. C., & Ambros, V. An extensive class of small RNAs in Caenorhabditis elegans. Science, 294, 862-864 (2001).
8. Lagos-Quintana, M, Rauhut, R, Yalcin, A., Meyer, J., Lendeckel, W., & Tuschl, T. Identification of novel genes coding for small expressed RNAs. Science 294, 853-858 (2001).
9. Mourelatos, Z., Dostie, J., Paushiin, S., Sharma, A., Charroux, B., Abel, L., Rappsilber, J., Mann, M., & Dreyfuss, G. miRNPs: a novel class of ribonucleoproteins containing numerous microRNAs. Genes Dev. 16, 720-728 (2002).
10. Reinhart, B. J., Weinstein, E. G., Rhoades, M. W., Bartel, B., & Bartel, D. P. MicroRNAs in plants. Genes Dev. 16, 1616-1626 (2002).
11. Lagos-Quintana, M., Rauhut, R., Yalcin, A., Meyer, J., Lendeckel, W., & Tuschl, T. Identification of tissue-specific microRNAs from mouse. Curr. Biol. 12, 735-739 (2002).
12. Rhoades, M. W., Reinhart, B. J., Lim, L. P., Burge, C. B., Bartel, B., & Bartel, D. P. Prediction of plant microRNA targets. Cell 110, 513-520 (2002).
13. Lagos-Quintana, M., Rauhut, R., Meyer, J., Borkhardt, A., & Tuschl, T. New microRNAs from mouse and human. RNA 9, 175-179 (2003).
14. Lim, L. P., Glasner, M. E., Yekta, S., Burge, C. B., & Bartel, D. P. Vertebrate microRNA genes. Science 299, 1540 (2003).
15. Lim, L. P., Lau, N. C., Weinstein, E. G., Abdelhakim, A., Yekta, S., Rhoades, M. W., Burge, C. B., & Bartel, D. P. The microRNAs of Caenorhabditis elegans. Genes Dev 17, 991-1008 (2003).
16. Dostie, J., Mourelatos, Z., Yang, M., Sharma, A., & Dreyfuss, G. Numerous microRNPs in neuronal cells containing novel microRNAs. RNA 9, 180-186 (2003).
17. Llave, C., Xie, Z., Kasschau, K. D., & Carrington, J. C. Cleavage of Scarecrow-like mRNA targets directed by a class of Arabidopsis mRNA. Science 297, 2053-2056 (2002).
18. Tang, G., Reinhart, B. J., Bartel, D. P., & Zamore, P. D. A biochemical framework for RNA silencing in plants. Genes Dev. 17, 49-63 (2003).
19. Olsen, P. H., & Ambros, V. The lin-4 regulatory RNA controls developmental timing in Caenorhabditis elegans by blocking LIN-14 protein synthesis after the initiation of translation. Dev. Biol. 216, 671-680 (1999).
20. Seggerson, K., Tang, L., & Moss, E. G. Two genetic circuits repress the Caenorhabditis elegans heterochronic gene lin-28 after translation initiation. Dev Biol. 243, 215-225 (2002).
21. Lee, Y, Jeon, K, Lee, J. T., Kim, S., & Kim, V. N. MicroRNA maturation: stepwise processing and subcellular localization. EMBO J. 21, 4663-4670 (2002).
22. Grishok, A., Pasquinelli, A. E., Conte, D., Li, N., Parrish, S., Ha, I., Baillie, D. L., Fire, A., Ruvkun, G., & Mello, C. C. Genes and mechanisms related to RNA interference regulate expression of the small temporal RNAs that control C. elegans developmental timing. Cell 106, 23-34 (2001).
23. Hutvagner, G., McLachlan, J., Pasquineri, A. E., Balint, E., Tuschl, T., & Zamore, P. D. A cellular function for the RNA-interference enzyme Dicer in the maturation of the let-7 small temporal RNA. Science 293, 834-838 (2001).
24. Ketting, R. F., Fischer, S. E., Bernstein, E., Sijen, T, Hannon, G. J., & Plasterk, R. H. Dicer functions in RNA interference and in synthesis of small RNA involved in developmental timing in C. elegans. Genes Dev. 15, 2654-2659 (2001).
25. Martinez, J., Patkaniowska, A., Urlaub, H., Luhrmann, R., & Tuschl, T. Single-stranded antisense siRNAs guide target RNA cleavage in RNAi. Cell 110, 563-574 (2002).
26. Zeng, Y, Wagner, E. J., & Cullen, B. R. Both natural and designed micro RNAs can inhibit the expression of cognate mRNAs when expressed in human cells. Mol. Cell. 9, 1327-1333 (2002).
27. Zeng, Y., & Cullen, B. R. Sequence requirements for microRNA processing and function in human cells. RNA 9, 112-123 (2003).
28. Lai, E. C., and Posakony, J. W. (1997) The Bearded box, a novel 3′ UTR sequence motif, mediates negative post-transcriptional regulation of Bearded and Enhancer of split Complex gene expression Development 124: 4847-4856.
29. Lai, E. C., Burks, C., and Posakony, J. W. (1998) The K box, a conserved 3′ UTR sequence motif, negatively regulates accumulation of enhancer of split complex transcripts. Development 125: 4077-4088.
30. Lai, E. C. (2002) Micro RNAs are complementary to 3′ UTR sequence motifs that mediate negative post-transcriptional regulation. Nature Genet. 30: 363-364.
31. Feder, J. N., Li, L., Jan, L. Y., and Jan, Y. N. (1994) Genomic cloning and chromosomal localization of HRY, the human homolog to the Drosophila segmentation gene, hairy. Genomics, 20: 56-61.
32. Ohtsuka, T., Ishibashi, M., Gradwohl, G., Nakanishi, S., Guillemot, F., & Kageyama, R. Hes1 and Hes5 as notch effectors in mammalian neuronal differentiation. EMBO J. 18, 2196-2207 (1999).
33. Kageyama, R., Ohtsuka, T., & Tomita, K The bHLH gene Hes1 regulates differentiation of multiple cell types. Mol. Cells 29, 1-7 (2000).
34. Ohtsuka, T., Sakamoto, M., Guillemot, F., & Kageyama, R. Roles of the basic helix-loop-helix genes Hes1 and Hes5 in expansion of neural stem cells of the developing brain. J. Biol. Chem. 276, 30467-30474 (2001).
35. Scott, H. S., Chen, H., Rossier, C., Lalioti, M. D., and Antonarakis, S. E. (1997) Isolation of a human gene MES1) with homology to an Escherichia coli and a zebrafish protein that maps to chromosome 21q22.3. Human Genet. 99: 616-623.
36. Cheung, W. M., Chu, P. W., Lung, C. H., & Ip, N.Y. Expression of retinoid receptors during the retinoic acid-induced neuronal differentiation of human embryonal carcinoma cells. J. Neurochem. 75, 34.40 (2000).
37. Elbashir, S. M., Harborth, J., Lendeckel, W., Yalcin, A., Weber, K, & Thschl, T. Duplexes of 21-nucleotide RNAs mediate RNA interference in mammalian cell culture. Nature 411, 494-498 (2001).
38. McManus, M. T. & Sharp P. A. Gene silencing in mammals by small interfering RNAs. Nature Reviews Genetics 3, 737-747 (2002).
39. Fire A, Xu S., Montgomery, M. K, Kostas, S. A., Driver, S. E., & Mello, C. C. Potent and specific genetic interference by double-stranded RNA in Caenorhabditis elegans. Nature 391, 806-811 (1998).
40. Hammond, S. M., Caudy, A. A., & Hannon, G. J. Post-transcriptional gene silencing by double-stranded RNA. Nature Reviews Genetics 2, 110-119 (2001).
41. Pleasure, S. J., & Lee, V. M. NTera 2 cells: a human cell line which displays characteristics expected of a human committed neuronal progenitor cell. J. Neuzosci. Res. 35, 585-602 (1993).
42. Kurie, J. M., Brown, P., Salk, E., Scheinberg, D., Birrer, M., Deutsch, P., & Dmitrovsky, E. Cooperation between retinoic acid and phorbol esters enhances human teratocarcinoma differentiation. Differentiation 54, 115-122 (1993).
43. Hutvagner, G., & Zamore, P. D. A microRNA in a multiple-turnover RNAi enzyme complex. Science 297, 2056-2060 (2002).
44. Brennecke, J., Hipfuer, D. R., Stark, A., Russell, R. B., & Cohen, S. M. bantam Encodes a Developmentally Regulated microRNA that Controls Cell Proliferation and Regulates the Proapoptotic Gene hid in Drosophila. Cell 113, 25-36 (2003).
45. Kawasaki, H., Eckner, R., Yao, T. P., Taira, K., Chiu, R., Livingston, D. M., & Yokoyama, K. K. Distinct roles of the co-activators p300 and CBP in retinoic-acid-induced F9-cell differentiation. Nature 393, 284-289 (1998).

### OTHER EMBODIMENTS

It is to be understood that while the invention has been described in conjunction with the detailed description thereof the foregoing description is intended to illustrate and not limit the scope of the invention, which is defined by the scope of the appended claims. Other aspects, advantages, and modifications are within the scope of the following claims.

TABLE 1

| miRNA | miRNA Target | Accession No. | miRNA Target | Accession No. | miRNA Target | Accession No. |
|---|---|---|---|---|---|---|
| miR-1 | dbl | X12556 | | | | |
| miR-2-1 | TGFBI | NM_000660 | | | | |
| miR-5 | TGF alpha | NM_003236 | | | | |
| miR-7 | V-Myb | NM_005375 | a-myb | X66087 | | |
| miR-8 | c-cbl | X57110 | Jagged1 | NM_000214 | | |
| miR-11 | SNO I (N, A) | Z19588 | | | | |
| miR-12 | Activin beta | AF412024 | | | | |
| miR-13 | Myf-5 | NM_005593 | | | | |
| miR-14 | FGF9 | NM_002010 | SHOTb | AJ002368 | SHOTa | AJ002367 |
| miR-15 | RON | X70040 | | | | |
| miR-16 | SMURF1 | NM_020429 | | | | |
| miR-17 | jagged 2 | NM_002226 | DAPK3 | NM_001348 | | |
| miR-18 | JunB | X51345 | RAD51 | NM_002875 | ING1 | NM_198219 |
| miR-19 | MBD4 | NM_003925 | MED1 | AF114784 | | |
| miR-20 | ZIP Kinase | AB022341 | | | | |
| miR-21 | Endomucin | NM_016242 | HUS1 | NM_004507 | V-ETS | NM_004449 |
| miR-22 | IPAF | AY035391 | | | | |
| miR-23 | HES1 | NM_005524 | HES1 | Y07572 | | |
| miR-24 | TGF-B3 | NM_003239 | CALDESMON | NM_033138 | RTN4 | NM_020532 |
| miR-25 | enaptin | AF535142 | VENTX2 | NM_014468 | NOD2 | AF178930.1 |
| miR-26 | AMPD3 | M84721 | EGR2 | J04076 | | |
| miR-27 | IL1A | AF536338 | NOTCH3 | U97669 | IL6R | NM_000565 |
| miR-28 | E2F6 | NM_001952 | | | | |
| miR-29 | laminin alpha | NM_005559 | | | | |
| miR-30 | DNA Pol alpha | NM_002689 | | | | |
| miR-31 | LTK | NM_002344 | | | | |
| miR-32 | HOXD1 | NM_024501 | | | | |
| miR-33 | laminin gamma | NM_002293 | | | | |
| miR-34 | TNFR1 | BC010140 | | | | |
| miR-92 | Villin2 | NM_003379 | Lin28 | NM_024674 | | |
| miR-93 | Frizzled homolog 5 | NM_003468 | | | | |
| miR-94 | ACF1 | AF213467 | | | | |
| miR-95 | MSX2 | X69295 | PML3 | M79464 | PML2 | M79463 |
| miR-96 | ADFP | NM_001122 | | | | |
| miR-97 | Myf-4 | NM_002479 | | | | |
| miR-98 | Sox-5 | NM_006940 | | | | |
| miR-99 | Notch1 | NM_017617 | MYCBP | NM_012333 | | |
| miR-100 | ErbB2 | M11730 | Tob | NM_005749 | DLG1 | NM_004087 |
| miR-101 | DNA Pol theta | NM_006596 | NAV3 | NM_014903 | YAP1 | NM_006106 |
| miR-103 | CREB3 | NM_006368 | | | | |
| miR-104 | Timeless | BC050557 | MAST1 | AF347693 | | |
| miR-105 | RAD52 | NM_002879 | | | | |
| miR-106 | TLR4 | NM_138554 | HECA | NM_016217 | | |
| miR-107 | CREB3 | NM_006368 | MAP6 | XM_166256 | | |
| miR-109 | SOX9 | NM_000346 | | | | |
| miR-110 | HOXA5 | NM_019102 | | | | |
| miR-111 | CDC42 | BC018266 | | | | |
| miR-112 | Desmuslin | NM_145728 | MBD1 | NM_015846 | | |
| miR-113 | TFIIIC Box B-binding subunit | U02619 | | | | |
| miR-114 | profilin 2 | NM_053024.1 | | | | |
| miR-116 | c-fms | X03663 | | | | |
| miR-119 | HES1 | NM_005524 | | | | |
| miR-122 | Delta1 (DLL1) | NM_005618 | | | | |
| miR-125 | FACL5 | NM_016234 | | | | |
| miR-126 | DLGAP2 | NM_004745 | | | | |
| miR-127 | TFIIIC Box B-binding subunit | U02619 | EPHA5 | NM_004439 | CD14 | NM_000591 |
| miR-129 | TFIIH | AB088103.1 | RPC32 | NM_006467 | | |
| miR-130 | RPC2 | NM_018082 | NOVA1 | NM_002515 | | |
| miR-132 | RecQ5 | NM_004259 | | | | |
| miR-133 | METH2 | AF060153 | | | | |
| miR-134 | MOST2 | NM_020250 | | | | |
| miR-136 | SOX7 | NM_031439 | | | | |
| miR-138 | Intergin B1 | X07979 | | | | |
| miR-140 | Desmin | NM_001927 | | | | |
| miR-141 | POT1 | NM_015450 | ATF1 | NM_005171 | | |
| miR-144 | V-myb | NM_005375 | | | | |
| miR-145 | HLX1 | NM_021958 | | | | |
| miR-146 | GABA Transport protein | U76343 | IRBP | M22453 | | |
| miR-147 | V-myc | NM_005378 | | | | |
| miR-148 | BAG5 | AF095195 | E2F3 | NM_001949 | | |
| miR-149 | PLAB | U88323 | | | | |
| miR-150 | BAF600 | AF348492.1 | MEST | NM_002402 | | |
| miR-151 | ALK-4 | Z22536 | | | | |
| miR-153 | TLL2 | NM_012465 | | | | |
| miR-154 | RIGB | AF525085 | BMP3 | NM_001201 | | |
| miR-157 | POT1 (J) | NM_015450 | | | | |
| miR-158 | ERCC3 (J) | M31899 | | | | |
| miR-160 | TBX22 (J) | NM_016954 | EPHA3 | NM_005233 | | |
| miR-162 | BAP1 (J) | AF045581 | | | | |
| miR-164 | SP3 (J) | NM_003111 | | | | |
| miR-172 | TNFR1 | BC010140 | MBD5 | NM_018328 | | |
| miR-173 | TEF1 (D) | X84839 | | | | |
| miR-174 | FOXA3 | NM_004497 | FGF12 | NM_021032 | | |
| miR-175 | ELF2 | AF256222 | B-myb | X13293 | | |
| miR-176 | MAP1A | NM_002373 | | | | |
| miR-177 | Myosin 5B | AY274809 | | | | |
| miR-178 | NEDL1 (D | AB048365 | | | | |
| miR-179 | MINT1 (D) | AF029106 | RNA helicase A (D) | L13848 | | |
| miR-180 | PARX (D) | AF439781 | MMP26 (D) | NM_021801 | NDR (D) | AY255672 |
| miR-182 | ERBB3 (T) | M29366 | | | | |
| miR-183 | FOXA3 | NM_004497 | | | | |
| miR-184 | MMP3 (D) | AF405705 | Crossveinless-2 (D) | AY324650 | | |
| miR-185 | VE-CADHERIN | X79981 | CADHERIN5 | NM_001795 | | |
| miR-186 | MAP2 (D) | NM_002374 | | | | |
| miR-187 | TAFII55 | NM_005642 | EIF4AI | NM_001416 | | |
| miR-188 | EFG2 | NM_032380 | | | | |
| miR-189 | Eab1 (D) | U71207 | TWEAK (D) | AF030099 | | |
| miR-191 | PAX3 | NM_181457 | FKHR | UO2310 | | |
| miR-192 | Synaptotagmin1 (D) 3UTR | U19921 | | | | |
| miR-193 | HDAC5 | NM_005474 | Myf5 | NM_005593 | CBP | NM_004380 |
| miR-195 | hHDC | AB033492 | | | | |
| miR-196 | HOXB8 | NM_024016 | HOXB7 Promoter | AJ414528 | | |
| miR-197 | FRK | NM_002031 | PAX3 5-UTR | AJ007392 | V-ski | NM_003036 |
| miR-199 | FAST1 | AF076292 | | | | |
| miR-201 | La antigen | X97869.1 | | | | |
| miR-203 | MLH1 | NM_000249 | | | | |
| miR-205 | ELF3 | AF517841 | | | | |
| miR-224 | B-Myb | X13293 | | | | |

| miRNA | miRNA Target | Accession No. | miRNA Target | Accession No. | miRNA Target | Accession No. |
|---|---|---|---|---|---|---|
| miR-1 | | | | | | |
| miR-2-1 | | | | | | |
| miR-5 | | | | | | |
| miR-7 | | | | | | |
| miR-8 | | | | | | |
| miR-11 | | | | | | |
| miR-12 | | | | | | |
| miR-13 | | | | | | |
| miR-14 | | | | | | |
| miR-15 | | | | | | |
| miR-16 | | | | | | |
| miR-17 | | | | | | |
| miR-18 | | | | | | |
| miR-19 | | | | | | |
| miR-20 | | | | | | |
| miR-21 | SDK1 | NM_152744 | | | | |
| miR-22 | | | | | | |
| miR-23 | | | | | | |
| miR-24 | | | | | | |
| miR-25 | | | | | | |
| miR-26 | | | | | | |
| miR-27 | BMPR2 | NM_001204 | | | | |
| miR-28 | | | | | | |
| miR-29 | | | | | | |
| miR-30 | | | | | | |
| miR-31 | | | | | | |
| miR-32 | | | | | | |
| miR-33 | | | | | | |
| miR-34 | | | | | | |
| miR-92 | | | | | | |
| miR-93 | | | | | | |
| miR-94 | | | | | | |
| miR-95 | | | | | | |
| miR-96 | | | | | | |
| miR-97 | | | | | | |
| miR-98 | | | | | | |
| miR-99 | | | | | | |
| miR-100 | PDCD10 | NM_007217 | BNIP2 | NM_004330 | | |
| miR-101 | CDK7 | NM_001239 | Importin beta2 | BC040340 | V-ros | NM_002944 |
| miR-103 | | | | | | |
| miR-104 | | | | | | |
| miR-105 | | | | | | |
| miR-106 | | | | | | |
| miR-107 | | | | | | |
| miR-109 | | | | | | |
| miR-110 | | | | | | |
| miR-111 | | | | | | |
| miR-112 | | | | | | |
| miR-113 | | | | | | |
| miR-114 | | | | | | |
| miR-116 | | | | | | |
| miR-119 | | | | | | |
| miR-122 | | | | | | |
| miR-125 | | | | | | |
| miR-126 | | | | | | |
| miR-127 | mdm1 | NM_017440 | | | | |
| miR-129 | | | | | | |
| miR-130 | | | | | | |
| miR-132 | | | | | | |
| miR-133 | | | | | | |
| miR-134 | | | | | | |
| miR-136 | | | | | | |
| miR-138 | | | | | | |
| miR-140 | | | | | | |
| miR-141 | | | | | | |
| miR-144 | | | | | | |
| miR-145 | | | | | | |
| miR-146 | | | | | | |
| miR-147 | | | | | | |
| miR-148 | | | | | | |
| miR-149 | | | | | | |
| miR-150 | | | | | | |
| miR-151 | | | | | | |
| miR-153 | | | | | | |
| miR-154 | | | | | | |
| miR-157 | | | | | | |
| miR-158 | | | | | | |
| miR-160 | | | | | | |
| miR-162 | | | | | | |
| miR-164 | | | | | | |
| miR-172 | | | | | | |
| miR-173 | | | | | | |
| miR-174 | | | | | | |
| miR-175 | | | | | | |
| miR-176 | | | | | | |
| miR-177 | | | | | | |
| miR-178 | | | | | | |
| miR-179 | | | | | | |
| miR-180 | | | | | | |
| miR-182 | | | | | | |
| miR-183 | | | | | | |
| miR-184 | | | | | | |
| miR-185 | | | | | | |
| miR-186 | | | | | | |
| miR-187 | | | | | | |
| miR-188 | | | | | | |
| miR-189 | | | | | | |
| miR-191 | | | | | | |
| miR-192 | | | | | | |
| miR-193 | | | | | | |
| miR-195 | | | | | | |
| miR-196 | | | | | | |
| miR-197 | | | | | | |
| miR-199 | | | | | | |
| miR-201 | | | | | | |
| miR-203 | | | | | | |
| miR-205 | | | | | | |
| miR-224 | | | | | | |

1. A method for modulating expression of a target gene in a cell, the method comprising introducing into the cell a polynucleotide that forms a duplex region with an mRNA transcribed from said target gene, wherein said duplex region comprises a mammalian mRNA target region.

2. The method of claim 1, wherein the miRNA target region comprises a sequence having at least about 70% identity to a polynucleotide selected from SEQ ID Nos: 5-11, 13, and 121-290.

3. The method of claim 1, wherein the polynucleotide is an miRNA or a precursor thereof, or a vector encoding said miRNA or a precursor thereof.

4. The method of claim 3, wherein the miRNA comprises a sequence having at least about 70% identity to a polynucleotide selected from SEQ ID Nos: 1, 3, 12, and 14-120.

5. The method of claim 4, wherein the miRNA or precursor thereof is selected from the group consisting of: miR-1, miR-2-1, miR-5, miR-7, miR-8, miR-11, miR-12, miR-13, miR-14, miR-15, miR-16, miR-17, miR-18, miR-19, miR-20, miR-21, miR-22, miR-23, miR-24, miR-25, miR-26, miR-27, miR-28, miR-29, miR-30, miR-31, miR-32, miR-33, miR-34, miR-92, miR-93, miR-94, miR-95, miR-96, miR-97, miR-98, miR-99, miR-100, miR101, miR-103, miR-104, miR-105, miR-106, miR-107, miR-109, miR-110, miR-111, miR-112, miR-113, miR-114, miR-116, miR-119, miR-122, miR-125, miR-126, miR-127, miR-129, miR-130, miR-132, miR-133, miR-134, miR-136, miR-138, miR-140, miR-141, miR-144, miR-145, miR-146, miR-147, miR-148, miR-149, miR-150, miR-151, miR-153, miR-154, miR-157, miR-158, miR-160, miR-162, miR-164, miR-172, miR-173, miR-174, miR-175, miR-176, miR-177, miR-178, miR-179, miR-180, miR-182, miR-183, miR-184, miR-185, miR-186, miR-187, miR-188, miR-189, miR-191, miR-192, miR-193, miR-195, miR-196, miR-197, miR-199, miR-201, miR-203, miR-205, and miR-224, or a precursor thereof.

6. The method of claim 4, wherein the mRNA comprises a sequence selected from the group consisting of SEQ ID Nos: 1, 3, 12, and 14-120.

7. The method of claim 1, wherein the target gene is selected from the group consisting of: dbl proto-oncogene; transforming growth factor beta 1; transforming growth factor alpha; v-myb myeloblastosis viral oncogene homolog; c-cbl proto-oncogene; snoI; activin beta E subunit; myogenic factor 5; fibroblast growth factor 9; RON encoding a tyrosine kinase; E3 ubiquitin ligase SMURF1; jagged 2; jun-B encoding the JUN-B protein; methyl-CpG binding domain protein 4; ZIP kinase; endomucin; ICE-protease activating factor; hairy and enhancer of split 1; transforming growth factor beta 3; enaptin mRNA; AMP deaminase; interleukin 1 alpha; E2F transcription factor 6; laminin alpha; polymerase (DNA-directed) alpha; leukocyte tyrosine kinase; homeo box D1; laminin gamma; tumor necrosis factor receptor superfamily member 1A; villin 2; frizzled homolog 5; ATP-dependent chromatin remodelling protein; MSX2 mRNA for transcription factor; adipose differentiation-related protein; myogenic factor 4; SRY (Sex determining Region Y)-box 5; Notch homolog 1; Human tyrosine kinase-type receptor; polymerase (DNA directed) theta; cAMP responsive element binding protein 3; timeless homolog; RAD52 homolog; toll-like receptor 4; SRY (Sex determining Region Y)-box 9; homeo box A5; cell division cycle 42 GTP binding protein; desmuslin; TFIIIC Box B-binding subunit; profilin 2; c-fms proto-oncogene; delta-like 1; fatty-acid-Coenzyme A ligase long-chain 5; discs large homolog-associated protein 2; TFIIH gene for transcription factor II H; RNA polymerase III subunit RPC; RecQ protein-like 5; METH2 protein; MOST2 protein; SRY (Sex determining Region Y)-box 7; integrin beta 1 subunit; desmin; protection of telomeres 1; H2.0-like homeo box 1; GABA transport protein; v-myc myelocytomatosis viral related oncogene neuroblastoma derived; BAG-family molecular chaperone regulator-S; Human placental bone morphogenic protein; retinoblastoma-associated factor 600; ALK-4; tolloid-like 2; RIGB; Human DNA repair helicase; T-box 22; BRCA1 associated protein 1; Sp3 transcription factor; TEF-1 gene; forkhead box A3; ets family transcription factor ELF2A; microtubule-associated protein 1A; myosin 5B; NEDD4-like ubiquitin ligase 1; Mint1 mRNA; PARX protein; epidermal growth factor receptor; matrix metalloproteinase 3; VE-cadherin; microtubule-associated protein 2; TAF7 RNA polymerase II TATA box binding protein (TBP)-associated factor; mitochondrial elongation factor G2; eyes absent homolog; paired box gene 3; synaptotagmin 1; histone deacetylase 5; homolog of Drosophila headcase; homeo box B8; fyn-related kinase; TGF-beta/activin signal transducer FAST-1 p; La autoantigen; mutL homolog 1; E74-like factor 3; B-myb gene; a-myb mRNA; jagged 1; homeobox protein SHOTb; death-associated protein kinase 3; RAD51 homolog (RecA homolog); methyl-CpG binding endonuclease; HUS1 checkpoint homolog; HES 1 protein; caldesmon 1; VENT-like homeobox 2; early growth response 2 protein; Notch3; lin-28 homolog; PML-3; c-myc binding protein; transducer of ERBB2 1; neuron navigator 3; multiple asters 1; headcase homolog; microtubule-associated protein 6; methyl-CpG binding domain protein 1; EphA5; polymerase (RNA) III (DNA directed); neuro-oncological ventral antigen 1; activating transcription factor 1; interphotoreceptor retinoid-binding protein; E2F transcription factor 3; mesoderm specific transcript homolog; bone morphogenetic protein 3; EphA3; methyl-CpG binding domain protein 5; fibroblast growth factor 12; RNA helicase A; matrix metalloproteinase 26; crossveinless-2; cadherin 5 type 2 VE-cadherin; eukaryotic translation initiation factor 4A; TWEAK; fork head domain protein; HOXB7 gene; Pax-3; homeobox protein SHOTa; inhibitor of growth family member 1; v-ets erythroblastosis virus E26 oncogene like; reticulon 4; NOD2 protein; interleukin 6 receptor; PML-2 mRNA; discs large homolog 1; Yes-associated protein 1; CD 14 antigen; negative differentiation regulator; CREB binding protein; v-ski sarcoma viral oncogene homolog; sidekick homolog 1; bone morphogenetic protein receptor type II; programmed cell death 10; cyclin H; nuclear protein double minute 1; BCL2/adenovirus E1B 19 kDa interacting protein 2; karyopherin beta 2; and v-ros UR2 sarcoma virus oncogene homolog 1.

8. The method of claim 7, wherein the mRNA transcribed from said target gene comprises a polynucleotide sequence having at least 70% identity to a polynucleotide selected from SEQ ID Nos: Nos: 5-11, 13, and 121-290.

9. The method of claim 7, wherein said target gene comprises a polynucleotide sequence that hybridizes under moderately stringent conditions with a polynucleotide sequence selected from SEQ ID Nos: 291-454.

10. A method for modulating expression of a mammalian target gene in a cell, the method comprising introducing into the cell an siRNA that forms a duplex region with an mRNA, or precursor thereof, wherein an mRNA transcribed from said target gene comprises an mRNA target region.

11. The method of claim 10, wherein the siRNA forms a duplex region with an mRNA, thereby inhibiting the mRNA from forming a second duplex region with mRNA transcribed from said target gene.

12. The method of claim 10, wherein the siRNA forms a duplex region with an mRNA precursor, thereby inhibiting the mRNA precursor from converting to mRNA.

13. The method of claim 10, wherein the mRNA target region comprises a sequence having at least about 70% identity to a polynucleotide selected from SEQ ID Nos: 5-11, 13, and 121-290.

14. The method of claim 10, wherein the mRNA or precursor thereof comprises a sequence having at least about 70% identity to a polynucleotide selected from SEQ ID Nos: 1, 3, 12, and 14-120.

15. The method of claim 14, wherein the mRNA or precursor thereof is selected from the group consisting of: miR-1, miR-2-1, miR-5, miR-7, miR-8, miR-11, miR-12, miR-13, miR-14, miR-15, miR-16, miR-17, miR-18, miR-19, miR-20, miR-21, miR-22, miR-23, miR-24, miR-25, miR-26, miR-27, miR-28, miR-29, miR-30, miR-31, miR-32, miR-33, miR-34, miR-92, miR-93, miR-94, miR-95, miR-96, miR-97, miR-98, miR-99, miR-100, miR101, miR-103, miR-104, miR-105, miR-106, miR-107, miR-109, miR-110, miR-111, miR-112, miR-113, miR-114, miR-116, miR-119, miR-122, miR-125, miR-126, miR-127, miR-129, miR-130, miR-132, miR-133, miR-134, miR-136, miR-138, miR-140, miR-141, miR-144, miR-145, miR-146, miR-147, miR-148, miR-149, miR-150, miR-151, miR-153, miR-154, miR-157, miR-158, miR-160, miR-162, miR-164, miR-172, miR-173, miR-174, miR-175, miR-176, miR-177, miR-178, miR-179, miR-180, miR-182, miR-183, miR-184, miR-185, miR-186, miR-187, miR-188, miR-189, miR-191, miR-192, miR-193, miR-195, miR-196, miR-197, miR-199, miR-201, miR-203, miR-205, and miR-224, or a precursor thereof.

16. The method of claim 14, wherein the mRNA or precursor thereof comprises a sequence selected from the group consisting of SEQ ID Nos: 1, 3, 12, and 14-120.

17. The method of claim 10, wherein the target gene is selected from the group consisting of: dbl proto-oncogene; transforming growth factor beta 1; transforming growth factor alpha; v-myb myeloblastosis viral oncogene homolog; c-cbl proto-oncogene; snoI; activin beta E subunit; myogenic factor 5; fibroblast growth factor 9; RON encoding a tyrosine kinase; E3 ubiquitin ligase SMURF1; jagged 2; jun-B encoding the JUN-B protein; methyl-CpG binding domain protein 4; ZIP kinase; endomucin; ICE-protease activating factor; hairy and enhancer of split 1; transforming growth factor beta 3; enaptin mRNA; AMP deaminase; interleukin 1 alpha; E2F transcription factor 6; laminin alpha; polymerase (DNA-directed) alpha; leukocyte tyrosine kinase; homeo box D1; laminin gamma; tumor necrosis factor receptor superfamily member 1A; villin 2; frizzled homolog 5; ATP-dependent chromatin remodelling protein; MSX2 mRNA for transcription factor; adipose differentiation-related protein; myogenic factor 4; SRY (Sex determining Region Y)-box 5; Notch homolog 1; Human tyrosine kinase-type receptor; polymerase (DNA directed) theta; cAMP responsive element binding protein 3; timeless homolog; RAD52 homolog; toll-like receptor 4; SRY (Sex determining Region Y)-box 9; homeo box A5; cell division cycle 42 GTP binding protein; desmuslin; TFIIIC Box B-binding subunit; profilin 2; c-fms proto-oncogene; delta-like 1; fatty-acid-Coenzyme A ligase long-chain 5; discs large homolog-associated protein 2; TFIIH gene for transcription factor II H; RNA polymerase III subunit RPC; RecQ protein-like 5; METH2 protein; MOST2 protein; SRY (Sex determining Region Y)-box 7; integrin beta 1 subunit; desmin; protection of telomeres 1; H2.0-like homeo box 1; GABA transport protein; v-myc myelocytomatosis viral related oncogene neuroblastoma derived; BAG-family molecular chaperone regulator-S; Human placental bone morphogenic protein; retinoblastoma-associated factor 600; ALK-4; tolloid-like 2; RIGB; Human DNA repair helicase; T-box 22; BRCA1 associated protein 1; Sp3 transcription factor; TEF-1 gene; forkhead box A3; ets family transcription factor ELF2A; microtubule-associated protein 1A; myosin 5B; NEDD4-like ubiquitin ligase 1; Mint1 mRNA; PARX protein; epidermal growth factor receptor; matrix metalloproteinase 3; VE-cadherin; microtubule-associated protein 2; TAF7 RNA polymerase II TATA box binding protein (TBP)-associated factor; mitochondrial elongation factor G2; eyes absent homolog; paired box gene 3; synaptotagmin 1; histone deacetylase 5; homolog of Drosophila headcase; homeo box B8; fyn-related kinase; TGF-beta/activin signal transducer FAST-1p; La autoantigen; mutL homolog 1; E74-like factor 3; B-myb gene; a-myb mRNA; jagged 1; homeobox protein SHOTb; death-associated protein kinase 3; RAD51 homolog (RecA homolog); methyl-CpG binding endonuclease; HUS1 checkpoint homolog; HES1 protein; caldesmon 1; VENT-like homeobox 2; early growth response 2 protein; Notch3; lin-28 homolog; PML-3; c-myc binding protein; transducer of ERBB2 1; neuron navigator 3; multiple asters 1; headcase homolog; microtubule-associated protein 6; methyl-CpG binding domain protein 1; EphA5; polymerase (RNA) III (DNA directed); neuro-oncological ventral antigen 1; activating transcription factor 1; interphotoreceptor retinoid-binding protein; E2F transcription factor 3; mesoderm specific transcript homolog; bone morphogenetic protein 3; EphA3; methyl-CpG binding domain protein 5; fibroblast growth factor 12; RNA helicase A; matrix metalloproteinase 26; crossveinless-2; cadherin 5 type 2 VE-cadherin; eukaryotic translation initiation factor 4A; TWEAK; fork head domain protein; HOXB7 gene; Pax-3; homeobox protein SHOTa; inhibitor of growth family member 1; v-ets erythroblastosis virus E26 oncogene like; reticulon 4; NOD2 protein; interleukin 6 receptor; PML-2 mRNA; discs large homolog 1; Yes-associated protein 1; CD14 antigen; negative differentiation regulator; CREB binding protein; v-ski sarcoma viral oncogene homolog; sidekick homolog 1; bone morphogenetic protein receptor type II; programmed cell death 10; cyclin H; nuclear protein double minute 1; BCL2/adenovirus E1B 19 kDa interacting protein 2; karyopherin beta 2; and v-ros UR2 sarcoma virus oncogene homolog 1.

18. The method of claim 17, wherein the mRNA transcribed from said target gene comprises a polynucleotide sequence having at least about 70% identity to a polynucleotide selected from SEQ ID Nos: 1, 3, 12, and 14-120.

19. The method of claim 17, wherein said target gene comprises a polynucleotide sequence that hybridizes under moderately stringent conditions with a polynucleotide sequence selected from SEQ ID Nos: 291-454.

20. The method of claim 1, further comprising measuring expression of said target gene.

21. The method of claim 1, wherein the method modulates ontogenesis, function, differentiation and/or viability of a mammalian cell.

22. A method for controlling ontogenesis of mammal, function of mammalian cell, differentiation of mammalian cell or viability of mammalian cell in the post-transcriptional phase, the method comprising introducing into the cell a mRNA or a siRNA silencing precursor to the the mRNA.

23. The method defined in claim 10, wherein the siRNA binds to a loop in stem-loop structure of the mRNA or precursor thereof.

24. The method of claim 10, wherein the siRNA targets mRNA and has a sequence with at least about 70% identity to the sequence disclosed in SEQ ID No: 2.

25. The method of claim 10, wherein the method controls differentiation of nerve cell by regulating expression of hairy and enhancer of split 1.

26. A plasmid vector comprising a promoter and a polynucleotide sequence expressing mRNA or a precursor to the miRNA.

27. A plasmid vector comprising from a promoter and a nucleotide sequence expressing siRNA silencing precursor to miRNA.

28. The plasmid vector of claim 26, wherein the miRNA is capable of forming a duplex region with an mRNA transcribed from a mammalian target gene.

29. The plasmid vector defined in claim 27, wherein the promoter is tRNA^{(val)} promoter.

30. The plasmid vector defined in claim 27, wherein the promoter is selected from the group consisting of tRNA^{(val)} promoter, U6 promoter, H1 promoter and Pol II promoter, such as CMV and SV40 promoter.

31. A method for controlling ontogenesis of mammal, function of mammalian cell, differentiation of mammalian cell or viability of mammalian cell in the post-transcriptional phase introducing into the cell the plasmid vector defined in claim 27.

32. A method for treating cancer, immune disease, nerve disorder or inflammatory disease, the method comprising introducing into a cell an miRNA, a siRNA silencing precursor to the mRNA or the plasmid vector defined in claim 27.

33. The method defined in claim 32, wherein the nerve disorder is selected from amyotrophic lateral sclerosis (ALS), Parkinson disease or Alzheimer disease.

34. A method for screening pharmaceuticals using a miRNA, a siRNA silencing precursor to the miRNA or the plasmid vector defined defined in claim 27.

35. The method defined in claim 34, wherein the target mRNA is derived from a recombinant gene having a sequence of the target region of the miRNA.

36. A method for gene function analysis using a miRNA, a siRNA silencing precursor to the miRNA or the plasmid vector defined in claim 27.

37. A method for regulation of cell differentiation to muscle cell, bone cell or myocardial cell, identified by the gene function analysis defined in claim 36.

38. A method for preservation or maintenance of anaplastic cell, introducing into cell a substance suppressing expression of miR-23.

39. A method for regulating ratio of gene expression, by producing recombinant of selected gene and target sequence of miR-23 of Hes1, and designing miR-23 sequence 50 to 90% complementary to the target sequence.

40. A method for suppressing gene expression, introducing into cell a siRNA inducing decomposition of mRNA and a miRNA.

41. The method defined in claim 40, wherein the miRNA is miR-23.
